(19) United States
(12) Patent Application Publication
(10) Pub. No.: US 2026/0108596 A1
(43) Pub. Date: Apr. 23, 2026

(54) NOBLE COLD-ADAPTED ATTENUATED MERS-COV

(71) Applicant: PIONEERVACCINE INC., Daejeon (KR)

(72) Inventors: Heejeong SEO, Daejeon (KR); Yunyueng JANG, Daejeon (KR)

(73) Assignee: PIONEERVACCINE INC., Daejeon (KR)

(21) Appl. No.: 18/571,645

(22) PCT Filed: Jun. 22, 2023
(86) PCT No.: PCT/KR2023/008704

§ 371 Date: Dec. 18, 2023

(30) Foreign Application Priority Data
Feb. 8, 2023 (KR) 10-2023-0016667

(51) Int. Cl. A61K 39/215; A61K 9/00; A61K 39/00; A61P 31/14; C07K 14/005; C12N 7/00

(52) U.S. Cl. CPC A61K 39/215; A61K 9/0043; A61P 31/14; C07K 14/005; C12N 7/00; A61K 2039/5254; A61K 2039/543; C12N 2770/20021; C12N 2770/20022; C12N 2770/20052

(57) ABSTRACT

The present invention relates to a novel cold-adapted attenuated Middle East respiratory syndrome coronavirus (MERS-CoV) and uses thereof. According to the present invention, the novel attenuated Middle East respiratory syndrome coronavirus (MERS-CoV) capable of effectively preventing infection with MERS-CoV has been developed by cold-adapting the MERS-CoV gradually, and thus can be effectively used as vaccines and therapeutic agents capable of preventing and treating Middle East respiratory syndrome.

### CROSS REFERENCE TO RELATED APPLICATIONS

This application is a National Stage of Application No. PCT/KR2023/008704 filed Jun. 22, 2023, claiming priority based on Korean Patent Application No. 10-2023-0016667 filed Feb. 8, 2023, the entire disclosures of which are incorporated herein by reference.

### INCORPORATION BY REFERENCE OF SEQUENCE LISTING

The content of the electronically submitted sequence listing, file name: Q294150_sequence listing as filed.xml; size: 110,410 bytes; and date of creation: Dec. 4, 2013, filed herewith, is incorporated herein by reference in its entirety.

### TECHNICAL FIELD

The present invention relates to a novel cold-adapted attenuated Middle East respiratory syndrome coronavirus (MERS-CoV) and uses thereof.

### BACKGROUND ART

Coronaviruses are known to mainly cause pneumonia and enteritis in humans and animals, and also known to occasionally cause nervous system infections and hepatitis. The coronaviruses belong to the Coronaviridae family and are positive sense RNA viruses with a spherical outer membrane and a size of approximately 100 to 120 nm. Based on a genetic structure, the coronaviruses are subdivided into four genera of alphacoronavirus, betacoronavirus, gammacoronavirus, and deltacoronavirus. Among them, human coronaviruses that cause severe acute respiratory infections in humans are severe acute respiratory syndrome coronavirus (SARS-CoV), Middle East respiratory syndrome coronavirus (MERS-CoV), and severe acute respiratory syndrome coronavirus 2 (SARS-CoV-2; coronavirus disease 2019, COVID-19), which belong all to betacoronaviruses. Among them, MERS-CoV is a type of betacoronavirus among coronaviruses, and has a gene length which varies, but is approximately 30 kb and has 11 open reading frames (ORFs). The structural proteins of MERS-CoV include spike (S), envelope (E), matrix (M), and nucleocapsid (NP) proteins.

Middle East respiratory syndrome (MERS) is an acute respiratory disease caused by infection with MERS-CoV, a novel coronavirus (CoV) belonging to the betacoronavirus genus, and has been known to the world while confirming novel coronavirus infection from patients with acute respiratory syndrome with symptoms of renal failure first in the Health Protection Agency of the UK (HPA) at September 2012, and reporting the confirmed novel coronavirus infection to the World Health Organization (WHO). Thereafter, patients were continuously reported based on countries on the Arabian Peninsula, including Saudi Arabia, and the WHO announced (on May 13, 2013) that the disease was called Middle East respiratory syndrome (MERS). Since the first patient occurred, the MERS has spread to some European countries such as France, Italy, and Greece, Tunisia in Africa, Malaysia and the Philippines in Asia, and recently the United States and the Netherlands as well as countries in the Middle East, mainly Saudi Arabia, and then until May 16, 2014, a total of 621 patients and 188 deaths (mortality rate of approximately 30%) were reported in a total of 19 countries. Among these, the number of patients occurring in Saudi Arabia and the United Arab Emirates accounts for 93% (578 patients) of the total patients. The sex ratio of MERS patients was 7:1 of male:female, with more males, and the average age was 48.5 years. The symptoms may also be mainly accompanied by respiratory symptoms such as fever, cough, and difficulty breathing, and acute renal failure, lymphopenia, and thrombocytopenia, and are known that the prognosis is poor for people with underlying diseases such as diabetes or cancer, people with reduced immune function, or the elderly. For the MERS, preventive vaccines and therapeutic agents (antivirals) have not yet been developed, and thus, symptomatic treatment was administered depending on the symptoms.

### DISCLOSURE

### Technical Problem

An object of the present invention is to provide a cold-adapted attenuated Middle East respiratory syndrome coronavirus (MERS-CoV).

Another object of the present invention is to provide a vaccine composition for preventing infection with MERS-CoV.

Yet another object of the present invention is to provide a pharmaceutical composition for preventing or treating Middle East respiratory syndrome (MERS).

Yet another object of the present invention is to provide a method for preventing infection with MERS-CoV.

Yet another object of the present invention is to provide a method for preventing or treating MERS.

Yet another object of the present invention is to provide a method for preparing a cold-adapted attenuated Middle East respiratory syndrome coronavirus (MERS-CoV).

Yet another object of the present invention is to provide a use of cold-adapted attenuated MERS-CoV for use in a vaccine composition.

Yet another object of the present invention is to provide a use of cold-adapted attenuated MERS-CoV for preventing or treating MERS.

### Technical Solution

An aspect of the present invention provides a novel cold-adapted attenuated Middle East respiratory syndrome coronavirus (MERS-CoV).

Another aspect of the present invention provides a vaccine composition for preventing infection with MERS-CoV.

Yet another aspect of the present invention provides a pharmaceutical composition for preventing or treating Middle East respiratory syndrome (MERS).

Yet another aspect of the present invention provides a method for preventing infection with MERS-CoV.

Yet another aspect of the present invention provides a method for preventing or treating Middle East respiratory syndrome (MERS).

Yet another aspect of the present invention provides a method for preparing a cold-adapted attenuated MERS-CoV.

Yet another aspect of the present invention provides a use of cold-adapted attenuated MERS-CoV for use in a vaccine composition.

Yet another aspect of the present invention provides a use of cold-adapted attenuated MERS-CoV for preventing or treating MERS.

### Advantageous Effects

According to the present invention, the novel attenuated Middle East respiratory syndrome coronavirus (MERS-CoV) capable of effectively preventing infection with MERS-CoV has been developed by cold-adapting the MERS-CoV gradually, and thus can be effectively used as vaccines and therapeutic agents capable of preventing and treating Middle East respiratory syndrome.

### DESCRIPTION OF DRAWINGS

FIGS. 1A to 1C are diagrams confirming the attenuation of a cold-adapted attenuated MERS-CoV live vaccine strain:
Wild-type MERS-CoV virus infected mice: Wild-type MERS-CoV strain EMC2012;
Cold-adapted MERS-CoV vaccine virus infected mice: EMC2012-CA22° C.;
PBS-mock uninfected mice: uninfected control group;

FIG. 1A: Survival rates of mice after infection;

FIG. 1B: Weight changes of mice after infection; and

FIG. 1C: Tissue-specific virus titers of mice after infection.

FIGS. 2A to 2I are diagrams of histopathological analysis performed on mice infected with a cold-adapted attenuated MERS-CoV live vaccine strain:

FIG. 2A: Brain tissue of PBS-mock mouse;

FIG. 2B: Brain tissue of mouse infected with wild-type MERS-CoV (EMC2012) (arrow: lymphocyte infiltration);

FIG. 2C: Brain tissue of mouse infected with EMC2012-CA22° C.;

FIG. 2D: Kidney tissue of PBS-mock mouse;

FIG. 2E: Kidney tissue of mouse infected with wild-type MERS-CoV (EMC2012);

FIG. 2F: Kidney tissue of mouse infected with EMC2012-CA22° C.;

FIG. 2G: Lung tissue of PBS-mock mouse;

FIG. 2H: Lung tissue of mouse infected with wild-type MERS-CoV (EMC2012); and

FIG. 2I: Lung tissue of mouse infected with EMC2012-CA22° C.

FIGS. 3A to 3D are diagrams confirming the temperature sensitivity, mucosal IgA antibody induction, cellular immunity, and cytokine characteristics of a cold-adapted attenuated MERS-CoV live vaccine strain in mice:

FIG. 3A: Temperature sensitivity of EMC2012-CA22° C.;

FIG. 3B: MERS-CoV-specific IgA antibody titers in tissues of immunized mouse;

FIG. 3C: Number of IFN-γ-expressing lymphocytes in splenocytes of immunized mouse; and

FIG. 3D: Cytokines in splenocytes of immunized mouse.

FIGS. 4A to 4D are diagrams confirming neutralizing antibody induction and infection prevention effects in mice immunized with a cold-adapted attenuated MERS-CoV live vaccine strain:

FIG. 4A: Neutralizing antibody titers in mouse serum after immunization with live vaccine EMC2012-CA22° C.;

FIG. 4B: Survival rate of mouse after infection with wild-type MERS-CoV (Korean MERS-CoV/2015) after immunization with live vaccine EMC2012-CA22° C.;

FIG. 4C: Weight change of mouse after infection with wild-type MERS-CoV (Korean MERS-CoV/2015) after immunization with live vaccine EMC2012-CA22° C.; and

FIG. 4D: Virus titers in tissues of mouse after infection with wild-type MERS-CoV (Korean MERS-CoV/2015) after immunization with live vaccine EMC2012-CA22° C.

FIGS. 5A to 5I are diagrams of histopathological analysis performed on mice after infection with wild-type MERS-CoV (Korean MERS-CoV/2015) after immunization with a cold-adapted attenuated MERS-CoV live vaccine strain:

FIG. 5A: Brain tissue of PBS-mock mouse;

FIG. 5B: Brain tissue of mouse infected with wild-type MERS-CoV (Korean MERS-CoV/2015) after non-immunization (arrow: lymphocyte infiltration);

FIG. 5C: Brain tissue of mouse infected with wild-type MERS-CoV (Korean MERS-CoV/2015) after immunization with live vaccine EMC2012-CA22° C.;

FIG. 5D: Kidney tissue of PBS-mock mouse;

FIG. 5E: Kidney tissue of mouse infected with wild-type MERS-CoV (Korean MERS-CoV/2015) after non-immunization;

FIG. 5F: Kidney tissue of mouse infected with wild-type MERS-CoV (Korean MERS-CoV/2015) after immunization with live vaccine EMC2012-CA22° C.;

FIG. 5G: Lung tissue of PBS-mock mouse;

FIG. 5H: Lung tissue of mouse infected with wild-type MERS-CoV (Korean MERS-CoV/2015) after non-immunization; and

FIG. 5I: Lung tissue of mouse infected with wild-type MERS-CoV (Korean MERS-CoV/2015) after immunization with live vaccine EMC2012-CA22° C.

### BEST MODE OF THE INVENTION

Hereinafter, an embodiment of the present invention will be described in detail with reference to the accompanying drawings. However, the following embodiments are presented as examples for the present invention, and when it is determined that a detailed description of well-known technologies or configurations known to those skilled in the art may unnecessarily obscure the gist of the present invention, the detailed description thereof may be omitted, and the present invention is not limited thereto. Various modifications and applications of the present invention are possible within the description of claims to be described below and the equivalent scope interpreted therefrom.

Terminologies used herein are terminologies used to properly express embodiments of the present invention, which may vary according to a user, an operator's intention, customs in the art to which the present invention pertains, or the like. Therefore, the definitions of these terminologies used herein will be defined based on the contents throughout the specification. Throughout the specification, unless explicitly described to the contrary, when a certain part “comprises” a certain component, it will be understood to imply the inclusion of stated elements but not the exclusion of any other elements.

All technical terms used in the present invention, unless otherwise defined, have the meaning as commonly understood by those skilled in the related art of the present invention. In addition, although preferred methods and samples are described herein, similar or equivalent methods and samples thereto are also included in the scope of the present invention. The contents of all publications disclosed as references in this specification are incorporated in the present invention.

Throughout the present specification, general one-letter or three-letter codes for naturally existing amino acids are used, and generally allowed three-letter codes for other amino acids, such as α-aminoisobutyric acid (Aib) and N-methylglycine (Sar) are also used. The amino acids mentioned herein as abbreviations are also described as follows according to the IUPAC-IUB nomenclature.

Alanine: A, Arginine: R, Asparagine: N, Aspartic acid: D, Cysteine: C, Glutamic acid: E, Glutamine: Q, Glycine: G, Histidine: H, Isoleucine: I, Leucine: L, Lysine: K, Methionine: M, Phenylalanine: F, Proline: P, Serine: S, Threonine: T, Tryptophan: W, Tyrosine: Y, and Valine: V.

In an aspect, the present invention relates to a cold-adapted attenuated MERS-CoV including a gene encoding an amino acid in which at least amino acid selected from the group consisting of amino acids at positions 79, 1616, 2088, 2210, 3295, 3735, 3822, 4195, and 4247 in ORF1a polyprotein; positions 38, 66, 305, 872, 879, and 1251 in S protein; and position 5 in M protein of wild type Middle East respiratory syndrome coronavirus (MERS-CoV) is substituted with a sequence different from a wild-type amino acid.

In an embodiment, the cold-adapted attenuated MERS-CoV of the present invention may include a gene encoding an amino acid including at least one amino acid substitution selected from the group consisting of H79R, H1616L, T2088P, A2210V, Q3295R, F3735I, E3822G, P4195S, and N4247S in the ORF1a polyprotein; T38P, N66Y, S305R, T872A, I879T, and S1251F in the S protein; and T5M in the M protein.

In an embodiment, in the wild-type MERS-CoV, the ORF1a polyprotein may include an amino acid sequence represented by SEQ ID NO: 3 or 4, the S protein may include an amino acid sequence represented by SEQ ID NO: 5, and the M protein may include an amino acid sequence represented by SEQ ID NO: 11.

In an embodiment, the wild-type MERS-CoV may include NS3 protein including an amino acid sequence represented by SEQ ID NO: 6, NS4A protein including an amino acid sequence represented by SEQ ID NO: 7, NS4B protein including an amino acid sequence represented by SEQ ID NO: 8, NS5 protein including an amino acid sequence represented by SEQ ID NO: 9, E protein (envelope protein) including an amino acid sequence represented by SEQ ID NO: 10, nucleocapsid protein including an amino acid sequence represented by SEQ ID NO: 12, and ORF8b protein including an amino acid sequence represented by SEQ ID NO: 13 (Table 4).

In an embodiment, the cold-adapted attenuated MERS-CoV of the present invention may include a gene encoding ORF1a polyprotein including an amino acid sequence represented by SEQ ID NO: 14 or 15, a gene encoding S protein including an amino acid sequence represented by SEQ ID NO: 16, and a gene encoding M protein including an amino acid sequence represented by SEQ ID NO: 22.

In an embodiment, the cold-adapted attenuated MERS-CoV of the present invention may include NS3 protein including an amino acid sequence represented by SEQ ID NO: 17, NS4A protein including an amino acid sequence represented by SEQ ID NO: 18, NS4B protein including an amino acid sequence represented by SEQ ID NO: 19, NS5 protein including an amino acid sequence represented by SEQ ID NO: 20, E protein (envelope protein) including an amino acid sequence represented by SEQ ID NO: 21, nucleocapsid protein including an amino acid sequence represented by SEQ ID NO: 23, and ORF8b protein including an amino acid sequence represented by SEQ ID NO: 24 (Table 4).

In an embodiment, the wild-type MERS-CoV may include a nucleotide sequence represented by SEQ ID NO: 2.

In an embodiment, the cold-adapted attenuated MERS-CoV of the present invention may include a nucleotide sequence represented by SEQ ID NO: 1.

An amino acid mutation position of the present invention starts from position 1 of the amino acid sequence of each reference protein. For example, in the amino acid sequence represented by SEQ ID NO: 11 which is the sequence of the M protein of the wild-type MERS-CoV of the present invention, threonine (T, Thr) as the fifth amino acid is the same as T5, and in the amino acid sequence represented by SEQ ID NO: 22 which is the sequence of the M protein of the cold-adapted attenuated MERS-CoV of the present invention, methionine (M, Met) as the fifth amino acid is the same as 5M.

As used herein, the term “amino acid modification/mutation” refers to substitution, insertion and/or deletion, preferably substitution of amino acids in a polypeptide sequence. As used herein, the term “amino acid substitution” or “substitution” means that an amino acid at a specific position in the polypeptide sequence from which a gene of the corresponding virus has been translated is replaced with another amino acid. For example, T5M substitution means that the 5th amino acid residue, threonine, is replaced with methionine in the amino acid sequence represented by SEQ ID NO: 4, in which the nucleotide sequence (SEQ ID NO: 3) of wild-type MERS-CoV is translated.

In an embodiment, the cold-adapted attenuated MERS-CoV of the present invention may be prepared by infecting cells with wild-type MERS-CoV and then adapting the cells gradually from 37° C. to 22° C., and the cells may be Vero cells, Calu-3, A549, HUH7.0 or HEK-293T cells.

In an aspect, the present invention provides a vaccine composition for preventing infection with Middle East respiratory syndrome coronavirus (MERS-CoV), including the cold-adapted attenuated MERS-CoV of the present invention.

In an embodiment, a host animal in which the vaccine composition of the present invention may cause an immune response may be mammal or bird, for example, may include human, dog, cat, pig, horse, chicken, duck, turkey, ferret, etc.

In an embodiment, the vaccine composition of the present invention may be a live vaccine, or may be a live attenuated vaccine.

As used herein, the term “live vaccine” refers to a vaccine containing live viral active ingredients. In addition, the term “attenuation” refers to artificially weakening the toxicity of a living pathogen, and means that a gene involved in the essential metabolism of the pathogen is mutated so that a disease in the body is not caused and only an immune system is stimulated to induce immunity. The attenuation of the virus may be achieved by ultraviolet (UV) irradiation, chemical treatment, or high-order serial subculture in vitro. The attenuation may also be achieved by making distinct genetic changes, for example, by specific deletion of a viral sequence known to provide toxicity or insertion and mutation of a sequence into a viral genome.

In an embodiment, the vaccine composition of the present invention may be administered intranasally.

The vaccine composition of the present invention may be prepared as an oral or parenteral formulation and may be administered, for example, by intradermal, intramuscular, intraperitoneal, intranasal or epidural routes, preferably intranasal route, but is not limited thereto.

In an embodiment, the vaccine composition may further include an adjuvant (immune booster).

The adjuvant refers to a compound or mixture that enhances the immune response and/or accelerates the absorption rate after inoculation and includes an optional absorption accelerator. Acceptable adjuvants include Freund's complete adjuvant, Freund's incomplete adjuvant, saponin, mineral gel such as aluminum hydroxide, surfactant such as lysolecithin, pluronic polyol, polyanion, peptide, oil or hydrocarbon emulsion, Keyhole Limpet hemocyanin, dinitrophenol, etc., but are not limited thereto.

The vaccine composition of the present invention may further include one or more selected from the group consisting of solvents, adjuvants, and excipients. The solvent includes physiological saline or distilled water, the adjuvant includes Freund's incomplete or complete adjuvant, aluminum hydroxide gel, vegetable and mineral oils, etc., and the excipient includes aluminum phosphate, aluminum hydroxide, or aluminum potassium sulfate, but is not limited thereto, and may further include substances used in vaccine preparation that are well known to those skilled in the art. Suitable carriers for the vaccine are known to those skilled in the art and include proteins, sugars, etc., but are not limited thereto. The carrier may be an aqueous solution or a non-aqueous solution, a suspension or emulsion. As an adjuvant to increase immunogenicity, morphous or amorphous organic or inorganic polymers, etc. may be used. The adjuvants are generally known to playa role in promoting immune responses through chemical and physical binding to antigens. The immune composition may be used as a composition for inducing optimal immune responses by combining various adjuvants and additives for promoting immune responses. In addition, the composition to be added to the vaccine may use stabilizers, inactivators, antibiotics, preservatives, etc. Depending on the administration route of the vaccine, the vaccine antigens may be mixed and used with distilled water, buffer solutions, etc.

As used herein, the term “prevention” refers to all actions that inhibit or delay the occurrence, spread, and recurrence of the infection with MERS-CoV by administration of the composition according to the present invention.

The vaccine composition of the present invention may be included in an appropriate amount depending on the weight, age, severity of symptoms, etc. of a subject to be administered, for example, 2×10³ to 2×10⁵ pfu, but is not limited thereto.

As used herein, the term “vaccine” refers to a pharmaceutical composition containing at least one immunologically active ingredient that induces immunological responses in animals. The immunologically active ingredient of the vaccine may contain appropriate elements of live or dead cell lines (subunit vaccine). Thus, these elements are prepared by a purification step of lysing the entire cell line or growth culture thereof and then obtaining desired construct(s), or not limited thereto, but by a synthetic process induced by appropriate manipulation of an appropriate system, such as viruses, bacteria, insects, mammals or other species, followed by isolation and purification, or induction of the synthetic process in animals in need of the vaccine by direct incorporation of a genetic material using an appropriate pharmaceutical composition (polynucleotide vaccination). The vaccine may include one or simultaneously one or more of the elements described above.

The vaccine may be in any form known in the art, for example, a liquid and injection form or a solid form suitable for a suspension, but is not limited thereto. These formulations may also be emulsified or encapsulated in liposomes or soluble glasses or also prepared in an aerosol or spray form. These formulations may also be contained in transdermal patches. If necessary, the liquid or injection may contain propylene glycol and a sufficient amount (e.g., about 1%) of sodium chloride to prevent hemolysis.

In an aspect, the present invention provides a pharmaceutical composition for preventing or treating Middle East respiratory syndrome (MERS) including the cold-adapted attenuated MERS-CoV of the present invention.

As used herein, the term “prevention” means all actions that inhibit or delay the MERS by administering the composition.

As used herein, the term “treatment” means all actions that improve or beneficially change the symptoms of MERS and complications thereof by administration of the composition according to the present invention. Those skilled in the art to which the present invention pertains may determine the degree of improvement, enhancement and treatment by finding the exact criteria of a disease for which the composition of the present invention is effective.

The composition for treatment of the present invention may be administered parenterally (e.g., applied intravenously, subcutaneously, intraperitoneally or topically) or orally according to a desired method, and the range of the dose may vary depending on the weight, age, sex, and health condition of a patient, a diet, an administration time, an administration method, an excretion rate, the severity of a disease, etc. A daily dose of the composition according to the present invention is 0.0001 to 10 mg/ml, preferably 0.0001 to 5 mg/ml, and more preferably administered once to several times a day.

As used herein, the term “therapeutically effective dose” used in combination with the active ingredients means an amount of a pharmaceutically acceptable salt of a therapeutic agent effective for preventing or treating a target disease, and the therapeutically effective dose of the composition of the present invention may vary depending on many factors, such as an administration method, a target site, a condition of a subject with a disease, and the like. Accordingly, when used in the subject, the dose should be determined as an appropriate amount in consideration of both safety and efficiency.

The composition of the present invention is administered in a pharmaceutically effective dose. As used herein, the term “pharmaceutically effective dose” refers to an amount enough to treat the disease at a reasonable benefit/risk ratio applicable to medical treatment and not to cause side effects. An effective dose level may be determined according to factors including the health condition of a subject, the type and severity of a disease, the activity of a drug, the sensitivity to a drug, a method of administration, a time of administration, a route of administration, an excretion rate, duration of treatment, and combined or simultaneously used drugs, and other factors well-known in the medical field. The therapeutic agent of the present invention may be administered as an individual therapeutic agent or in combination with other therapeutic agents, and may be administered sequentially or simultaneously with existing therapeutic agents, and may be administered singly or multiply. It is important to administer an amount capable of obtaining a maximum effect with a minimal amount without side effects by considering all the factors, which may be easily determined by those skilled in the art.

The composition of the present invention may further include a pharmaceutically acceptable additive. At this time, the pharmaceutically acceptable additive may use starch, gelatinized starch, microcrystalline cellulose, lactose, povidone, colloidal silicon dioxide, calcium hydrogen phosphate, lactose, mannitol, syrup, gum arabic, pregelatinized starch, corn starch, powdered cellulose, hydroxypropyl cellulose, Opadry, sodium starch glycolate, lead carnauba, synthetic aluminum silicate, stearic acid, magnesium stearate, aluminum stearate, calcium stearate, sucrose, dextrose, sorbitol, talc and the like. The pharmaceutically acceptable additive according to the present invention is preferably included in an amount of 0.1 part by weight to 90 parts by weight based on the composition, but is not limited thereto.

The composition of the present invention may also include a carrier, a diluent, an excipient, or a combination of two or more thereof, which are commonly used in biological agents. The pharmaceutically acceptable carrier is not particularly limited as long as the carrier is suitable for in vivo delivery of the composition, and may be used by combining, for example, compounds described in Merck Index, 13th ed., Merck & Co. Inc., saline, sterile water, a Ringer's solution, buffered saline, a dextrose solution, a maltodextrin solution, glycerol, ethanol, and one or more of these ingredients, and if necessary, other conventional additives such as an antioxidant, a buffer, and a bacteriostat may be added. In addition, the pharmaceutical composition may be prepared in injectable formulations such as an aqueous solution, a suspension, and an emulsion, pills, capsules, granules, or tablets by further adding a diluent, a dispersant, a surfactant, a binder, and a lubricant. Furthermore, the pharmaceutical composition may be prepared preferably according to each disease or ingredient using a suitable method in the art or a method disclosed in Remington's Pharmaceutical Science (Mack Publishing Company, Easton PA, 18th, 1990).

Liquid formulations for oral administration of the composition of the present invention correspond to suspensions, internal solutions, emulsions, syrups, etc., and may include various excipients, such as wetting agents, sweeteners, fragrances, preservatives, and the like in addition to water and liquid paraffin, which are commonly used simple diluents. Formulations for parenteral administration include sterilized aqueous solutions, non-aqueous solvents, suspensions, emulsions, lyophilized agents, suppositories, and the like.

In an aspect, the present invention relates to a method of preventing infection with Middle East respiratory syndrome coronavirus (MERS-CoV) by administering the vaccine composition of the present invention to a subject other than a human.

In an aspect, the present invention relates to a method of preventing or treating Middle East respiratory syndrome by administering the pharmaceutical composition of the present invention for preventing or treating Middle East respiratory syndrome to a subject other than a human.

In an aspect, the present invention relates to a method for preparing cold-adapted attenuated Middle East respiratory syndrome coronavirus (MERS-CoV) including infecting cells with MERS-CoV and then adapting the cells gradually from 37° C. to 22° C.; and subculturing the adapted MERS-CoV at 22° C. and then collecting the subcultured MERS-CoV.

In an embodiment, the adapting of the cells gradually from 37° C. to 22° C. may be adapting the virus by lowering the temperature to the next lower temperature when the infected cells show a cytopathic effect (CPE).

In an aspect, the present invention relates to a use of cold-adapted attenuated Middle East respiratory syndrome coronavirus (MERS-CoV) of the invention for use in a vaccine composition.

In an aspect, the present invention relates to a use for preventing or treating MERS of cold-adapted attenuated MERS-CoV of the present invention.

### MODES FOR THE INVENTION

Hereinafter, the present invention will be described in more detail with reference to the following Examples. However, the following Examples are only intended to embody the contents of the present invention, and the present invention is not limited thereto.

### Example 1. Development and Analysis of Cold-Adapted Attenuated MERS-CoV Live Vaccine Strain

### 1-1. Development of Cold-Adapted Attenuated MERS-CoV Live Vaccine Strain

In order to develop a live MERS-CoV vaccine strain that may be used as a live attenuated vaccine strain or an inactivated split vaccine strain, MERS-CoV (EMC2012) was adapted gradually from 37° C. to 22° C. using Vero cells in a BSL3 facility. Specifically, while Vero cells were infected with MERS-CoV and cultured in an MEM medium supplemented with 1.5% bovine serum albumin (BSA) (Rocky Mountain Biologicals, Missoula, MT, USA) and 1×antibiotic-antifungal solution (Sigma) under a condition of 5% CO₂, when the infected Vero cells showed a cytopathic effect (CPE), the virus was adapted gradually from 37° C. to 22° C. by adapting the virus to the next lower temperature. At this time, the CPE usually appeared in the infected cells in about 3 to 10 days. When the gradually cold-adapted MERS-CoV was successfully cultured at 22° C. for at least 5 passes (>passage 5), a plaque was purified, which was named a cold-adapted attenuated MERS-CoV vaccine strain EMC2012-CA22° C.

### 1-2. Confirmation of Genetic Change of Cold-Adapted Attenuated MERS-CoV Live Vaccine Strain

In order to confirm the genetic characteristics of the cold-adapted attenuated MERS-CoV vaccine strain EMC2012-CA22° C. developed in Example 1-1, genomes of five purified clones were sequenced to determine a genetic difference from a virus MERS-CoV (EMC2012) before cold-adaptation. Specifically, a MERS-CoV (EMC2012-CA22° C.) stock (200 μl) cultured in Vero cells using a MEM medium was lysed with 350 μl of Buffer RLT, and then added and mixed with 500 μl of 70% ethanol. 700 μl of the reactant was transferred to an RNeasy Mini spin column, and the column was centrifuged at 13,500 rpm for 15 seconds in a microcentrifuge. After discarding a flow through, 700 μl of an RW1 buffer was added to the spin column and centrifuged at 13,500 rpm for 15 seconds. After discarding the flow through, 500 μl of an RPE buffer was added to the spin column and centrifuged at 13,500 rpm for 2 minutes. The spin column was transferred to a new 1.5 mL tube, and viral genomic RNA was eluted with RNAse-free water (50 μl). The eluted RNA was converted using the GoScript Reverse Transcription System (Promega, Madison, USA) and 12 reverse primers (Table 2), and then PCR-amplified with a segment-specific primer set (Table 3). PCR products were electrophoresed on an agarose gel and extracted with a QIAquick Gel Extraction Kit (QIAGEN). The extracted genes were cloned with a pGEM-T Easy vector (Promega) and transformed into E. coli DH5a (Enzynomics, Daejeon, Korea). Five successfully cloned plasmids were extracted and sequenced using a HiGene Plasmid Mini Prep Kit (BIOFACT, Daejeon, Korea). The sequenced genes were analyzed by DNASTAR Lasergene (Madison, Wisconsin, USA), and the analyzed whole nucleotide sequence of MERS-CoV (EMC2012-CA22° C.) was represented by SEQ ID NO: 1. In addition, the whole genome sequence was translated and compared with a gene (SEQ ID NO: 2) of wild type MERS-CoV (EMC2012) (GenBank accession number: NC_019843.3).

TABLE 1

| | | | Number of |
|---|---|---|---|
| Protein name | | Mutated amino acid sequence | mutated sequences |
| ORF1a | nsp1 | H79R | 1/193 |
| polyprotein | nsp3 | H1616L, T2088P, A2210V | 3/1887 |
| | msp5 | Q3295R | 1/306 |
| | nsp6 | F3735I, E3822G | 2/292 |
| | nsp9 | P4195S | 1/110 |
| | nsp10 | N4247S | 1/140 |

| | | |
|---|---|---|
| S protein | T38P, N66Y, S305R, T872A, | 6/1354 |
| | I879T, S1251F | |
| M protein | T5M | 1/220 |

Total amino acids 16/12,987 (including non-mutated ORFs)

TABLE 2

| Primer | Sequence (5′->3′) |
|---|---|
| 2500R | gggaatattagagactccctgccg |
| 5000R | ccacccatggactgcagccttaag |
| 7500R | ttgctgtgatataaaacgtacgtt |
| 10,000R | atgctacagttgggtggttggtaa |
| 12,500R | gggatatgtgactacctgattcca |
| 15,000R | atggcaaaaagttcatcttgctcc |
| 17,500R | gttacacatcaatctagtgacact |
| 20,000R | tgatttttctatcagaaataaaga |
| 22,500R | agtggagttgtgacaaatcattaa |
| 25,000R | tcaacaatcctagtgttattagtt |
| 27,500R | agctcggggcgattatgtgaagag |
| 30,119R | ttttttttttttgcaaatcatctaattagcct |

TABLE 3

| Seg- | Forward | Forward | Reverse | Reverse |
|---|---|---|---|---|
| ment | Primer | sequence | primer | sequence |
| 1 | 1F | gatttaag | 2500R | gggaatat |
| | | tgaatagc | | tagagact |
| | | ttggctat | | ccctgccg |
| 2 | 2400F | ttgcttaa | 5000R | ccacccat |
| | | taagggta | | ggactgca |
| | | tgcaactt | | gccttaag |
| 3 | 4900F | tgactgct | 7500R | ttgctgtg |
| | | gatgaaac | | atataaaa |
| | | aaaggcgc | | cgtacgtt |
| 4 | 7400F | agatacgg | 10,000R | atgctaca |
| | | catgcttg | | gttgggtg |
| | | ctctgcta | | gttggtaa |
| 5 | 9900F | gccgctta | 12,500R | gggatatg |
| | | tcgtgaag | | tgactacc |
| | | ctgcagca | | tgattcca |
| 6 | 12,400F | atggttgt | 15,000R | atggcaaa |
| | | atacctct | | aagttcat |
| | | tagtgtca | | cttgctcc |
| 7 | 14,900F | aatttaga | 17,500R | gttacaca |
| | | caagagtg | | tcaatcta |
| | | ctggccat | | gtgacact |
| 8 | 17,400F | atgtagga | 20,000R | tgattttt |
| | | gatccagc | | ctatcaga |
| | | acagttgc | | aataaaga |
| 9 | 19,900F | taattcag | 22,500R | agtggagt |
| | | ctttgaat | | tgtgacaa |
| | | atatgttt | | atcattaa |
| 10 | 22,400F | atataaac | 25,000R | tcaacaat |
| | | ttcaaccg | | cctagtgt |
| | | ttaacttt | | tattagtt |
| 11 | 24,900F | gttgtttc | 27,500R | agctcggg |
| | | tgcttatg | | gcgattat |
| | | gtctttgc | | gtgaagag |
| 12 | 27,400F | cctagttt | 30,119R | tttttttt |
| | | ctgtaact | | ttttgcaa |
| | | gacttctc | | atcatcta |
| | | | | attagcct |

TABLE 4

| | | | Wild-type | |
|---|---|---|---|---|
| | | | MERS-CoV | EMC2012- |
| protein | gene | location | (EMC2012) | CA22° C. |
| 1A polyprotein | orf1a | 279 . . . 13454 | SEQ ID NO: 3 | SEQ ID NO: 14 |
| 1AB polyprotein | orf1ab | join(279 . . . 13433, | SEQ ID NO: 4 | SEQ ID NO: 15 |
| | | 13433 . . . 21514) | | |
| spike protein (S protein) | S | 21456 . . . 25517 | SEQ ID NO: 5 | SEQ ID NO: 16 |
| NS3 protein | orf3 | 25532 . . . 25843 | SEQ ID NO: 6 | SEQ ID NO: 17 |
| NS4A protein | orf4a | 25852 . . . 26181 | SEQ ID NO: 7 | SEQ ID NO: 18 |
| NS4B protein | orf4b | 26093 . . . 26833 | SEQ ID NO: 8 | SEQ ID NO: 19 |
| NS5 protein | orf5 | 26840 . . . 27514 | SEQ ID NO: 9 | SEQ ID NO: 20 |
| envelope protein | E | 27590 . . . 27838 | SEQ ID NO: 10 | SEQ ID NO: 21 |
| membrane protein | M | 27853 . . . 28512 | SEQ ID NO: 11 | SEQ ID NO: 22 |
| nucleocapsid protein | N | 28566 . . . 29807 | SEQ ID NO: 12 | SEQ ID NO: 23 |
| ORF8b protein | orf8b | 28762 . . . 29100 | SEQ ID NO: 13 | SEQ ID NO: 24 |

As a result, it was confirmed that 16 amino acids of a total of 12,987 amino acids (AAs) translated from the gene of the MERS-CoV vaccine strain (EMC2012-CA22° C.) had amino acid substitution mutations in open reading frame (ORF) 1a protein, S protein, and M protein (Table 1). Specifically, nine amino acids were mutated in the OFR1a polyprotein, six amino acids were mutated in the S protein, and one amino acid was mutated in the M protein. Among six amino acid mutations T38P, N66Y, S305R, T872A, I879T, and S1251F in the S protein, there was no amino acid mutation at (367 to 606) in a receptor binding domain (RBD) of the MERS-CoV spike protein. In addition, there were confirmed mutation H79R in non-structural protein 1 (nsp1) involved in host immune response and suppression of host gene expression; mutations H1616L, T2088P and A2210V in nsp3, which were required for a replication and transcription complex of coronavirus; mutation Q3295R in nsp5 involved in cleavage of coronavirus polypeptide 1a/1ab to generate mature nsp4-6 of coronavirus; mutations F3735I and E3822G in nsp6 involved in antagonizing pI interferon responses in infected cells; mutation P4195S in nsp9, which acted as an essential component of the replication and transcription complex of coronavirus; and mutation N4247S in nsp10 which acted as viral exonuclease and reduced the mutation rate of error-prone RNA-dependent RNA polymerase of coronavirus. In addition, the T5M mutation was confirmed in the M protein, a structural component of MERS-CoV.

### 1-3. Measurement of Plaque Forming Unit (PFU)

The PFU of a MERS-CoV stock (EMC2012 or EMC2012-CA22° C.) was analyzed by plaque assay. Specifically, the viruses were serially diluted 10-fold in MEM supplemented with 1.5% BSA, then infected into Vero cells cultured in a 6-well plate in an incubator (5% CO₂, 35° C.) for 4 hours, and the infected Vero cells were overlaid with MEM (LPS Solution, Korea) containing 1% electrophoresis agar and incubated for 4 days. After incubation, the cells were stained with 0.10% crystal violet (Sigma-Aldrich, St. Louis, MO, USA) dissolved in a formaldehyde solution (37%), and then the number of plaques was counted.

### Example 2. Confirmation of Attenuation of EMC2012-CA22° C.

### 2-1. Confirmation of Pathogenic Attenuation

In order to determine whether the pathogenicity of the cold-adapted attenuated MERS-CoV vaccine strain EMC2012-CA22° C. has been attenuated, K18-hDPP4 mice (13 per group) were anesthetized with isoflurane USP (Gujarat, India) and then infected intranasally (i.n.) with 50 μl (2×10⁴ pfu) of the cold-adapted attenuated MERS-CoV vaccine strain EMC2012-CA22° C. or the wild-type MERS-CoV strain EMC2012, respectively. As a control group, PBS-mock-infected mice were used. The weight changes and mortality rates of the infected mice were observed for 14 days.

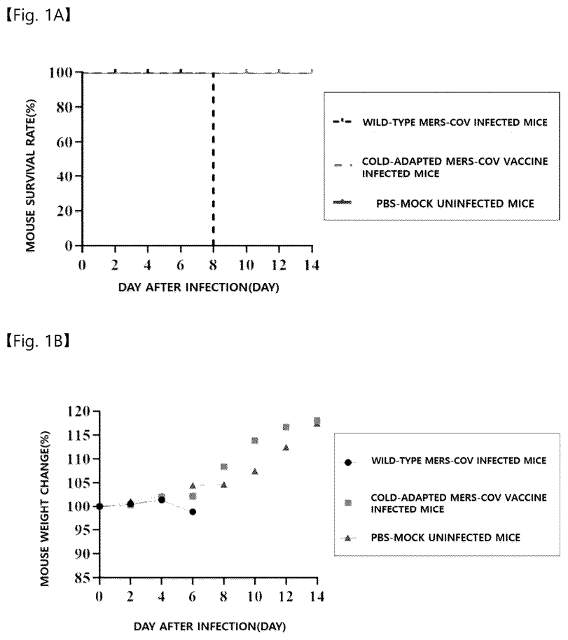

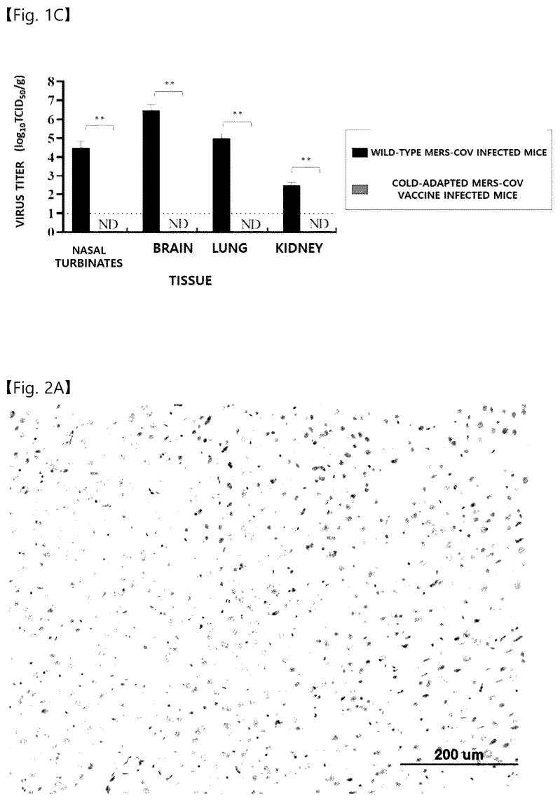

As a result, no death or weight loss occurred in a group of virus-uninfected PBS-mock mice and a group of K18-hDPP4 mice infected with the cold-adapted attenuated MERS-CoV vaccine strain EMC2012-CA22° C., whereas a group of K18-hDPP4 mice infected with the wild-type MERS-CoV strain EMC2012 100%-died by 8 days after infection and showed a maximum weight loss of 1.2% (FIGS. 1A and 1B).

### 2-2. Measurement of Virus Titers in Tissues

Six days after infection in Example 2-1 (p.i.), three animals per group were sacrificed and each tissue (nasal turbinates, brain, lung, and kidney) sample (0.1 g) was homogenized with a BeadBlaster homogeniser (Benchmark Scientific, Edison, New Jersey, USA) in 1 mL PBS (pH 7.4) and the virus titer was measured as log₁₀TCID₅₀/g.

As a result, high virus titers were detected in the tissues of the K18-hDPP4 mouse group (n=3) infected with the wild-type MERS-CoV strain EMC2012 (nasal turbinates: 4.5 TCID₅₀/g; brain: 6.5 TCID₅₀/g; lung: 5.0 TCID₅₀/g; and kidney: 2.5 TCID₅₀/g), but the virus was detected at a detection limit of less than 1 TCID₅₀/g in the tissues of the K18-hDPP4 mouse group (n=3) infected with the cold-adapted attenuated MERS-CoV vaccine strain EMC2012-CA22° C. (FIG. 1C).

### 2-3. Confirmation of Pathological Damage to Tissues

H&E staining was performed to confirm pathological damage in tissue samples (brain, lung, and kidney) of K18-hDPP4 mice remaining after measuring the virus titers in Example 2-2. Specifically, each tissue was fixed with neutralizing buffered formalin (10%) and then embedded in paraffin. Each tissue was sectioned into 5 μm slices and stained with a hematoxylin (H) solution for 90 seconds. The tissues were washed with water, and then stained with an eosin (E) solution for 90 seconds and photographed with an Olympus DP70 microscope (Olympus Corporation, Tokyo, Japan).

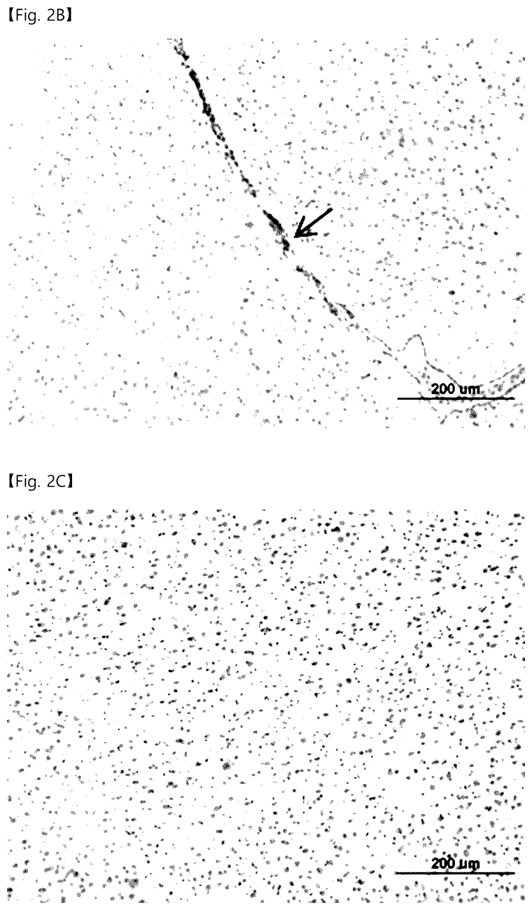

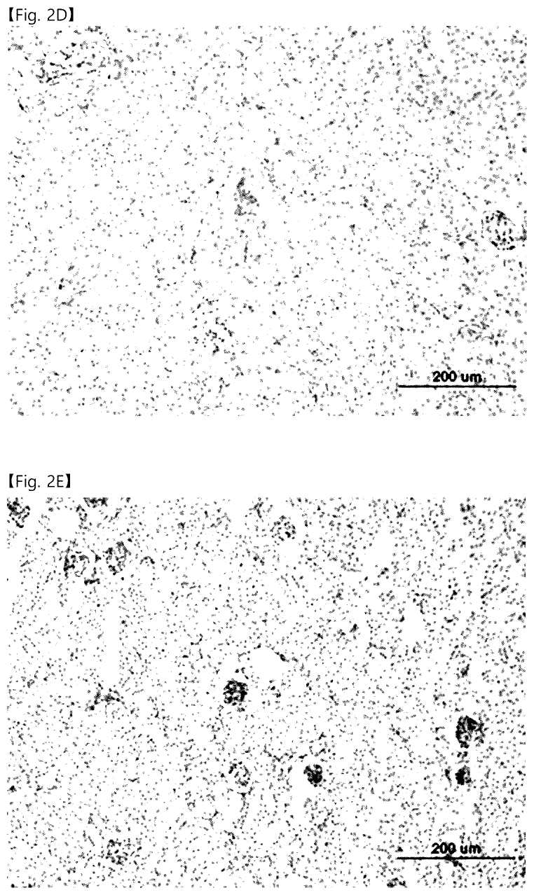

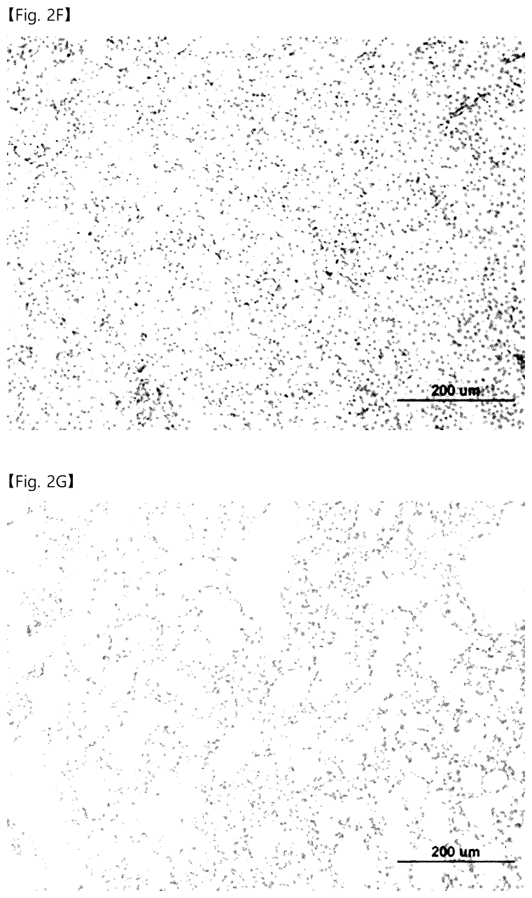

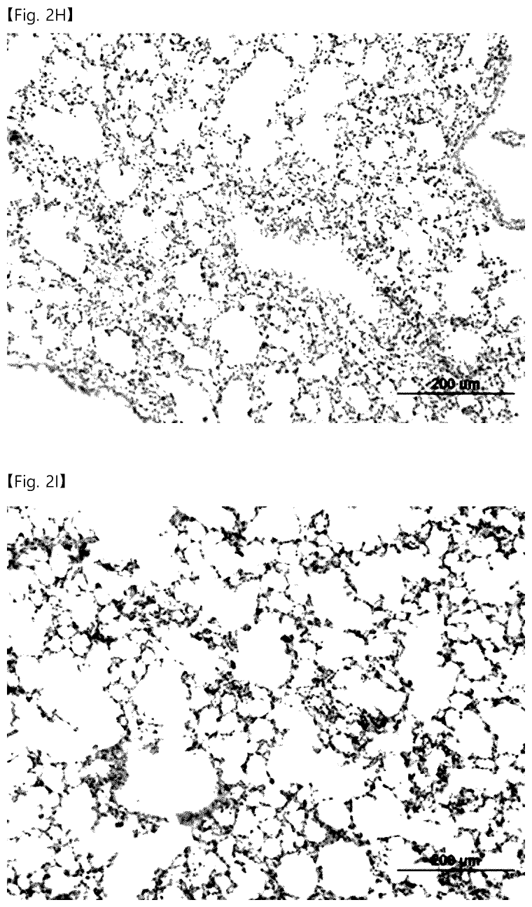

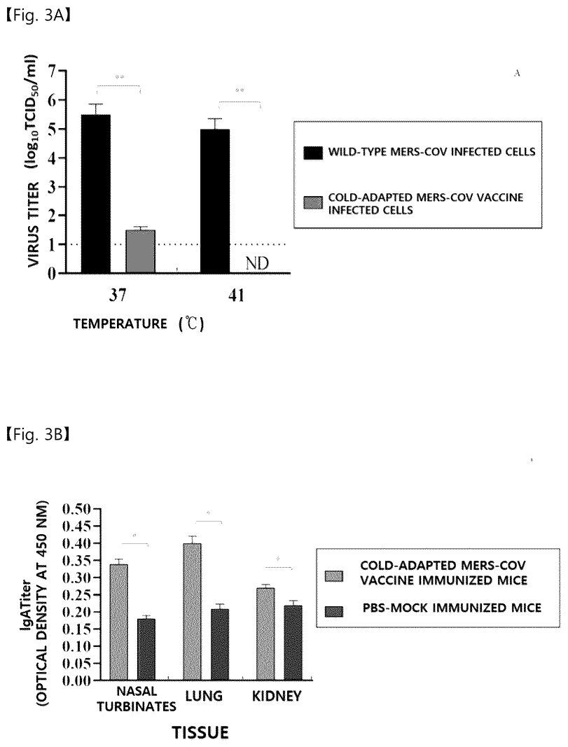

As a result, perivascular lymphocyte cuffing was observed in the brain of K18-hDPP4 mice infected with the wild-type MERS-CoV strain EMC2012 (FIG. 2B), whereas perivascular lymphocyte cuffing was not found in the brains of PBS-mock mice and K18-hDPP4 mice infected with the cold-adapted attenuated MERS-CoV vaccine strain EMC2012-CA22° C. (FIGS. 2A and 2C). In addition, interstitial lymphocytes were detected in the kidneys of mice infected with wild-type MERS-CoV (FIG. 2E), whereas there was no accumulation of lymphocyte infiltration in the kidneys of the PBS-mock mouse group or the mouse group infected with EMC2012-CA22° C. (FIGS. 2D and 2F). In addition, interstitial pneumonia involved with the lymphocyte infiltration was observed in the lungs of the mouse group infected with wild-type MERS-CoV (FIG. 2H), whereas pneumonia was not observed in the lungs of the PBS-mock mouse group or the mouse group infected with EMC2012-CA22° C. (FIGS. 2G and 2I).

### Example 3. Confirmation of Temperature Sensitivity of Cold-Adapted Attenuated MERS-CoV Live Vaccine Strain

The temperature sensitivity of the cold-adapted attenuated MERS-CoV vaccine strain EMC2012-CA22° C. of the present invention was confirmed as log₁₀TCID₅₀/ml at 37° C. and 41° C. in Vero cells. Specifically, the Vero cells were cultured in a 6-well plate and then infected with the live attenuated vaccine strain EMC2012-CA22° C. of the present invention and the wild-type MERS-CoV EMC2012 at 0.001 multiplicity of infections (m.o.i), respectively. The infected cells were cultured for 4 days in a 5% CO₂ incubator at a temperature of 37° C. or 41° C., and then the virus supernatant was serially diluted 10-fold in MEM supplemented with 1.5% BSA. The virus supernatant was infected into the Vero cells cultured in a 96-well plate (5% CO₂, 35° C.) and cultured for 4 days, and based on CPE observed under a microscope, the virus titer was calculated as log₁₀TCID₅₀/ml (log 10 tissue culture infectious dose 50/ml) at 35° C.

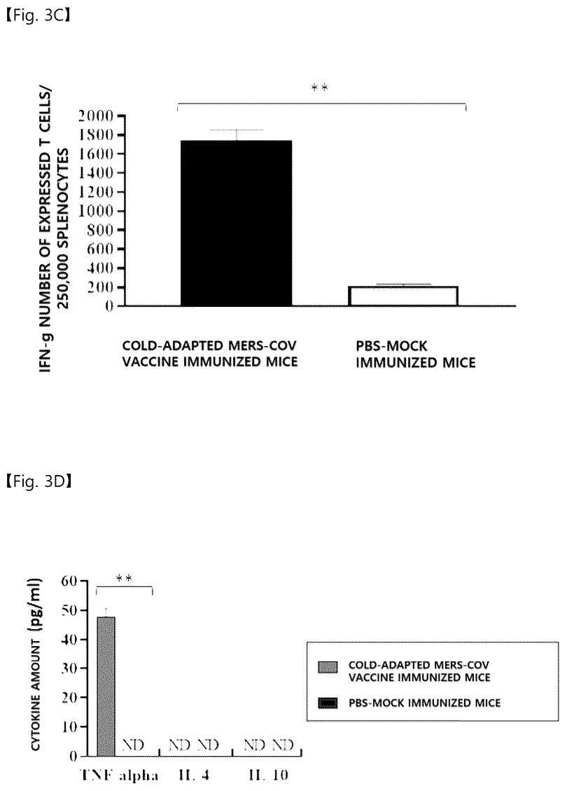

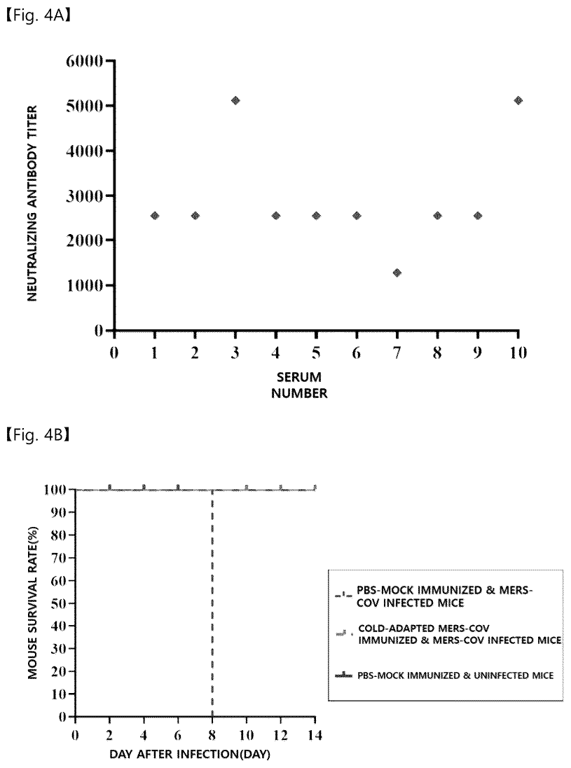

As a result, at 37° C., the virus titers of the cold-adapted attenuated MERS-CoV vaccine strain EMC2012-CA22° C. and the wild-type MERS-CoV EMC2012 were 1.5 log₁₀TCID₅₀/ml and 5.5 log₁₀TCID₅₀/ml, respectively, and at 41° C., the virus titer of the wild-type MERS-CoV EMC2012 was 5.0 log₁₀TCID₅₀/ml, but the virus titer of the cold-adapted attenuated MERS-CoV vaccine strain EMC2012-CA22° C. was not detected (FIG. 3A).

### Example 4. Confirmation of Immunization Effect of Cold-Adapted Attenuated MERS-CoV Live Vaccine Strain

### 4-1. Induction of Mucosal Immune Responses

Since IgA antibodies were known to be involved in mucosal immune responses, in order to determine whether mucosal antibodies IgA were induced in the tissues of mice immunized with the cold-adapted attenuated MERS-CoV vaccine strain EMC2012-CA22° C. of the present invention, K18-hDPP4 mice (n=13) were immunized with 2×10⁴ pfu of EMC2012-CA22° C. and four weeks after vaccination, the mice were challenged intranasally (i.n.) with 2×10⁴ pfu of wild-type MERS-CoV (Korean MERS-CoV/2015). Four weeks after immunization and six days after infection, three mice per group were sacrificed to measure IgA antibody induction in tissues (nasal turbinates, lung, and kidney). At this time, PBS-mock vaccinated and infected mice (n=13) were used as a control group. For IgA antibody measurement, the wells of a Nunc-Immuno™ MicroWell™ 96 well plate (Sigma-Aldrich) were coated overnight at 4° C. with a solution in which a purified inactivated MERS-CoV EMC2012-CA22° C. antigen was diluted at 100 μg/mL with a coating buffer (carbonate-bicarbonate buffer, pH 9.6). The wells of the plate were washed twice with 400 μl of a washing buffer PBS (pH 7.4) containing 4% horse serum and 0.05% Tween 20 (Sigma), and then each well was blocked overnight at 4° C. with 400 μl of a blocking buffer containing PBS and 4% skim milk. The buffer was removed, and each well was added with 100 μl of the tissue supernatant (10-fold diluted with the blocking buffer) homogenized from the nasal turbinates, lung, and kidney tissues of the mice vaccinated above or the mice vaccinated with PBS-mock and incubated at room temperature for 1 hour. Each well was added with 100 μl of goat anti-mouse IgA cross-adsorbed secondary antibody HRP (Invitrogen, MA, USA) diluted at 1:5000 with the blocking buffer and incubated for 1 hour at room temperature. Each well was washed 4 times with the washing buffer, added with 100 μl of goat anti-mouse IgA cross-adsorbed secondary antibody HRP (Invitrogen, MA, USA) diluted at 1:5000 with the blocking buffer and incubated for 1 hour at room temperature. Each well was washed four times with the washing buffer and then added with 100 μl of a TMB ELISA substrate (MABTECH), and incubated for 30 minutes at room temperature. The reaction was terminated by adding 100 μl of ABTS® Peroxidase Stop Solution (KPL, MD, USA), and the reaction absorbance at 450 nm was measured with an iMARK™ Microplate Absorbance Reader (Bio-Rad, CA, USA).

As a result, MERS-CoV-specific IgA antibodies were detected in the nasal turbinates, lung, and kidney, and the tissue having the highest IgA antibody value was the lung (OD value=0.4) (FIG. 3B).

### 4-2. Induction of Cellular Immunity

In order to determine whether the cold-adapted attenuated MERS-CoV vaccine strain EMC2012-CA22° C. of the present invention may induce cellular immunity important to regulate viral infection in a host, the number of lymphocytes expressing IFN-γ was measured in the spleens of mice inoculated with EMC2012-CA22° C. Specifically, K18-hDPP4 mice (n=6) were intranasally (i.n.) vaccinated with 2×10⁴ pfu of EMC2012-CA22° C. and then euthanized after 4 weeks and the spleens were collected. The collected spleens were homogenized in PBS (pH 7.4), and then the splenocytes were collected with an overlay of HISTOPAQUE-1077 (Sigma) and centrifuged (30 min, 1500, 4° C.). For IFN-γ ELISpot analysis, the layer containing lymphocytes was obtained using a Mouse IFN-γ ELISpot^{Plus} kit (MABTECH, Nacka Strand, Sweden). The wells of the plate were washed four times with 200 μl of PBS (pH 7.4) and stabilized with 200 μl of an RPMI 1640 medium supplemented with 10% FBS for 30 minutes at room temperature. Thereafter, prepared lymphocytes (250,000/well) infected with EMC2012-CA22° C. (0.01 m.o.i) were added to each well and incubated in an incubator (5% CO₂, 37° C.) for 24 hours. The wells were washed five times with 200 μl of PBS (pH 7.4), and added with a detection antibody R4-6A2-biotin diluted with 1 g/mL of PBS (pH 7.4) supplemented with 0.5% FBS (200 μl/well) and incubated for 2 hours at room temperature. The wells were washed five times with 200 μl of PBS (pH 7.4), and then 100 μl of streptavidin-ALP diluted at 1:1000 with PBS containing 0.5% FBS was added to each well. The plate was incubated at room temperature for 1 hour, and then washed 5 times with 200 μl of PBS (pH 7.4), and each well was added with 100 μl of a substrate solution (BCIP/NBT-plus) and developed until a clear spot was detected. Spots were counted under a microscope (Olympus).

As a result, the number of lymphocytes expressing IFN-γ was 1750/250,000 in the spleens of mice immunized with the EMC2012-CA22° C. vaccine strain (2×10⁴ pfu), and 220/250,000 in mice vaccinated with PBS-mock (FIG. 3C).

### 4-3. Measurement of Cytokine Amount

Splenocytes (2×10⁶/ml) of mice immunized with the MERS-CoV vaccine strain EMC2012-CA22° C. used for mouse IFN-γ ELISpot analysis in Example 4-2 were stimulated with EMC2012-CA22° C. (0.01 m.o.i) for 24 hours in an incubator and then a supernatant was collected and Th1 cytokine TNF-α and Th2 cytokines IL-4 and IL-10 were measured using ELISA kits for mice TNF-α, IL-4 and IL-10 (ThermoFisher SCIENTIIC, Waltham, MA, USA). Specifically, the supernatant or standard material (50 μl) was added to the wells of the plate coated with cytokine antibodies and incubated at room temperature for 2 hours. Thereafter, the wells were washed 6 times with 200 μl of a washing buffer, added with diluted Streptavidin-HRP (100 μl), and incubated at room temperature for 1 hour. The plate was washed with a washing buffer, added with a TMB substrate (100 μl), and incubated for 30 minutes at room temperature. The reaction was terminated by adding a stop solution (100 μl), the absorbance at 450 nm was measured using a spectrophotometer (Bio-Rad, Hercules, CA, USA), and the amount of cytokines was calculated based on a standard curve.

As a result, the Th1 cytokine, TNF-α, was found to be induced (48 pg/ml), but the Th2 cytokines IL-4 & IL-10 were not induced (FIG. 3D).

### Example 5. Confirmation of MERS-CoV Infection Prevention Effect of Cold-Adapted Attenuated MERS-CoV Live Vaccine Strain

5-1. Measurement of Neutralizing Antibody Titers after Immunization

In order to confirm a protective effect of the MERS-CoV vaccine strain EMC2012-CA22° C. of the present invention on MERS-CoV, K18-hDPP4 mice (n=13) were immunized intranasally (i.n.) with a single dose (2×10⁴ pfu) of EMC2012-CA22° C., and then the serum was collected after 4 weeks, and neutralizing antibody (NA) titers against Korean MERS-CoV/2015 were measured in Vero cells. Specifically, serum was collected from immunized mice (n=10 per group) at week 4 after vaccination with 2×10⁴ pfu of EMC2012-CA22° C., diluted 10-fold in MEM supplemented with 1.5% BSA, and serially twice diluted. The diluted serum (100 μl per sample) was incubated with 100 μl of wild-type SARS-CoV (Korean MERS-CoV/2015) at 100 TCID₅₀/mL in an incubator (5% CO₂, 37° C.) for 1 hour. Vero cells cultured in a 96-well plate were washed with warm PBS (pH 7.4) and inoculated with the serum sample mixed with the virus. The infected Vero cells were cultured for 4 days before CPE was observed. The titers of virus-neutralizing antibodies were determined as the reciprocal of the maximum dilution of serum in which infectivity of cells was 100% neutralized in a 96-well plate.

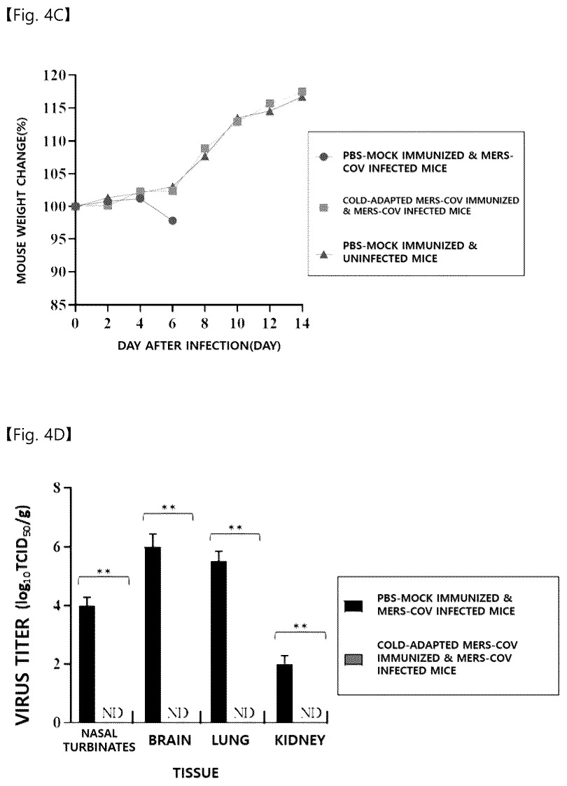

As a result, robust NA titers (1280 to 5120) were induced in immunized mice (FIG. 4A).

5-2. Confirmation of Pathogenicity During MERS-CoV Infection after Immunization

K18-hDPP4 mice (n=13 per group) were vaccinated with the MERS-CoV vaccine strain EMC2012-CA22° C. of the present invention, and after 4 weeks, infected with 2×10⁴ pfu of Korean MERS-CoV/2015, and then the mortality rate and weight changes of the mice were monitored for 14 days. As a control group, K18-hDPP4 mice (n=13) infected with PBS-mock were used.

As a result, all mice immunized and infected with EMC2012-CA22° C. survived without weight loss, while non-immunized mice died with 2.2% of weight loss on day 8 of infection (FIGS. 4B and 4C).

5-3. Measurement of Virus Titers in Tissues after Infection

K18-hDPP4 mice (n=3 per group) infected in Example 5-2 above were euthanized on day 6 of infection, and virus titers in tissues (nasal turbinates, brain, lung, and kidney) were measured.

As a result, the virus was not detected in the tissues of mice immunized and infected with EMC2012-CA22° C., whereas non-immunized and infected mice exhibited virus titers in tissues of 2.0 (kidney) to 6.0 (brain) TCID₅₀/g (FIG. 4D).

5-4. Confirmation of Pathological Damage to Tissues after Infection

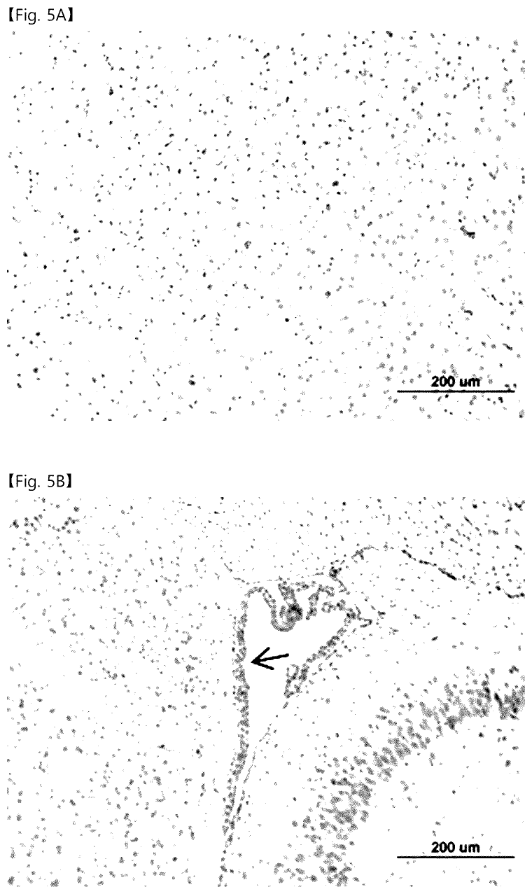

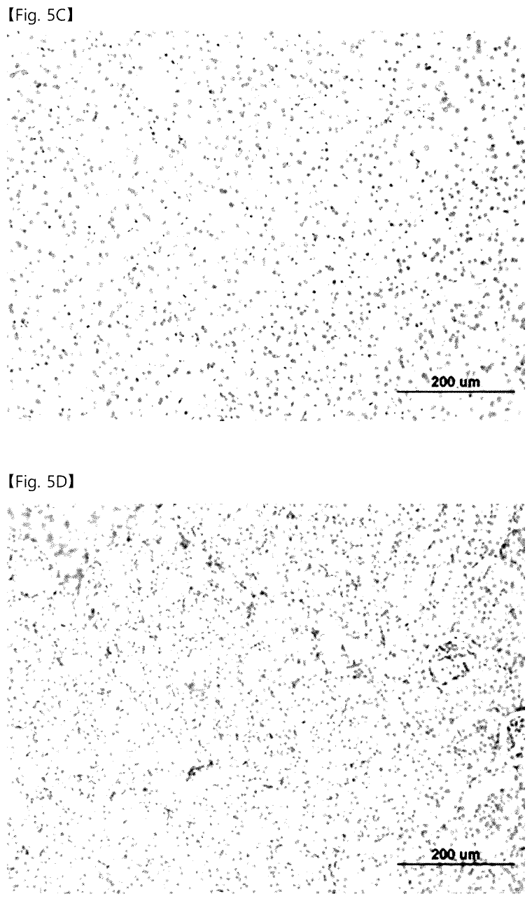

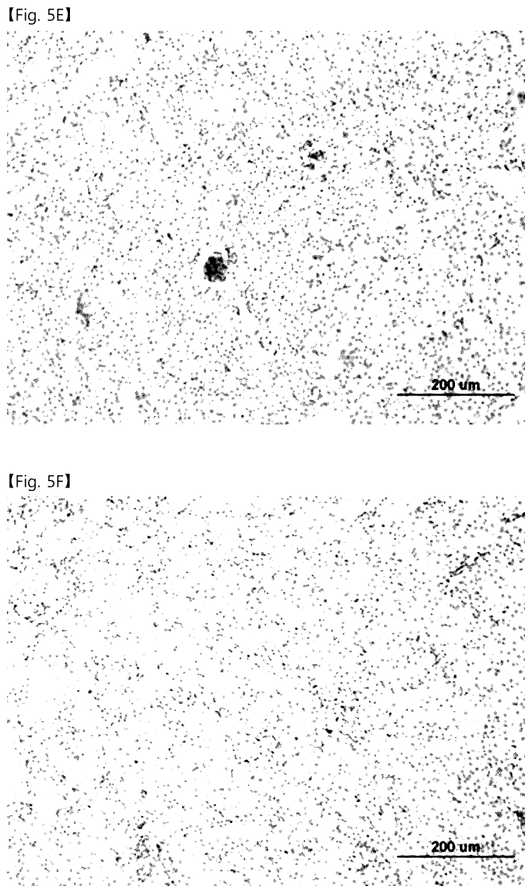

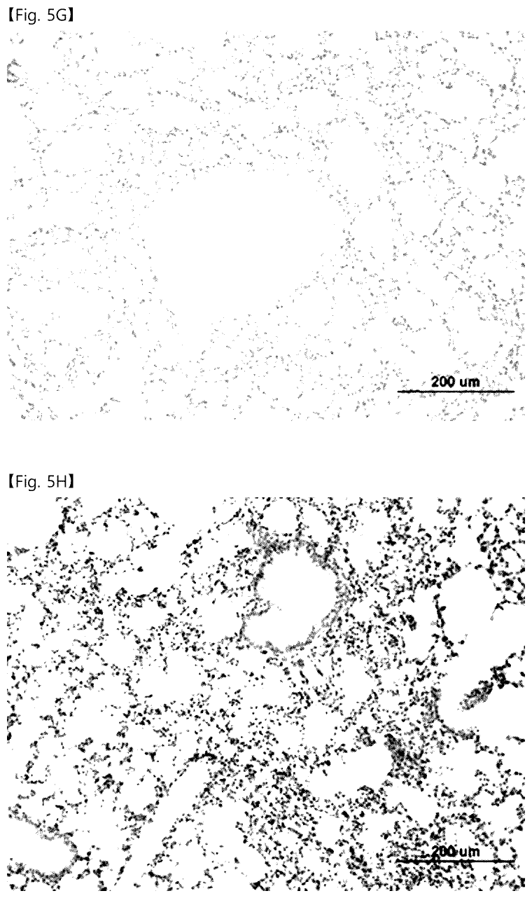

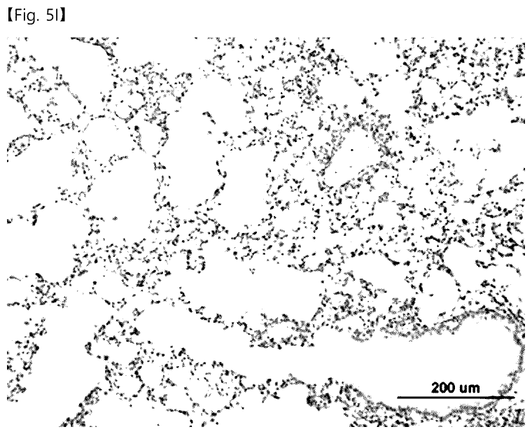

As a result of H&E staining the tissues (brain, lung and kidney) of the K18-hDPP4 mice infected in Example 5-2, perivascular lymphocyte cuffing was observed in the cerebral blood vessels of mice infected with wild-type MERS-CoV (FIG. 5B), but was not observed in the brains of mice immunized and infected with EMC2012-CA22° C. (FIG. 5C) or control mice (PBS-mock uninfected group) (FIG. 5A). In addition, infiltration of interstitial lymphocytes was observed in the kidneys of mice infected with wild-type MERS-CoV (FIG. 5E), but was not observed in the kidneys of mice immunized and infected with EMC2012-CA22° C. (FIG. 5F) or control mice (PBS-mock uninfected group) (FIG. 5d). In addition, interstitial pneumonia and lymphocyte infiltration were observed in the lung of mice infected with wild-type MERS-CoV (FIG. 5H), but pneumonia was not observed in mice immunized and infected with EMC2012-CA22° C. (FIG. 5I) or control mice (PBS-mock uninfected group) (FIG. 5G).

1. A cold-adapted attenuated Middle East respiratory syndrome coronavirus (MERS-CoV) comprising a gene encoding an amino acid in which at least amino acid selected from the group consisting of amino acids at positions 79, 1616, 2088, 2210, 3295, 3735, 3822, 4195, and 4247 in ORF1a polyprotein; positions 38, 66, 305, 872, 879, and 1251 in S protein; and position 5 in M protein of wild type MERS-CoV is substituted with a sequence different from a wild-type amino acid.

2. The cold-adapted attenuated MERS-CoV of claim 1, wherein the cold-adapted attenuated MERS-CoV includes a gene encoding an amino acid including at least one amino acid substitution selected from the group consisting of H79R, H1616L, T2088P, A2210V, Q3295R, F3735I, E3822G, P4195S, and N4247S in the ORF1a polyprotein; T38P, N66Y, S305R, T872A, I879T, and S1251F in the S protein; and T5M in the M protein.

3. The cold-adapted attenuated MERS-CoV of claim 1, wherein the ORF1a polyprotein of the wild-type MERS-CoV includes an amino acid sequence represented by SEQ ID NO: 3 or 4.

4. The cold-adapted attenuated MERS-CoV of claim 1, wherein the S protein of the wild-type MERS-CoV includes an amino acid sequence represented by SEQ ID NO: 5.

5. The cold-adapted attenuated MERS-CoV of claim 1, wherein the M protein of the wild-type MERS-CoV includes an amino acid sequence represented by SEQ ID NO: 11.

6. The cold-adapted attenuated MERS-CoV of claim 1, wherein the cold-adapted attenuated MERS-CoV includes a gene encoding the ORF1a polyprotein including an amino acid sequence represented by SEQ ID NO: 14 or 15.

7. The cold-adapted attenuated MERS-CoV of claim 1, wherein the cold-adapted attenuated MERS-CoV includes a gene encoding the S protein including an amino acid sequence represented by SEQ ID NO: 16.

8. The cold-adapted attenuated MERS-CoV of claim 1, wherein the cold-adapted attenuated MERS-CoV includes a gene encoding the M protein including an amino acid sequence represented by SEQ ID NO: 22.

9. The cold-adapted attenuated MERS-CoV of claim 1, wherein the wild-type MERS-CoV includes a nucleotide sequence represented by SEQ ID NO: 2.

10. The cold-adapted attenuated MERS-CoV of claim 1, wherein the cold-adapted attenuated MERS-CoV includes a nucleotide sequence represented by SEQ ID NO: 1.

11. The cold-adapted attenuated MERS-CoV of claim 1, wherein the cold-adapted attenuated MERS-CoV is prepared by infecting cells with the wild-type MERS-CoV and then adapting the cells gradually from 37° C. to 22° C.

12. The cold-adapted attenuated MERS-CoV of claim 11, wherein the cells are Vero cells, Calu-3, A549, HUHH7.0 or iEK-293T cells.

13. A vaccine composition for preventing infection with Middle East respiratory syndrome coronavirus (MERS-CoV), comprising the cold-adapted attenuated MERS-CoV of claim 1.

14. The vaccine composition for preventing infection with the MERS-CoV of claim 13, wherein the vaccine is a live vaccine.

15. The vaccine composition for preventing infection with the MERS-CoV of claim 13, wherein the vaccine composition is administered intranasally.

16. A pharmaceutical composition for preventing or treating Middle East respiratory syndrome (MERS) comprising the cold-adapted attenuated MERS-CoV of claim 1.

17. A method of preventing infection with Middle East respiratory syndrome coronavirus (MERS-CoV) by administering the vaccine composition of claim 13 to a subject.

18. A method of preventing or treating Middle East respiratory syndrome by administering the composition of claim 16 to a subject in need thereof.

19. A method for preparing cold-adapted attenuated Middle East respiratory syndrome coronavirus (MERS-CoV) comprising:
a) infecting cells with MERS-CoV and then adapting the cells gradually from 37° C. to 22° C.; and
b) subculturing the adapted MERS-CoV at 22° C. and then collecting the subcultured MERS-CoV.

20. The method of claim 19, wherein the adapting of the cells gradually from 37° C. to 22° C. is adapting the virus by lowering the temperature to the next lower temperature when the infected cells show a cytopathic effect (CPE).

21.-22. (canceled)


SEQUENCE LISTING (24 sequences)

SEQ ID NO: 1 - RNA, 30119 nt, unidentified
  source 1..30119: mol_type = genomic RNA
gatttaagtgaatagcttggctatcttacttcccctcgttctcttgcagaactttgattttaacgaacttaaataaaagccctgttgtttagcgtatcgttgcacttgtctggtgggattgtggcattaatttgcctgctcatctaggcagtggacatatgctcaacactgggtataattctaattgaatactatttttcagttagagcgtcgtgtctcttgtacgtctcggtcacaatacacggtttcgtccggtgcgtggcaattcggggcacatcatgtctttcgtggctggtgtgaccgcgcaaggtgcgcgcggtacgtatcgagcagcgctcaactctgaaaaacatcaagaccatgtgtctctaactgtgccactctgtggttcaggaaacctggttgaaaaactttcaccatggttcatggatggcgaaaatgcctatgaagtggtgaaggccatgttacttaaaaaggagccacttctctatgtgcccatccggctggctggacgcactagacacctcccaggtcctcgtgtgtacctggttgagaggctcattgcttgtgaaaatccattcatggttaaccaattggcttatagctctagtgcaaatggcagcctggttggcacaactttgcagggcaagcctattggtatgttcttcccttatgacatcgaacttgtcacaggaaagcaaaatattctcctgcgcaagtatggccgtggtggttatcactacaccccattccactatgagcgagacaacacctcttgccctgagtggatggacgattttgaggcggatcctaaaggcaaatatgcccagaatctgcttaagaagttgattggcggtgatgtcactccagttgaccaatacatgtgtggcgttgatggaaaacccattagtgcctacgcatttttaatggccaaggatggaataaccaaactggctgatgttgaagcggacgtcgcagcacgtgctgatgacgaaggcttcatcacattaaagaacaatctatatagattggtttggcatgttgagcgtaaagacgttccatatcctaagcaatctatttttactattaatagtgtggtccaaaaggatggtgttgaaaacactcctcctcactattttactcttggatgcaaaattttaacgctcaccccacgcaacaagtggagtggcgtttctgacttgtccctcaaacaaaaactcctttacaccttctatggtaaggagtcacttgagaacccaacctacatttaccactccgcattcattgagtgtggaagttgtggtaatgattcctggcttacagggaatgctatccaagggtttgcctgtggatgtggggcatcatatacagctaatgatgtcgaagtccaatcatctggcatgattaagccaaatgctcttctttgtgctacttgcccctttgctaagggtgatagctgttcttctaattgcaaacattcagttgctcagttggttagttacctttctgaacgctgtaatgttattgctgattctaagtccttcacacttatctttggtggcgtagcttacgcctactttggatgtgaggaaggtactatgtactttgtgcctagagctaagtctgttgtctcaaggattggagactccatctttacaggctgtactggctcttggaacaaggtcactcaaattgctaacatgttcttggaacagactcagcattcccttaactttgtgggagagttcgttgtcaacgatgttgtcctcgcaattctctctggaaccacaactaatgttgacaaaatacgccagcttctcaaaggtgtcacccttgacaagttgcgtgattatttagctgactatgacgtagcagtcactgccggcccattcatggataatgctattaatgttggtggtacaggattacagtatgccgccattactgcaccttatgtagttctcactggcttaggtgagtcctttaagaaagttgcaaccataccgtataaggtttgcaactctgttaaggatactctggcttattatgctcacagcgtgttgtacagagtttttccttatgacatggattctggtgtgtcatcctttagtgaactactttttgattgcgttgatctttcagtagcttctacctattttttagtccgcatcttgcaagataagactggcgactttatgtctacaattattacttcctgccaaactgctgttagtaagcttctagatacatgttttgaagctacagaagcaacatttaacttcttgttagatttggcaggattgttcagaatctttctccgcaatgcctatgtgtacacttcacaagggtttgtggtggtcaatggcaaagtttctacacttgtcaaacaagtgttagacttgcttaataagggtatgcaacttttgcatacaaaggtctcctgggctggttctaaaatcattgctgttatctacagcggcagggagtctctaatattcccatcgggaacctattactgtgtcaccactaaggctaagtccgttcaacaagatcttgacgttattttgcctggtgagttttccaagaagcagttaggactgctccaacctactgacaattctacaactgttagtgttactgtatccagtaacatggttgaaactgttgtgggtcaacttgagcaaactaatatgcatagtcctgatgttatagtaggtgactatgtcattattagtgaaaaattgtttgtgcgtagtaaggaagaagacggatttgccttctaccctgcttgcactaatggtcatgctgtaccgactctctttagacttaagggaggtgcacctgtaaaaaaagtagcctttggcggtgatcaagtacatgaggttgctgctgtaagaagtgttactgtcgagtacaacattcatgctgtattagacacactacttgcttcttctagtcttagaacctttgttgtagataagtctttgtcaattgaggagtttgctgacgtagtaaaggaacaagtctcagacttgcttgttaaattactgcgtggaatgccgattccagattttgatttagacgattttattgacgcaccatgctattgctttaacgctgagggtgatgcatcctggtcttctactatgatcttctctcttcaccccgtcgagtgtgacgaggagtgttctgaagtagaggcttcagatttagaagaaggtgaatcagagtgcatttctgagacttcaactgaacaagttgacgtttctcatgagacttctgacgacgagtgggctgctgcagttgatgaagcgttccctctcgatgaagcagaagatgttactgaatctgtgcaagaagaagcacaaccagtagaagtacctgttgaagatattgcgcaggttgtcatagctgacaccttacaggaaactcctgttgtgcctgatactgttgaagtcccaccgcaagtggtgaaacttccgtctgcacctcagactatccagcccgaggtaaaagaagttgcacctgtctatgaggctgataccgaacagacacagaatgttactgttaaacctaagaggttacgcaaaaagcgtaatgttgaccctttgtccaattttgaacataaggttattacagagtgcgttaccatagttttaggtgacgcaattcaagtagccaagtgctatggggagtctgtgttagttaatgctgctaacacacatcttaagcatggcggtggtatcgctggtgctattaatgcggcttcaaaaggggctgtccaaaaagagtcagatgagtatattctggctaaagggccgttacaagtaggagattcagttctcttgcaaggccattctctagctaagaatatcctgcatgtcgtaggcccagatgcccgcgctaaacaggatgtttctctccttagtaagtgctataaggctatgaatgcatatcctcttgtagtcactcctcttgtttcagcaggcatatttggtgtaaaaccagctgtgtcttttgattatcttattagggaggctaagactagagttttagtcgtcgttaattcccaagatgtctataagagtcttaccatagttgacattccacagagtttgactttttcatatgatgggttacgtggcgcaatacgtaaagctaaagattatggttttactgtttttgtgtgcacagacaactctgctaacactaaagttcttaggaacaagggtgttgattatactaagaagtttcttacagttgacggtgtgcaatattattgctacacgtctaaggacactttagatgatatcttacaacaggctaataagtctgttggtattatatctatgcctttgggatatgtgtctcatggtttagacttaatgcaagcagggagtgtcgtgcgtagagttaacgtgccctacgtgtgtctcctagctaataaagagcaagaagctattttgatgtctgaagacgttaagttaaacccttcagaagattttataaagcacgtccgcactaatggtggttacaattcttggcatttagtcgagggtgaactattggtgcaagacttacgcttaaataagctcctgcattggtctgatcaaaccatatgctacaaggatagtgtgttttatgttgtaaagaatagtacagcttttccatttgaaacactttcagcatgtcgtgcgtatttggattcacgcacgacacagcagttaacaatcgaagtcttagtgactgtcgatggtgtaaattttagaacagtcgttctaaataataagaacacttatagatcacagcttggatgcgttttctttaatggtgctgatatttctgacaccattcctgatgagaaacagaatggtcacagtttatatctagcagacaatttgactgctgatgaaacaaaggcgcttaaagagttatatggccccgttgatcctactttcttacacagattctattcacttaaggctgcagtccatgggtggaagatggttgtgtgtgataaggtacgttctctcaaattgagtgataataattgttatcttaatgcagttattatgacacttgatttattgaaggacattaaatttgttatacctgctctacagcttgcatttatgaaacataagggcggtgattcaactgacttcatagccctcattatggcttatggcaattgcacatttggtgctccagatgatgcctctcggttacttcataccgtgcttgcaaaggctgagttatgctgttctgcacgcatggtttggagagagtggtgcaatgtctgtggcataaaagatgttgttctacaaggcttaaaagcttgttgttacgtgggtgtgcaaactgttgaagatctgcgtgctcgcatgacatatgtatgccagtgtggtggtgaacgtcatcggcaattagtcgaacacaccaccccctggttgctgctctcaggcacaccaaatgaaaaattggtgacaacctccacggcgcctgattttgtagcatttaatgtctttcagggcattgaaacggctgttggccattatgttcatgctcgcctgaagggtggtcttattttaaagtttgactctggcaccgttagcaagacttcagactggaagtgcaaggtgacagatgtacttttccccggccaaaaatacagtagcgattgtaatgtcgtacggtattctttggacggtaatttcagaacagaggttgatcccgacctatctgctttctatgttaaggatggtaaatactttacaagtgaaccacccgtaacatattcaccagctacaattttagctggtagtgtctacactaatagctgccttgtatcgtctgatggacaacctggcggtgatgctattagtttgagttttaataaccttttagggtttgattctagtaaaccagtcactaagaaatacacttactccttcttgcctaaagaagacggcgatgtgttgttggctgagtttgacacttatgaccctatttataagaatggtgccatgtataaaggcaaaccaattctttgggtcaataaagcatcttatgatactaatcttaataagttcaatagagctagtttgcgtcaaatttttgacgtagcccccattgaactcgaaaataaattcacacctttgagtgtggagtctacaccagttgaacctccaactgtagatgtggtagcacttcaacaggaaatgacaattgtcaaatgtaagggtttaaataaacctttcgtgaaggacaatgtcagtttcgttgctgatgattcaggtactcccgttgttgagtatctgtctaaagaagacctacatacattgtatgtagaccctaagtatcaagtcattgtcttaaaagacaatgtactttcttctatgcttagattgcacaccgttgagtcaggtgatattaacgttgttgcagcttccggatctttgacacgtaaagtgaagttactatttagggcttcattttatttcaaagaatttgctacccgcactttcactgctacccctgctgtaggtagttgtataaagagtgtagtgcggcatctaggtgttactaaaggcatattgacaggctgttttagttttgccaagatgttatttatgcttccactagcttactttagtgattcaaaactcggcaccacagaggttaaagtgagtgctttgaaaacagccggcgttgtgacaggtaatgttgtaaaacagtgttgcactgctgctgttgatttaagtatggataagttgcgccgtgtggattggaaatcaaccctacggttgttacttatgttatgcacaactatggtattgttgtcttctgtgtatcacttgtatgtcttcaatcaggtcttatcaagtgatgttatgtttgaagatgtccaaggtttgaaaaagttctacaaagaagttagagcttacctaggaatctcttctgcttgtgacggtcttgcttcagcttatagggcgaattcctttgatgtacctacattctgcgcaaaccgttctgcaatgtgtaattggtgcttgattagccaagattccataactcactacccagctcttaagatggttcaaacacatcttagccactatgttcttaacatagattggttgtggtttgcatttgagactggtttggcatacatgctctatacctcggccttcaactggttgttgttggcaggtacattgcattatttctttgcacagacttccatatttgtagactggcggtcatacaattatgctgtgtctagtgccttctggttattcacccacattccaatggcgggtttggtacgaatgtataatttgttagcatgcctttggcttttacgcaagttttatcagcatgtaatcaatggttgcaaagatacggcatgcttgctctgctataagaggaaccgacttactagagttgaagcttctaccgttgtctgtggtggaaaacgtacgttttatatcacagcaaatggcggtatttcattctgtcgtaggcataattggaattgtgtggattgtgacactgcaggtgtggggaataccttcatctgtgaagaagtcgcaaatgacctcactaccgccctacgcaggcctattaacgctacggatagatcacattattatgtggattccgttacagttaaagagactgttgttcagtttaattatcgtagagacggtcaaccattctacgagcggtttcccctctgcgcttttacaaatctagataagttgaagttcaaagaggtctgtaaaactactactggtatacctgaatacaactttatcatctacgactcatcagatcgtggccaggaaagtttagctaggtctgcatgtgtttattattctcaagtcttgtgtaaatcaattcttttggttgactcaagtttggttacttctgttggtgattctagtgaaatcgccactaaaatgtttgattcctttgttaatagtttcgtctcgctgtataatgtcacacgcgataagttggaaaaacttatctctactgctcgtgatggcgtaaggcgaggcgataacttccatagtgtcttaacaacattcattgacgcagcacgaggccccgcaggtgtggagtctgatgttgagaccaatgaaattgttgactctgtgcagtatgctcataaacatgacatacaaattactaatgagagctacaataattatgtaccctcatatgttaaacctgatagtgtgtctaccagcgatttaggtagtctcattgattgtaatgcggcttcagttaaccaaattgtcttgcgtaattctaatggtgcttgcatttggaacgctgctgcatatatgaaactctcggatgcacttaaacgacagattcgcattgcatgccgtaagtgtaatttagctttccggttaaccacctcaaagctacgcgctaatgataatatcttatcagttagattcactgctaacaaaattgttggtggtgctcctacatggtttaatgcgttgcgtgactttacgttaaagggttatgttcttgctaccattattgtgtttctgtgtgctgtactgatgtatttgtgtttacctacattttctatggcacctgttgaattttatgaagaccgcatcttggactttaaagttcttgataatggtatcattagggatgtaaatcctgatgataagtgctttgctaataagcaccggtccttcacacaatggtatcatgagcatgttggtggtgtctatgacaactctatcacatgcccattgacagttgcagtaattgctggagttgctggtgctcgcattccagacgtacctactacattggcttgggtgaacaatcagataattttctttgtttctcgagtctttgctaatacaggcagtgtttgctacactcctatagatgagataccctataagagtttctctgatagtggttgcattcttccatctgagtgcactatgtttagggatgcagagggccgtatgacaccatactgccatgatcctactgttttgcctggggcttttgcgtacagtcagatgaggcctcatgttcgttacgacttgtatgatggtaacatgtttattaaatttcctgaagtagtatttgaaagtacacttaggattactagaactctgtcaactcagtactgccggttcggtagttgtgagtatgcacaagagggtgtttgtattaccacaaatggctcgtgggccatttttaatgaccaccatcttaatagacctggtgtctattgtggctctgattttattgacattgtcaggcggttagcagtatcactgttccagcctattacttatttccaattgactacctcattggtcttgggtataggtttgtgtgcgttcctgactttgctcttctattatattaataaagtaaaacgtgcttttgcagattacacccagtgtgctgtaattgctgttgttgctgctgttcttaatagcttgtgcatctgctttgttacctctataccattgtgtatagtaccttacactgcattgtactattatgctacattctattttactaatgagcctgcatttattatgcatgtttcttggtacattatgttcgggcctatcgttcccatatggatgacctgcgtctatacagttgcaatgtgctttagacacttcttctgggttttagcttattttagtaagaaacatgtagaagtttttactgatggtaagcttaattgtagtttccaggacgctgcctctaatatctttgttattaacaaggacacttatgcagctcttagaaactctttaactaatgatgcctattcacgatttttggggttgtttaacaagtataagtacttctctggtgctatggaaacagccgcttatcgtgaagctgcagcatgtcatcttgctaaagccttacaaacatacagcgagactggtagtgatcttctttaccaaccacccaactgtagcataacctctggcgtgttgcaaagcggtttggtgaaaatgtcacatcccagtggagatgttgaggcttgtatggttcaggttacctgcggtagcatgactcttaatggtctttggcttgacaacacagtctggtgcccacgacacgtaatgtgcccggctgaccggttgtctgatcctaattatgatgccttgttgatttctatgactaatcatagtttcagtgtgcaaaaacacattggcgctccagcaaacttgcgtgttgttggtcatgccatgcaaggcactcttttgaagttgactgtcgatgttgctaaccctagcactccagcctacacttttacaacagtgaaacctggcgcagcatttagtgtgttagcatgctataatggtcgtccgactggtacattcactgttgtaatgcgccctaactacacaattaagggttcctttctgtgtggttcttgtggtagtgttggttacaccaaggagggtagtgtgatcaatttctgttacatgcatcaaatggaacttgctaatggtacacataccggttcagcatttgatggtactatgtatggtgcctttatggataaacaagtgcaccaagttcagttaacagacaaatactgcagtgttaatgtagtagcttggctttacgcagcaatacttaatggttgcgcttggtttgtaaaacctaatcgcactagtgttgtttcttttaatgaatgggctcttgccaaccaattcactgaatttgttggcactcaatccgttgacatgttagctgtcaaaacaggcgttgctattgaacagctgctttatgcgatccaacaactgtatactgggttccagggaaagcaaatccttggcagtaccatgttggaagatgaattcacacctgaggatgttaatatgcagattatgggtgtggttatgcagagtggtgtgagaaaagttacatatggtactgcgcattggttgtttgcgacccttgtctcaacctatgtgataatcttacaagccactaaatttactttgtggaactacttgtttgagactattcccacacagttgttcccactcttatttgtgactatggccttcgttatgttgttggttaaacacaaacacacctttttgacacttttcttgttgcctgtggctatttgtttgacttatgcaaacatagtctacgagcccactactcccatttcgtcagcgctgattgcagttgcaaattggcttgcccccactaatgcttatatgcgcactacacatactgatattggtgtctacattagtatgtcacttgtattagtcattgtagtgaagagattgtacaacccatcactttctaactttgcgttagcattgtgcagtggtgtaatgtggttgtacacttatagcattggagaagcctcaagccccattgcctatctggtttttgtcactacactcactagtgattatacgattacagtcattgttactgtcaaccttgcaaaagtttgcacttatgccatctttgcttactcaccacagcttacacttgtgtttccggaagtgaagatgatacttttattatacacatgtttaggtttcatgtgtacttgctattttggtgtcttctctcttttgaaccttaagcttagagcacctatgggtgtctatgactttaaggtctcaacacaagagttcagattcatgactgctaacaatctaactgcacctagaaattcttggggggctatggctctgaactttaagttaataggtattggcggtacaccttgtataaaggttgctgctatgcagtctaaacttacagatcttaaatgcacatctgtggttctcctctctgtgctccaacagttacacttagaggctaatagtagggcctgggctttctgtgttaaatgccataatgatatattggcagcaacagaccccagtgaggctttcgagaaattcgtaagtctctttgctactttaatgactttttctggtaatgtagatcttgatgcgttagctagtgatatttttgacactcctagcgtacttcaagctactctttctgagttttcacacttagctacctttgctgagttggaagctgcgcagaaagcctatcaggaagctatggactctggtgacacctcaccacaagttcttaaggctttgcagaaggctgttaatatagctaaaaacgcctatgagaaggataaggcagtggcccgtaagttagaacgtatggctgatcaggctatgacttctatgtataagcaagcacgtgctgaagacaagaaagcaaaaattgtcagtgctatgcaaactatgttgtttggtatgattaagaagctcgacaacgatgttcttaatggtatcatttctaacgctaggaatggttgtatacctcttagtgtcatcccactgtgtgcttcaaataaacttcgcgttgtaattcctgacttcaccgtctggaatcaggtagtcacatatccctcgcttaactacgctggggctttgtgggacattacagttataaacaatgtggacaatgaaattgttaagtcttcagatgttgtagacagcaatgaaaatttaacatggccacttgttttagaatgcactagggcatccacttctgccgttaagttgcaaaataatgagatcaaaccttcaggtctaaaaaccatggttgtgtctgcgggtcaagagcaaactaactgtaatactagttccttagcttattacgaacctgtgcagggtcgtaaaatgctgatggctcttctttctgataatgcctatctcaaatgggcgcgtgttgaaggtaaggacggatttgtcagtgtagagctacaatctccttgcaaattcttgattgcgggaccaaaaggacctgaaatccgatatctctattttgttaaaaatcttaacaaccttcatcgcgggcaagtgttagggcacattgctgcgactgttagattgcaagctggttctaacaccgagtttgcctctagttcctcggtgttgtcacttgttaacttcaccgttgatcctcaaaaagcttatctcgatttcgtcaatgcgggaggtgccccattgacaaattgtgttaagatgcttactcctaaaactggtacaggtatagctatatctgttaaaccagagagtacagctgatcaagagacttatggtggagcttcagtgtgtctctattgccgtgcgcatatagaacatcctgatgtctctggtgtttgtaaatataagggtaagtttgtccaaatccctgctcagtgtgtccgtgaccctgtgggattttgtttgtcaaataccccctgtaatgtctgtcaatattggattggatatgggtgcaattgtgactcgcttaggcaagcagcactgccccaatctaaagattccaattttttaaacgagtccggggttctattgtaaatgcccgaatagaaccctgttcaagtggtttgtccactgatgtcgtctttagggcatttgacatctgcaactataaggctaaggttgctggtattggaaaatactacaagactaatacttgtaggtttgtagaattagatgaccaagggcatcatttagactcctattttgtcgttaagaggcatactatggagaattatgaactagagaagcactgttacgacttgttacgtgactgtgatgctgtagctccccatgatttcttcatctttgatgtagacaaagttaaaacacctcatattgtacgtcagcgtttaactgagtacactatgatggatcttgtatatgccctgaggcactttgatcaaaatagcgaagtgcttaaggctatcttagtgaagtatggttgctgtgatgttacctactttgaaaataaactctggtttgattttgttgaaaatcccagtgttattggtgtttatcataaacttggagaacgtgtacgccaagctatcttaaacactgttaaattttgtgaccacatggtcaaggctggtttagtcggtgtgctcacactagacaaccaggaccttaatggcaagtggtatgattttggtgacttcgtaatcactcaacctggttcaggagtagctatagttgatagctactattcttatttgatgcctgtgctctcaatgaccgattgtctggccgctgagacacatagggattgtgattttaataaaccactcattgagtggccacttactgagtatgattttactgattataaggtacaactctttgagaagtactttaaatattgggatcagacgtatcacgcaaattgcgttaattgtactgatgaccgttgtgtgttacattgtgctaatttcaatgtattgtttgctatgaccatgcctaagacttgtttcggacccatagtccgaaagatctttgttgatggcgtgccatttgtagtatcttgtggttatcactacaaagaattaggtttagtcatgaatatggatgttagtctccatagacataggctctctcttaaggagttgatgatgtatgccgctgatccagccatgcacattgcctcctctaacgcttttcttgatttgaggacatcatgttttagtgtcgctgcacttacaactggtttgacttttcaaactgtgcggcctggcaattttaaccaagacttctatgatttcgtggtatctaaaggtttctttaaggagggctcttcagtgacgctcaaacattttttctttgctcaagatggtaatgctgctattacagattataattactattcttataatctgcctactatgtgtgacatcaaacaaatgttgttctgcatggaagttgtaaacaagtacttcgaaatctatgacggtggttgtcttaatgcttctgaagtggttgttaataatttagacaagagtgctggccatccttttaataagtttggcaaagctcgtgtctattatgagagcatgtcttaccaggagcaagatgaactttttgccatgacaaagcgtaacgtcattcctaccatgactcaaatgaatctaaaatatgctattagtgctaagaatagagctcgcactgttgcaggcgtgtccatacttagcacaatgactaatcgccagtaccatcagaaaatgcttaagtccatggctgcaactcgtggagcgacttgcgtcattggtactacaaagttctacggtggctgggatttcatgcttaaaacattgtacaaagatgttgataatccgcatcttatgggttgggattaccctaagtgtgatagagctatgcctaatatgtgtagaatcttcgcttcactcatattagctcgtaaacatggcacttgttgtactacaagggacagattttatcgcttggcaaatgagtgtgctcaggtgctaagcgaatatgttctatgtggtggtggttactacgtcaaacctggaggtaccagtagcggagatgccaccactgcatatgccaatagtgtctttaacattttgcaggcgacaactgctaatgtcagtgcacttatgggtgctaatggcaacaagattgttgacaaagaagttaaagacatgcagtttgatttgtatgtcaatgtttacaggagcactagcccagaccccaaatttgttgataaatactatgcttttcttaataagcacttttctatgatgatactgtctgatgacggtgtcgtttgctataatagtgattatgcagctaagggttacattgctggaatacagaattttaaggaaacgctgtattatcagaacaatgtctttatgtctgaagctaaatgctgggtggaaaccgatctgaagaaagggccacatgaattctgttcacagcatacgctttatattaaggatggcgacgatggttacttccttccttatccagacccttcaagaattttgtctgccggttgctttgtagatgatatcgttaagactgacggtacactcatggtagagcggtttgtgtctttggctatagatgcttaccctctcacaaagcatgaagatatagaataccagaatgtattctgggtctacttacagtatatagaaaaactgtataaagaccttacaggacacatgcttgacagttattctgtcatgctatgtggtgataattctgctaagttttgggaagaggcattctatagagatctctatagttcgcctaccactttgcaggctgtcggttcatgcgttgtatgccattcacagacttccctacgctgtgggacatgcatccgtagaccatttctctgctgtaaatgctgctatgatcatgttatagcaactccacataagatggttttgtctgtttctccttacgtttgtaatgcccctggttgtggcgtttcagacgttactaagctatatttaggtggtatgagctacttttgtgtagatcatagacctgtgtgtagttttccactttgcgctaatggtcttgtattcggcttatacaagaatatgtgcacaggtagtccttctatagttgaatttaataggttggctacctgtgactggactgaaagtggtgattacacccttgccaatactacaacagaaccactcaaactttttgctgctgagactttacgtgccactgaagaggcgtctaagcagtcttatgctattgccaccatcaaagaaattgttggtgagcgccaactattacttgtgtgggaggctggcaagtccaaaccaccactcaatcgtaattatgtttttactggttatcatataaccaaaaatagtaaagtgcagctcggtgagtacattttcgagcgcattgattatagtgatgctgtatcctacaagtctagtacaacgtataaactgactgtaggtgacatcttcgtacttacctctcactctgtggctaccttgacggcgcccacaattgtgaatcaagagaggtatgttaaaattactgggttgtacccaaccattacggtacctgaagagttcgcaagtcatgttgccaacttccaaaaatcaggttatagtaaatatgtcactgttcagggaccacctggcactggcaaaagtcattttgctatagggttagcgatttactaccctacagcacgtgttgtttatacagcatgttcacacgcagctgttgatgctttgtgtgaaaaagcttttaaatatttgaacattgctaaatgttcccgtatcattcctgcaaaggcacgtgttgagtgctatgacaggtttaaagttaatgagacaaattctcaatatttgtttagtactattaatgctctaccagaaacttctgccgatattctggtggttgatgaggttagtatgtgcactaattatgatctttcaattattaatgcacgtattaaagctaagcacattgtctatgtaggagatccagcacagttgccagctcctaggactttgttgactagaggcacattggaaccagaaaatttcaatagtgtcactagattgatgtgtaacttaggtcctgacatatttttaagtatgtgctacaggtgtcctaaggaaatagtaagcactgtgagcgctcttgtctacaataataaattgttagccaagaaggagctttcaggccagtgctttaaaatactctataagggcaatgtgacgcatgatgctagctctgccattaatagaccacaactcacatttgtgaagaattttattactgccaatccggcatggagtaaggcagtctttatttcgccttacaattcacagaatgctgtgtctcgttcaatgctgggtcttaccactcagactgttgattcctcacagggttcagaataccagtacgttatcttctgtcaaacagcagatacggcacatgctaacaacattaacagatttaatgttgcaatcactcgtgcccaaaaaggtattctttgtgttatgacatctcaggcactctttgagtccttagagtttactgaattgtcttttactaattacaagctccagtctcagattgtaactggcctttttaaagattgctctagagaaacttctggcctctcacctgcttatgcaccaacatatgttagtgttgatgacaagtataagacgagtgatgagctttgcgtgaatcttaatttacccgcaaatgtcccatactctcgtgttatttccaggatgggctttaaactcgatgcaacagttcctggatatcctaagcttttcattactcgtgaagaggctgtaaggcaagttcgaagctggataggcttcgatgttgagggtgctcatgcttcccgtaatgcatgtggcaccaatgtgcctctacaattaggattttcaactggtgtgaactttgttgttcagccagttggtgttgtagacactgagtggggtaacatgttaacgggcattgctgcacgtcctccaccaggtgaacagtttaagcacctcgtgcctcttatgcataagggggctgcgtggcctattgttagacgacgtatagtgcaaatgttgtcagacactttagacaaattgtctgattactgtacgtttgtttgttgggctcatggctttgaattaacgtctgcatcatacttttgcaagataggtaaggaacagaagtgttgcatgtgcaatagacgcgctgcagcgtactcttcacctctgcaatcttatgcctgctggactcattcctgcggttatgattatgtctacaaccctttctttgtcgatgttcaacagtggggttatgtaggcaatcttgctactaatcacgatcgttattgctctgtccatcaaggagctcatgtggcttctaatgatgcaataatgactcgttgtttagctattcattcttgttttatagaacgtgtggattgggatatagagtatccttatatctcacatgaaaagaaattgaattcctgttgtagaatcgttgagcgcaacgtcgtacgtgctgctcttcttgccggttcatttgacaaagtctatgatattggcaatcctaaaggaattcctattgttgatgaccctgtggttgattggcattattttgatgcacagcccttgaccaggaaggtacaacagcttttctatacagaggacatggcctcaagatttgctgatgggctctgcttattttggaactgtaatgtaccaaaatatcctaataatgcaattgtatgcaggtttgacacacgtgtgcattctgagttcaatttgccaggttgtgatggcggtagtttgtatgttaacaagcacgcttttcatacaccagcatatgatgtgagtgcattccgtgatctgaaacctttaccattcttttattattctactacaccatgtgaagtgcatggtaatggtagtatgatagaggatattgattatgtacccctaaaatctgcagtctgtattacagcttgtaatttagggggcgctgtttgtaggaagcatgctacagagtacagagagtatatggaagcatataatcttgtctctgcatcaggtttccgcctttggtgttataagacctttgatatttataatctctggtctacttttacaaaagttcaaggtttggaaaacattgcttttaatgttgttaaacaaggccattttattggtgttgagggtgaactacctgtagctgtagtcaatgataagatcttcaccaagagtggcgttaatgacatttgtatgtttgagaataaaaccactttgcctactaatatagcttttgaactctatgctaagcgtgctgtacgctcgcatcccgatttcaaattgctacacaatttacaagcagacatttgctacaagttcgtcctttgggattatgaacgtagcaatatttatggtactgctactattggtgtatgtaagtacactgatattgatgttaattcagctttgaatatatgttttgacatacgcgataattgttcattggagaagttcatgtctactcccaatgccatctttatttctgatagaaaaatcaagaaatacccttgtatggtaggtcctgattatgcttacttcaatggtgctatcatccgtgatagtgatgttgttaaacaaccagtgaagttctacttgtataagaaagtcaataatgagtttattgatcctactgagtgtatttacactcagagtcgctcttgtagtgacttcctacccctttctgacatggagaaagactttctatcttttgatagtgatgttttcattaagaagtatggcttggaaaactatgcttttgagcacgtagtctatggagacttctctcatactacgttaggcggtcttcacttgcttattggtttatacaagaagcaacaggaaggtcatattattatggaagaaatgctaaaaggtagctcaactattcataactattttattactgagactaacacagcggcttttaaggcggtgtgttctgttatagatttaaagcttgacgactttgttatgattttaaagagtcaagaccttggcgtagtatccaaggttgtcaaggttcctattgacttaacaatgattgagtttatgttatggtgtaaggatggacaggttcaaaccttctaccctcgactccaggcttctgcagattggaaacctggtcatgcaatgccatccctctttaaagttcaaaatgtaaaccttgaacgttgtgagcttgctaattacaagcaatctattcctatgcctcgcggtgtgcacatgaacatcgctaaatatatgcaattgtgccagtatttaaatacttgcacattagccgtgcctgccaatatgcgtgttatacattttggcgctggttctgataaaggtatcgctcctggtacctcagttttacgacagtggcttcctacagatgccattattatagataatgatttaaatgagttcgtgtcagatgctgacataactttatttggagattgtgtaactgtacgtgtcggccaacaagtggatcttgttatttccgacatgtatgatcctactactaagaatgtaacaggtagtaatgagtcaaaggctttattctttacttacctgtgtaacctcattaataataatcttgctcttggtgggtctgttgctattaaaataacagaacactcttggagcgttgaactttatgaacttatgggaaaatttgcttggtggactgttttctgcaccaatgcaaatgcatcctcatctgaaggattcctcttaggtattaattacttgggtactattaaagaaaatatagatggtggtgctatgcacgccaactatatattttggagaaattccactcctatgaatctgagtacttactcactttttgatttatccaagtttcaattaaaattaaaaggaacaccagttcttcaattaaaggagagtcaaattaacgaactcgtaatatctctcctgtcgcagggtaagttacttatccgtgacaatgatacactcagtgtttctactgatgttcttgttaacacctacagaaagttacgttgatgtagggccagattctgttaagtctgcttgtattgaggttgatatacaacagcctttctttgataaaacttggcctaggccaattgatgtttctaaggctgacggtattatataccctcaaggccgtacatattcttacataactatcacttatcaaggtctttttccctatcagggagaccatggtgatatgtatgtttactctgcaggacatgctacaggcacaactccacaaaagttgtttgtagctaactattctcaggacgtcaaacagtttgctaatgggtttgtcgtccgtataggagcagctgccaattccactggcactgttattattagcccatctaccagcgctactatacgaaaaatttaccctgcttttatgctgggttcttcagttggtaatttctcagatggtaaaatgggccgcttcttcaatcatactctagttcttttgcccgatggatgtggcactttacttagagctttttattgtattctagagcctcgctctggaaatcattgtcctgctggcaattcctatacttcttttgccacttatcacactcctgcaacagattgttctgatggcaattacaatcgtaatgccagtctgaactcttttaaggagtattttaatttacgtaactgcacctttatgtacacttataacattaccgaagatgagattttagagtggtttggcattacacaaactgctcaaggtgttcacctcttctcatctcggtatgttgatttgtacggcggcaatatgtttcaatttgccaccttgcctgtttatgatactattaagtattattctatcattcctcacagtattcgttctatccaacgtgatagaaaagcttgggctgccttctacgtatataaacttcaaccgttaactttcctgttggatttttctgttgatggttatatacgcagagctatagactgtggttttaatgatttgtcacaactccactgctcatatgaatccttcgatgttgaatctggagtttattcagtttcgtctttcgaagcaaaaccttctggctcagttgtggaacaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcagcaattgctagcaactgttattcttcactgattttggattacttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaacaagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagcttgaatttgctaatgacacaaaaattgcctctcaattaggcaattgcgtggaatattccctctatggtgtttcgggccgtggtgtttttcagaattgcacagctgtaggtgttcgacagcagcgctttgtttatgatgcgtaccagaatttagttggctattattctgatgatggcaactactactgtttgcgtgcttgtgttagtgttcctgtttctgtcatctatgataaagaaactaaaacccacgctactctatttggtagtgttgcatgtgaacacatttcttctaccatgtctcaatactcccgttctacgcgatcaatgcttaaacggcgagattctacatatggcccccttcagacacctgttggttgtgtcctaggacttgttaattcctctttgttcgtagaggactgcaagttgcctcttggtcaatctctctgtgctcttcctgacacacctagtactctcacacctcgcagtgtgcgctctgttccaggtgaaatgcgcttggcatccattgcttttaatcatcctattcaggttgatcaacttaatagtagttattttaaattaagtatacccactaatttttcctttggtgtgactcaggagtacattcagacaaccattcagaaagttactgttgattgtaaacagtacgtttgcaatggtttccagaagtgtgagcaattactgcgcgagtatggccagttttgttccaaaataaaccaggctctccatggtgccaatttacgccaggatgattctgtacgtaatttgtttgcgagcgtgaaaagctctcaatcatctcctatcataccaggttttggaggtgactttaatttggcacttctagaacctgtttctacatctactggcagtcgtagtgcacgtagtgctattgaggatttgctatttgacaaagtcactatagctgatcctggttatatgcaaggttacgatgattgcatgcagcaaggtccagcatcagctcgtgatcttatttgtgctcaatatgtggctggttacaaagtattacctcctcttatggatgttaatatggaagccgcgtatacttcatctttgcttggcagcatagcaggtgttggctggactgctggcttatcctcctttgctgctattccatttgcacagagtatcttttataggttaaacggtgttggcattactcaacaggttctttcagagaaccaaaagcttattgccaataagtttaatcaggctctgggagctatgcaaacaggcttcactacaactaatgaagcttttcagaaggttcaggatgctgtgaacaacaatgcacaggctctatccaaattagctagcgagctatctaatacttttggtgctatttccgcctctattggagacatcatacaacgtcttgatgttctcgaacaggacgcccaaatagacagacttattaatggccgtttgacaacactaaatgcttttgttgcacagcagcttgttcgttccgaatcagctgctctttccgctcaattggctaaagataaagtcaatgagtgtgtcaaggcacaatccaagcgttctggattttgcggtcaaggcacacatatagtgtcctttgttgtaaatgcccctaatggcctttacttcatgcatgttggttattaccctagcaaccacattgaggttgtttctgcttatggtctttgcgatgcagctaaccctactaattgtatagcccctgttaatggctactttattaaaactaataacactaggattgttgatgagtggtcatatactggctcgtccttctatgcacctgagcccattacctcccttaatactaagtatgttgcaccacaggtgacataccaaaacatttctactaacctccctcctcctcttctcggcaattccaccgggattgacttccaagatgagttggatgagtttttcaaaaatgttagcaccagtatacctaattttggtttcctaacacagattaatactacattactcgatcttacctacgagatgttgtctcttcaacaagttgttaaagcccttaatgagtcttacatagaccttaaagagcttggcaattatacttattacaacaaatggccgtggtacatttggcttggtttcattgctgggcttgttgccttagctctatgcgtcttcttcatactgtgctgcactggttgtggcacaaactgtatgggaaaacttaagtgtaatcgttgttgtgatagatacgaggaatacgacctcgagccgcataaggttcatgttcactaattaacgaactattaatgagagttcaaagaccacccactctcttgttagtgttttcactctctcttttggtcactgcatcctcaaaacctctctatgtacctgagcattgtcagaattattctggttgcatgcttagggcttgtattaaaactgcccaagctgatacagctggtctttatacaaattttcgaattgacgtcccatctgcagaatcaactggtactcaatcagtttctgtcgatcttgagtcaacttcaactcatgatggtcctaccgaacatgttactagtgtgaatctttttgacgttggttactcagttaattaacgaactctatggattacgtgtctctgcttaatcaaatttggcagaagtaccttaactcaccgtatactacttgtttgtacatccctaaacccacagctaagtatacacctttagttggcacttcattgcaccctgtgctgtggaactgtcagctatcctttgctgggtatactgaatctgctgttaattctacaaaagctttggccaaacaggacgcagctcagcgaatcgcttggttgctacataaggatggaggaatccctgatggatgttccctctacctccggcactcaagtttattcgcgcaaagcgaggaagaggagccattctccaactaagaaactgcgctacgttaagcgtagattttctcttctgcgccatgaagaccttagtgttattgtccaaccaacacactatgtcagggttacattttcagaccccaacatgtggtatctacgttcgggtcatcatttacactcagttcacaattggcttaaaccttatggcggccaacctgtttctgagtaccatattactctagctttgctaaatctcactgatgaagatttagctagagatttttcacccattgcgctctttttgcgcaatgtcagatttgagctacatgagttcgccttgctgcgcaaaactcttgttcttaatgcatcagagatctactgtgctaacatacatagatttaagcctgtgtatagagttaacacggcaatccctactattaaggattggcttctcgttcagggattttccctttaccatagtggcctccctttacatatgtcaatctctaaattgcatgcactggatgatgttactcgcaattacatcattacaatgccatgctttagaacttaccctcaacaaatgtttgttactcctttggccgtagatgttgtctccatacggtcttccaatcagggtaataaacaaattgttcattcttatcccattttacatcatccaggattttaacgaactatggctttctcggcgtctttatttaaacccgtccagctagtcccagtttctcctgcatttcatcgcattgagtctactgactctattgttttcacatacattcctgctagcggctatgtagctgctttagctgtcaatgtgtgtctcattcccctattattactgctacgtcaagatacttgtcgtcgcagcattatcagaactatggttctctatttccttgttctgtataactttttattagccattgtactagtcaatggtgtacattatccaactggaagttgcctgatagccttcttagttatcctcataatactttggtttgtagatagaattcgtttctgtctcatgctgaattcctacattccactgtttgacatgcgttcccactttattcgtgttagtacagtttcttctcatggtatggtccctgtaatacacaccaaaccattatttattagaaacttcgatcagcgttgcagctgttctcgttgtttttatttgcactcttccacttatatagagtgcacttatattagccgttttagtaagattagcctagtttctgtaactgacttctccttaaacggcaatgtttccactgttttcgtgcctgcaacgcgcgattcagttcctcttcacataatcgccccgagctcgcttatcgtttaagcagctctgcgctactatgggtcccgtgtagaggctaatccattagtctctctttggacatatggaaaacgaactatgttaccctttgtccaagaacgaatagggttgttcatagtaaactttttcatttttaccgtagtatgtgctataacactcttggtgtgtatggctttccttacggctactagattatgtgtgcaatgtatgacaggcttcaataccctgttagttcagcccgcattatacttgtataatactggacgttcagtctatgtaaaattccaggatagtaaaccccctctaccacctgacgagtgggtttaacgaactccttcataatgtctaatatgatgcaactcactgaggcgcagattattgccattattaaagactggaactttgcatggtccctgatctttctcttaattactatcgtactacagtatggatacccatcccgtagtatgactgtctatgtctttaaaatgtttgttttatggctcctatggccatcttccatggcgctatcaatatttagcgccgtttatccaattgatctagcttcccagataatctctggcattgtagcagctgtttcagctatgatgtggatttcctactttgtgcagagtatccggctgtttatgagaactggatcatggtggtcattcaatcctgagactaattgccttttgaacgttccatttggtggtacaactgtcgtacgtccactcgtagaggactctaccagtgtaactgctgttgtaaccaatggccacctcaaaatggctggcatgcatttcggtgcttgtgactacgacagacttcctaatgaagtcaccgtggccaaacccaatgtgctgattgctttaaaaatggtgaagcggcaaagctacggaactaattccggcgttgccatttaccatagatataaggcaggtaattacaggagtccgcctattacggcggatattgaacttgcattgcttcgagcttaggctctttagtaagagtatcttaattgattttaacgaatctcaatttcattgttatggcatcccctgctgcacctcgtgctgtttcctttgccgataacaatgatataacaaatacaaacctatctcgaggtagaggacgtaatccaaaaccacgagctgcaccaaataacactgtctcttggtacactgggcttacccaacacgggaaagtccctcttacctttccacctgggcagggtgtacctcttaatgccaattctacccctgcgcaaaatgctgggtattggcggagacaggacagaaaaattaataccgggaatggaattaagcaactggctcccaggtggtacttctactacactggaactggacccgaagcagcactcccattccgggctgttaaggatggcatcgtttgggtccatgaagatggcgccactgatgctccttcaacttttgggacgcggaaccctaacaatgattcagctattgttacacaattcgcgcccggtactaagcttcctaaaaacttccacattgaggggactggaggcaatagtcaatcatcttcaagagcctctagcttaagcagaaactcttccagatctagttcacaaggttcaagatcaggaaactctacccgcggcacttctccaggtccatctggaatcggagcagtaggaggtgatctactttaccttgatcttctgaacagactacaagcccttgagtctggcaaagtaaagcaatcgcagccaaaagtaatcactaagaaagatgctgctgctgctaaaaataagatgcgccacaagcgcacttccaccaaaagtttcaacatggtgcaagcttttggtcttcgcggaccaggagacctccagggaaactttggtgatcttcaattgaataaactcggcactgaggacccacgttggccccaaattgctgagcttgctcctacagccagtgcttttatgggtatgtcgcaatttaaacttacccatcagaacaatgatgatcatggcaaccctgtgtacttccttcggtacagtggagccattaaacttgacccaaagaatcccaactacaataagtggttggagcttcttgagcaaaatattgatgcctacaaaaccttccctaagaaggaaaagaaacaaaaggcaccaaaagaagaatcaacagaccaaatgtctgaacctccaaaggagcagcgtgtgcaaggtagcatcactcagcgcactcgcacccgtccaagtgttcagcctggtccaatgattgatgttaacactgattagtgtcactcaaagtaacaagatcgcggcaatcgtttgtgtttggcaaccccatctcaccatcgcttgtccactcttgcacagaatggaatcatgttgtaattacagtgcaataaggtaattataacccatttaattgatagctatgctttattaaagtgtgtagctgtagagagaatgttaaagactgtcacctctgcttgattgcaagtgaacagtgccccccgggaagagctctacagtgtgaaatgtaaataaaaaatagctattattcaattagattaggctaattagatgatttgcaaaaaaaaaaaa

SEQ ID NO: 2 - RNA, 30119 nt, unidentified
  source 1..30119: mol_type = other RNA
gatttaagtgaatagcttggctatctcacttcccctcgttctcttgcagaactttgattttaacgaacttaaataaaagccctgttgtttagcgtatcgttgcacttgtctggtgggattgtggcattaatttgcctgctcatctaggcagtggacatatgctcaacactgggtataattctaattgaatactatttttcagttagagcgtcgtgtctcttgtacgtctcggtcacaatacacggtttcgtccggtgcgtggcaattcggggcacatcatgtctttcgtggctggtgtgaccgcgcaaggtgcgcgcggtacgtatcgagcagcgctcaactctgaaaaacatcaagaccatgtgtctctaactgtgccactctgtggttcaggaaacctggttgaaaaactttcaccatggttcatggatggcgaaaatgcctatgaagtggtgaaggccatgttacttaaaaaggagccacttctctatgtgcccatccggctggctggacacactagacacctcccaggtcctcgtgtgtacctggttgagaggctcattgcttgtgaaaatccattcatggttaaccaattggcttatagctctagtgcaaatggcagcctggttggcacaactttgcagggcaagcctattggtatgttcttcccttatgacatcgaacttgtcacaggaaagcaaaatattctcctgcgcaagtatggccgtggtggttatcactacaccccattccactatgagcgagacaacacctcttgccctgagtggatggacgattttgaggcggatcctaaaggcaaatatgcccagaatctgcttaagaagttgattggcggtgatgtcactccagttgaccaatacatgtgtggcgttgatggaaaacccattagtgcctacgcatttttaatggccaaggatggaataaccaaactggctgatgttgaagcggacgtcgcagcacgtgctgatgacgaaggcttcatcacattaaagaacaatctatatagattggtttggcatgttgagcgtaaagacgttccatatcctaagcaatctatttttactattaatagtgtggtccaaaaggatggtgttgaaaacactcctcctcactattttactcttggatgcaaaattttaacgctcaccccacgcaacaagtggagtggcgtttctgacttgtccctcaaacaaaaactcctttacaccttctatggtaaggagtcacttgagaacccaacctacatttaccactccgcattcattgagtgtggaagttgtggtaatgattcctggcttacagggaatgctatccaagggtttgcctgtggatgtggggcatcatatacagctaatgatgtcgaagtccaatcatctggcatgattaagccaaatgctcttctttgtgctacttgcccctttgctaagggtgatagctgttcttctaattgcaaacattcagttgctcagttggttagttacctttctgaacgctgtaatgttattgctgattctaagtccttcacacttatctttggtggcgtagcttacgcctactttggatgtgaggaaggtactatgtactttgtgcctagagctaagtctgttgtctcaaggattggagactccatctttacaggctgtactggctcttggaacaaggtcactcaaattgctaacatgttcttggaacagactcagcattcccttaactttgtgggagagttcgttgtcaacgatgttgtcctcgcaattctctctggaaccacaactaatgttgacaaaatacgccagcttctcaaaggtgtcacccttgacaagttgcgtgattatttagctgactatgacgtagcagtcactgccggcccattcatggataatgctattaatgttggtggtacaggattacagtatgccgccattactgcaccttatgtagttctcactggcttaggtgagtcctttaagaaagttgcaaccataccgtataaggtttgcaactctgttaaggatactctggcttattatgctcacagcgtgttgtacagagtttttccttatgacatggattctggtgtgtcatcctttagtgaactactttttgattgcgttgatctttcagtagcttctacctattttttagtccgcatcttgcaagataagactggcgactttatgtctacaattattacttcctgccaaactgctgttagtaagcttctagatacatgttttgaagctacagaagcaacatttaacttcttgttagatttggcaggattgttcagaatctttctccgcaatgcctatgtgtacacttcacaagggtttgtggtggtcaatggcaaagtttctacacttgtcaaacaagtgttagacttgcttaataagggtatgcaacttttgcatacaaaggtctcctgggctggttctaaaatcattgctgttatctacagcggcagggagtctctaatattcccatcgggaacctattactgtgtcaccactaaggctaagtccgttcaacaagatcttgacgttattttgcctggtgagttttccaagaagcagttaggactgctccaacctactgacaattctacaactgttagtgttactgtatccagtaacatggttgaaactgttgtgggtcaacttgagcaaactaatatgcatagtcctgatgttatagtaggtgactatgtcattattagtgaaaaattgtttgtgcgtagtaaggaagaagacggatttgccttctaccctgcttgcactaatggtcatgctgtaccgactctctttagacttaagggaggtgcacctgtaaaaaaagtagcctttggcggtgatcaagtacatgaggttgctgctgtaagaagtgttactgtcgagtacaacattcatgctgtattagacacactacttgcttcttctagtcttagaacctttgttgtagataagtctttgtcaattgaggagtttgctgacgtagtaaaggaacaagtctcagacttgcttgttaaattactgcgtggaatgccgattccagattttgatttagacgattttattgacgcaccatgctattgctttaacgctgagggtgatgcatcctggtcttctactatgatcttctctcttcaccccgtcgagtgtgacgaggagtgttctgaagtagaggcttcagatttagaagaaggtgaatcagagtgcatttctgagacttcaactgaacaagttgacgtttctcatgagacttctgacgacgagtgggctgctgcagttgatgaagcgttccctctcgatgaagcagaagatgttactgaatctgtgcaagaagaagcacaaccagtagaagtacctgttgaagatattgcgcaggttgtcatagctgacaccttacaggaaactcctgttgtgcctgatactgttgaagtcccaccgcaagtggtgaaacttccgtctgcacctcagactatccagcccgaggtaaaagaagttgcacctgtctatgaggctgataccgaacagacacagaatgttactgttaaacctaagaggttacgcaaaaagcgtaatgttgaccctttgtccaattttgaacataaggttattacagagtgcgttaccatagttttaggtgacgcaattcaagtagccaagtgctatggggagtctgtgttagttaatgctgctaacacacatcttaagcatggcggtggtatcgctggtgctattaatgcggcttcaaaaggggctgtccaaaaagagtcagatgagtatattctggctaaagggccgttacaagtaggagattcagttctcttgcaaggccattctctagctaagaatatcctgcatgtcgtaggcccagatgcccgcgctaaacaggatgtttctctccttagtaagtgctataaggctatgaatgcatatcctcttgtagtcactcctcttgtttcagcaggcatatttggtgtaaaaccagctgtgtcttttgattatcttattagggaggctaagactagagttttagtcgtcgttaattcccaagatgtctataagagtcttaccatagttgacattccacagagtttgactttttcatatgatgggttacgtggcgcaatacgtaaagctaaagattatggttttactgtttttgtgtgcacagacaactctgctaacactaaagttcttaggaacaagggtgttgattatactaagaagtttcttacagttgacggtgtgcaatattattgctacacgtctaaggacactttagatgatatcttacaacaggctaataagtctgttggtattatatctatgcctttgggatatgtgtctcatggtttagacttaatgcaagcagggagtgtcgtgcgtagagttaacgtgccctacgtgtgtctcctagctaataaagagcaagaagctattttgatgtctgaagacgttaagttaaacccttcagaagattttataaagcacgtccgcactaatggtggttacaattcttggcatttagtcgagggtgaactattggtgcaagacttacgcttaaataagctcctgcattggtctgatcaaaccatatgctacaaggatagtgtgttttatgttgtaaagaatagtacagcttttccatttgaaacactttcagcatgtcgtgcgtatttggattcacgcacgacacagcagttaacaatcgaagtcttagtgactgtcgatggtgtaaattttagaacagtcgttctaaataataagaacacttatagatcacagcttggatgcgttttctttaatggtgctgatatttctgacaccattcctgatgagaaacagaatggtcacagtttatatctagcagacaatttgactgctgatgaaacaaaggcgcttaaagagttatatggccccgttgatcctactttcttacacagattctattcacttaaggctgcagtccatgggtggaagatggttgtgtgtgataaggtacgttctctcaaattgagtgataataattgttatcttaatgcagttattatgacacttgatttattgaaggacattaaatttgttatacctgctctacagcatgcatttatgaaacataagggcggtgattcaactgacttcatagccctcattatggcttatggcaattgcacatttggtgctccagatgatgcctctcggttacttcataccgtgcttgcaaaggctgagttatgctgttctgcacgcatggtttggagagagtggtgcaatgtctgtggcataaaagatgttgttctacaaggcttaaaagcttgttgttacgtgggtgtgcaaactgttgaagatctgcgtgctcgcatgacatatgtatgccagtgtggtggtgaacgtcatcggcaattagtcgaacacaccaccccctggttgctgctctcaggcacaccaaatgaaaaattggtgacaacctccacggcgcctgattttgtagcatttaatgtctttcagggcattgaaacggctgttggccattatgttcatgctcgcctgaagggtggtcttattttaaagtttgactctggcaccgttagcaagacttcagactggaagtgcaaggtgacagatgtacttttccccggccaaaaatacagtagcgattgtaatgtcgtacggtattctttggacggtaatttcagaacagaggttgatcccgacctatctgctttctatgttaaggatggtaaatactttacaagtgaaccacccgtaacatattcaccagctacaattttagctggtagtgtctacactaatagctgccttgtatcgtctgatggacaacctggcggtgatgctattagtttgagttttaataaccttttagggtttgattctagtaaaccagtcactaagaaatacacttactccttcttgcctaaagaagacggcgatgtgttgttggctgagtttgacacttatgaccctatttataagaatggtgccatgtataaaggcaaaccaattctttgggtcaataaagcatcttatgatactaatcttaataagttcaatagagctagtttgcgtcaaatttttgacgtagcccccattgaactcgaaaataaattcacacctttgagtgtggagtctacaccagttgaacctccaactgtagatgtggtagcacttcaacaggaaatgacaattgtcaaatgtaagggtttaaataaacctttcgtgaaggacaatgtcagtttcgttgctgatgattcaggtactcccgttgttgagtatctgtctaaagaagacctacatacattgtatgtagaccctaagtatcaagtcattgtcttaaaagacaatgtactttcttctatgcttagattgcacaccgttgagtcaggtgatattaacgttgttgcagcttccggatctttgacacgtaaagtgaagttactatttagggcttcattttatttcaaagaatttgctacccgcactttcactgctaccactgctgtaggtagttgtataaagagtgtagtgcggcatctaggtgttactaaaggcatattgacaggctgttttagttttgccaagatgttatttatgcttccactagcttactttagtgattcaaaactcggcaccacagaggttaaagtgagtgctttgaaaacagccggcgttgtgacaggtaatgttgtaaaacagtgttgcactgctgctgttgatttaagtatggataagttgcgccgtgtggattggaaatcaaccctacggttgttacttatgttatgcacaactatggtattgttgtcttctgtgtatcacttgtatgtcttcaatcaggtcttatcaagtgatgttatgtttgaagatgcccaaggtttgaaaaagttctacaaagaagttagagcttacctaggaatctcttctgcttgtgacggtcttgcttcagcttatagggcgaattcctttgatgtacctacattctgcgcaaaccgttctgcaatgtgtaattggtgcttgattagccaagattccataactcactacccagctcttaagatggttcaaacacatcttagccactatgttcttaacatagattggttgtggtttgcatttgagactggtttggcatacatgctctatacctcggccttcaactggttgttgttggcaggtacattgcattatttctttgcacagacttccatatttgtagactggcggtcatacaattatgctgtgtctagtgccttctggttattcacccacattccaatggcgggtttggtacgaatgtataatttgttagcatgcctttggcttttacgcaagttttatcagcatgtaatcaatggttgcaaagatacggcatgcttgctctgctataagaggaaccgacttactagagttgaagcttctaccgttgtctgtggtggaaaacgtacgttttatatcacagcaaatggcggtatttcattctgtcgtaggcataattggaattgtgtggattgtgacactgcaggtgtggggaataccttcatctgtgaagaagtcgcaaatgacctcactaccgccctacgcaggcctattaacgctacggatagatcacattattatgtggattccgttacagttaaagagactgttgttcagtttaattatcgtagagacggtcaaccattctacgagcggtttcccctctgcgcttttacaaatctagataagttgaagttcaaagaggtctgtaaaactactactggtatacctgaatacaactttatcatctacgactcatcagatcgtggccaggaaagtttagctaggtctgcatgtgtttattattctcaagtcttgtgtaaatcaattcttttggttgactcaagtttggttacttctgttggtgattctagtgaaatcgccactaaaatgtttgattcctttgttaatagtttcgtctcgctgtataatgtcacacgcgataagttggaaaaacttatctctactgctcgtgatggcgtaaggcgaggcgataacttccatagtgtcttaacaacattcattgacgcagcacgaggccccgcaggtgtggagtctgatgttgagaccaatgaaattgttgactctgtgcagtatgctcataaacatgacatacaaattactaatgagagctacaataattatgtaccctcatatgttaaacctgatagtgtgtctaccagcgatttaggtagtctcattgattgtaatgcggcttcagttaaccaaattgtcttgcgtaattctaatggtgcttgcatttggaacgctgctgcatatatgaaactctcggatgcacttaaacgacagattcgcattgcatgccgtaagtgtaatttagctttccggttaaccacctcaaagctacgcgctaatgataatatcttatcagttagattcactgctaacaaaattgttggtggtgctcctacatggtttaatgcgttgcgtgactttacgttaaagggttatgttcttgctaccattattgtgtttctgtgtgctgtactgatgtatttgtgtttacctacattttctatggcacctgttgaattttatgaagaccgcatcttggactttaaagttcttgataatggtatcattagggatgtaaatcctgatgataagtgctttgctaataagcaccggtccttcacacaatggtatcatgagcatgttggtggtgtctatgacaactctatcacatgcccattgacagttgcagtaattgctggagttgctggtgctcgcattccagacgtacctactacattggcttgggtgaacaatcagataattttctttgtttctcgagtctttgctaatacaggcagtgtttgctacactcctatagatgagataccctataagagtttctctgatagtggttgcattcttccatctgagtgcactatgtttagggatgcagagggccgtatgacaccatactgccatgatcctactgttttgcctggggcttttgcgtacagtcagatgaggcctcatgttcgttacgacttgtatgatggtaacatgtttattaaatttcctgaagtagtatttgaaagtacacttaggattactagaactctgtcaactcagtactgccggttcggtagttgtgagtatgcacaagagggtgtttgtattaccacaaatggctcgtgggccatttttaatgaccaccatcttaatagacctggtgtctattgtggctctgattttattgacattgtcaggcggttagcagtatcactgttccagcctattacttatttccaattgactacctcattggtcttgggtataggtttgtgtgcgttcctgactttgctcttctattatattaataaagtaaaacgtgcttttgcagattacacccagtgtgctgtaattgctgttgttgctgctgttcttaatagcttgtgcatctgctttgttacctctataccattgtgtatagtaccttacactgcattgtactattatgctacattctattttactaatgagcctgcatttattatgcatgtttcttggtacattatgttcgggcctatcgttcccatatggatgacctgcgtctatacagttgcaatgtgctttagacacttcttctgggttttagcttattttagtaagaaacatgtagaagtttttactgatggtaagcttaattgtagtttccaggacgctgcctctaatatctttgttattaacaaggacacttatgcagctcttagaaactctttaactaatgatgcctattcacgatttttggggttgtttaacaagtataagtacttctctggtgctatggaaacagccgcttatcgtgaagctgcagcatgtcatcttgctaaagccttacaaacatacagcgagactggtagtgatcttctttaccaaccacccaactgtagcataacctctggcgtgttgcaaagcggtttggtgaaaatgtcacatcccagtggagatgttgaggcttgtatggttcaggttacctgcggtagcatgactcttaatggtctttggcttgacaacacagtctggtgcccacgacacgtaatgtgcccggctgaccagttgtctgatcctaattatgatgccttgttgatttctatgactaatcatagtttcagtgtgcaaaaacacattggcgctccagcaaacttgcgtgttgttggtcatgccatgcaaggcactcttttgaagttgactgtcgatgttgctaaccctagcactccagcctacacttttacaacagtgaaacctggcgcagcatttagtgtgttagcatgctataatggtcgtccgactggtacattcactgttgtaatgcgccctaactacacaattaagggttcctttctgtgtggttcttgtggtagtgttggttacaccaaggagggtagtgtgatcaatttctgttacatgcatcaaatggaacttgctaatggtacacataccggttcagcatttgatggtactatgtatggtgcctttatggataaacaagtgcaccaagttcagttaacagacaaatactgcagtgttaatgtagtagcttggctttacgcagcaatacttaatggttgcgcttggtttgtaaaacctaatcgcactagtgttgtttcttttaatgaatgggctcttgccaaccaattcactgaatttgttggcactcaatccgttgacatgttagctgtcaaaacaggcgttgctattgaacagctgctttatgcgatccaacaactgtatactgggttccagggaaagcaaatccttggcagtaccatgttggaagatgaattcacacctgaggatgttaatatgcagattatgggtgtggttatgcagagtggtgtgagaaaagttacatatggtactgcgcattggttgtttgcgacccttgtctcaacctatgtgataatcttacaagccactaaatttactttgtggaactacttgtttgagactattcccacacagttgttcccactcttatttgtgactatggccttcgttatgttgttggttaaacacaaacacacctttttgacacttttcttgttgcctgtggctatttgtttgacttatgcaaacatagtctacgagcccactactcccatttcgtcagcgctgattgcagttgcaaattggcttgcccccactaatgcttatatgcgcactacacatactgatattggtgtctacattagtatgtcacttgtattagtcattgtagtgaagagattgtacaacccatcactttctaactttgcgttagcattgtgcagtggtgtaatgtggttgtacacttatagcattggagaagcctcaagccccattgcctatctggtttttgtcactacactcactagtgattatacgattacagtctttgttactgtcaaccttgcaaaagtttgcacttatgccatctttgcttactcaccacagcttacacttgtgtttccggaagtgaagatgatacttttattatacacatgtttaggtttcatgtgtacttgctattttggtgtcttctctcttttgaaccttaagcttagagcacctatgggtgtctatgactttaaggtctcaacacaagagttcagattcatgactgctaacaatctaactgcacctagaaattcttgggaggctatggctctgaactttaagttaataggtattggcggtacaccttgtataaaggttgctgctatgcagtctaaacttacagatcttaaatgcacatctgtggttctcctctctgtgctccaacagttacacttagaggctaatagtagggcctgggctttctgtgttaaatgccataatgatatattggcagcaacagaccccagtgaggctttcgagaaattcgtaagtctctttgctactttaatgactttttctggtaatgtagatcttgatgcgttagctagtgatatttttgacactcctagcgtacttcaagctactctttctgagttttcacacttagctacctttgctgagttggaagctgcgcagaaagcctatcaggaagctatggactctggtgacacctcaccacaagttcttaaggctttgcagaaggctgttaatatagctaaaaacgcctatgagaaggataaggcagtggcccgtaagttagaacgtatggctgatcaggctatgacttctatgtataagcaagcacgtgctgaagacaagaaagcaaaaattgtcagtgctatgcaaactatgttgtttggtatgattaagaagctcgacaacgatgttcttaatggtatcatttctaacgctaggaatggttgtatacctcttagtgtcatcccactgtgtgcttcaaataaacttcgcgttgtaattcctgacttcaccgtctggaatcaggtagtcacatatccctcgcttaactacgctggggctttgtgggacattacagttataaacaatgtggacaatgaaattgttaagtcttcagatgttgtagacagcaatgaaaatttaacatggccacttgttttagaatgcactagggcatccacttctgccgttaagttgcaaaataatgagatcaaaccttcaggtctaaaaaccatggttgtgtctgcgggtcaagagcaaactaactgtaatactagttccttagcttattacgaacctgtgcagggtcgtaaaatgctgatggctcttctttctgataatgcctatctcaaatgggcgcgtgttgaaggtaaggacggatttgtcagtgtagagctacaacctccttgcaaattcttgattgcgggaccaaaaggacctgaaatccgatatctctattttgttaaaaatcttaacaaccttcatcgcgggcaagtgttagggcacattgctgcgactgttagattgcaagctggttctaacaccgagtttgcctctaattcctcggtgttgtcacttgttaacttcaccgttgatcctcaaaaagcttatctcgatttcgtcaatgcgggaggtgccccattgacaaattgtgttaagatgcttactcctaaaactggtacaggtatagctatatctgttaaaccagagagtacagctgatcaagagacttatggtggagcttcagtgtgtctctattgccgtgcgcatatagaacatcctgatgtctctggtgtttgtaaatataagggtaagtttgtccaaatccctgctcagtgtgtccgtgaccctgtgggattttgtttgtcaaataccccctgtaatgtctgtcaatattggattggatatgggtgcaattgtgactcgcttaggcaagcagcactgccccaatctaaagattccaattttttaaacgagtccggggttctattgtaaatgcccgaatagaaccctgttcaagtggtttgtccactgatgtcgtctttagggcatttgacatctgcaactataaggctaaggttgctggtattggaaaatactacaagactaatacttgtaggtttgtagaattagatgaccaagggcatcatttagactcctattttgtcgttaagaggcatactatggagaattatgaactagagaagcactgttacgacttgttacgtgactgtgatgctgtagctccccatgatttcttcatctttgatgtagacaaagttaaaacacctcatattgtacgtcagcgtttaactgagtacactatgatggatcttgtatatgccctgaggcactttgatcaaaatagcgaagtgcttaaggctatcttagtgaagtatggttgctgtgatgttacctactttgaaaataaactctggtttgattttgttgaaaatcccagtgttattggtgtttatcataaacttggagaacgtgtacgccaagctatcttaaacactgttaaattttgtgaccacatggtcaaggctggtttagtcggtgtgctcacactagacaaccaggaccttaatggcaagtggtatgattttggtgacttcgtaatcactcaacctggttcaggagtagctatagttgatagctactattcttatttgatgcctgtgctctcaatgaccgattgtctggccgctgagacacatagggattgtgattttaataaaccactcattgagtggccacttactgagtatgattttactgattataaggtacaactctttgagaagtactttaaatattgggatcagacgtatcacgcaaattgcgttaattgtactgatgaccgttgtgtgttacattgtgctaatttcaatgtattgtttgctatgaccatgcctaagacttgtttcggacccatagtccgaaagatctttgttgatggcgtgccatttgtagtatcttgtggttatcactacaaagaattaggtttagtcatgaatatggatgttagtctccatagacataggctctctcttaaggagttgatgatgtatgccgctgatccagccatgcacattgcctcctctaacgcttttcttgatttgaggacatcatgttttagtgtcgctgcacttacaactggtttgacttttcaaactgtgcggcctggcaattttaaccaagacttctatgatttcgtggtatctaaaggtttctttaaggagggctcttcagtgacgctcaaacattttttctttgctcaagatggtaatgctgctattacagattataattactattcttataatctgcctactatgtgtgacatcaaacaaatgttgttctgcatggaagttgtaaacaagtacttcgaaatctatgacggtggttgtcttaatgcttctgaagtggttgttaataatttagacaagagtgctggccatccttttaataagtttggcaaagctcgtgtctattatgagagcatgtcttaccaggagcaagatgaactttttgccatgacaaagcgtaacgtcattcctaccatgactcaaatgaatctaaaatatgctattagtgctaagaatagagctcgcactgttgcaggcgtgtccatacttagcacaatgactaatcgccagtaccatcagaaaatgcttaagtccatggctgcaactcgtggagcgacttgcgtcattggtactacaaagttctacggtggctgggatttcatgcttaaaacattgtacaaagatgttgataatccgcatcttatgggttgggattaccctaagtgtgatagagctatgcctaatatgtgtagaatcttcgcttcactcatattagctcgtaaacatggcacttgttgtactacaagggacagattttatcgcttggcaaatgagtgtgctcaggtgctaagcgaatatgttctatgtggtggtggttactacgtcaaacctggaggtaccagtagcggagatgccaccactgcatatgccaatagtgtctttaacattttgcaggcgacaactgctaatgtcagtgcacttatgggtgctaatggcaacaagattgttgacaaagaagttaaagacatgcagtttgatttgtatgtcaatgtttacaggagcactagcccagaccccaaatttgttgataaatactatgcttttcttaataagcacttttctatgatgatactgtctgatgacggtgtcgtttgctataatagtgattatgcagctaagggttacattgctggaatacagaattttaaggaaacgctgtattatcagaacaatgtctttatgtctgaagctaaatgctgggtggaaaccgatctgaagaaagggccacatgaattctgttcacagcatacgctttatattaaggatggcgacgatggttacttccttccttatccagacccttcaagaattttgtctgccggttgctttgtagatgatatcgttaagactgacggtacactcatggtagagcggtttgtgtctttggctatagatgcttaccctctcacaaagcatgaagatatagaataccagaatgtattctgggtctacttacagtatatagaaaaactgtataaagaccttacaggacacatgcttgacagttattctgtcatgctatgtggtgataattctgctaagttttgggaagaggcattctatagagatctctatagttcgcctaccactttgcaggctgtcggttcatgcgttgtatgccattcacagacttccctacgctgtgggacatgcatccgtagaccatttctctgctgtaaatgctgctatgatcatgttatagcaactccacataagatggttttgtctgtttctccttacgtttgtaatgcccctggttgtggcgtttcagacgttactaagctatatttaggtggtatgagctacttttgtgtagatcatagacctgtgtgtagttttccactttgcgctaatggtcttgtattcggcttatacaagaatatgtgcacaggtagtccttctatagttgaatttaataggttggctacctgtgactggactgaaagtggtgattacacccttgccaatactacaacagaaccactcaaactttttgctgctgagactttacgtgccactgaagaggcgtctaagcagtcttatgctattgccaccatcaaagaaattgttggtgagcgccaactattacttgtgtgggaggctggcaagtccaaaccaccactcaatcgtaattatgtttttactggttatcatataaccaaaaatagtaaagtgcagctcggtgagtacattttcgagcgcattgattatagtgatgctgtatcctacaagtctagtacaacgtataaactgactgtaggtgacatcttcgtacttacctctcactctgtggctaccttgacggcgcccacaattgtgaatcaagagaggtatgttaaaattactgggttgtacccaaccattacggtacctgaagagttcgcaagtcatgttgccaacttccaaaaatcaggttatagtaaatatgtcactgttcagggaccacctggcactggcaaaagtcattttgctatagggttagcgatttactaccctacagcacgtgttgtttatacagcatgttcacacgcagctgttgatgctttgtgtgaaaaagcttttaaatatttgaacattgctaaatgttcccgtatcattcctgcaaaggcacgtgttgagtgctatgacaggtttaaagttaatgagacaaattctcaatatttgtttagtactattaatgctctaccagaaacttctgccgatattctggtggttgatgaggttagtatgtgcactaattatgatctttcaattattaatgcacgtattaaagctaagcacattgtctatgtaggagatccagcacagttgccagctcctaggactttgttgactagaggcacattggaaccagaaaatttcaatagtgtcactagattgatgtgtaacttaggtcctgacatatttttaagtatgtgctacaggtgtcctaaggaaatagtaagcactgtgagcgctcttgtctacaataataaattgttagccaagaaggagctttcaggccagtgctttaaaatactctataagggcaatgtgacgcatgatgctagctctgccattaatagaccacaactcacatttgtgaagaattttattactgccaatccggcatggagtaaggcagtctttatttcgccttacaattcacagaatgctgtgtctcgttcaatgctgggtcttaccactcagactgttgattcctcacagggttcagaataccagtacgttatcttctgtcaaacagcagatacggcacatgctaacaacattaacagatttaatgttgcaatcactcgtgcccaaaaaggtattctttgtgttatgacatctcaggcactctttgagtccttagagtttactgaattgtcttttactaattacaagctccagtctcagattgtaactggcctttttaaagattgctctagagaaacttctggcctctcacctgcttatgcaccaacatatgttagtgttgatgacaagtataagacgagtgatgagctttgcgtgaatcttaatttacccgcaaatgtcccatactctcgtgttatttccaggatgggctttaaactcgatgcaacagttcctggatatcctaagcttttcattactcgtgaagaggctgtaaggcaagttcgaagctggataggcttcgatgttgagggtgctcatgcttcccgtaatgcatgtggcaccaatgtgcctctacaattaggattttcaactggtgtgaactttgttgttcagccagttggtgttgtagacactgagtggggtaacatgttaacgggcattgctgcacgtcctccaccaggtgaacagtttaagcacctcgtgcctcttatgcataagggggctgcgtggcctattgttagacgacgtatagtgcaaatgttgtcagacactttagacaaattgtctgattactgtacgtttgtttgttgggctcatggctttgaattaacgtctgcatcatacttttgcaagataggtaaggaacagaagtgttgcatgtgcaatagacgcgctgcagcgtactcttcacctctgcaatcttatgcctgctggactcattcctgcggttatgattatgtctacaaccctttctttgtcgatgttcaacagtggggttatgtaggcaatcttgctactaatcacgatcgttattgctctgtccatcaaggagctcatgtggcttctaatgatgcaataatgactcgttgtttagctattcattcttgttttatagaacgtgtggattgggatatagagtatccttatatctcacatgaaaagaaattgaattcctgttgtagaatcgttgagcgcaacgtcgtacgtgctgctcttcttgccggttcatttgacaaagtctatgatattggcaatcctaaaggaattcctattgttgatgaccctgtggttgattggcattattttgatgcacagcccttgaccaggaaggtacaacagcttttctatacagaggacatggcctcaagatttgctgatgggctctgcttattttggaactgtaatgtaccaaaatatcctaataatgcaattgtatgcaggtttgacacacgtgtgcattctgagttcaatttgccaggttgtgatggcggtagtttgtatgttaacaagcacgcttttcatacaccagcatatgatgtgagtgcattccgtgatctgaaacctttaccattcttttattattctactacaccatgtgaagtgcatggtaatggtagtatgatagaggatattgattatgtacccctaaaatctgcagtctgtattacagcttgtaatttagggggcgctgtttgtaggaagcatgctacagagtacagagagtatatggaagcatataatcttgtctctgcatcaggtttccgcctttggtgttataagacctttgatatttataatctctggtctacttttacaaaagttcaaggtttggaaaacattgcttttaatgttgttaaacaaggccattttattggtgttgagggtgaactacctgtagctgtagtcaatgataagatcttcaccaagagtggcgttaatgacatttgtatgtttgagaataaaaccactttgcctactaatatagcttttgaactctatgctaagcgtgctgtacgctcgcatcccgatttcaaattgctacacaatttacaagcagacatttgctacaagttcgtcctttgggattatgaacgtagcaatatttatggtactgctactattggtgtatgtaagtacactgatattgatgttaattcagctttgaatatatgttttgacatacgcgataattgttcattggagaagttcatgtctactcccaatgccatctttatttctgatagaaaaatcaagaaatacccttgtatggtaggtcctgattatgcttacttcaatggtgctatcatccgtgatagtgatgttgttaaacaaccagtgaagttctacttgtataagaaagtcaataatgagtttattgatcctactgagtgtatttacactcagagtcgctcttgtagtgacttcctacccctttctgacatggagaaagactttctatcttttgatagtgatgttttcattaagaagtatggcttggaaaactatgcttttgagcacgtagtctatggagacttctctcatactacgttaggcggtcttcacttgcttattggtttatacaagaagcaacaggaaggtcatattattatggaagaaatgctaaaaggtagctcaactattcataactattttattactgagactaacacagcggcttttaaggcggtgtgttctgttatagatttaaagcttgacgactttgttatgattttaaagagtcaagaccttggcgtagtatccaaggttgtcaaggttcctattgacttaacaatgattgagtttatgttatggtgtaaggatggacaggttcaaaccttctaccctcgactccaggcttctgcagattggaaacctggtcatgcaatgccatccctctttaaagttcaaaatgtaaaccttgaacgttgtgagcttgctaattacaagcaatctattcctatgcctcgcggtgtgcacatgaacatcgctaaatatatgcaattgtgccagtatttaaatacttgcacattagccgtgcctgccaatatgcgtgttatacattttggcgctggttctgataaaggtatcgctcctggtacctcagttttacgacagtggcttcctacagatgccattattatagataatgatttaaatgagttcgtgtcagatgctgacataactttatttggagattgtgtaactgtacgtgtcggccaacaagtggatcttgttatttccgacatgtatgatcctactactaagaatgtaacaggtagtaatgagtcaaaggctttattctttacttacctgtgtaacctcattaataataatcttgctcttggtgggtctgttgctattaaaataacagaacactcttggagcgttgaactttatgaacttatgggaaaatttgcttggtggactgttttctgcaccaatgcaaatgcatcctcatctgaaggattcctcttaggtattaattacttgggtactattaaagaaaatatagatggtggtgctatgcacgccaactatatattttggagaaattccactcctatgaatctgagtacttactcactttttgatttatccaagtttcaattaaaattaaaaggaacaccagttcttcaattaaaggagagtcaaattaacgaactcgtaatatctctcctgtcgcagggtaagttacttatccgtgacaatgatacactcagtgtttctactgatgttcttgttaacacctacagaaagttacgttgatgtagggccagattctgttaagtctgcttgtattgaggttgatatacaacagactttctttgataaaacttggcctaggccaattgatgtttctaaggctgacggtattatataccctcaaggccgtacatattctaacataactatcacttatcaaggtctttttccctatcagggagaccatggtgatatgtatgtttactctgcaggacatgctacaggcacaactccacaaaagttgtttgtagctaactattctcaggacgtcaaacagtttgctaatgggtttgtcgtccgtataggagcagctgccaattccactggcactgttattattagcccatctaccagcgctactatacgaaaaatttaccctgcttttatgctgggttcttcagttggtaatttctcagatggtaaaatgggccgcttcttcaatcatactctagttcttttgcccgatggatgtggcactttacttagagctttttattgtattctagagcctcgctctggaaatcattgtcctgctggcaattcctatacttcttttgccacttatcacactcctgcaacagattgttctgatggcaattacaatcgtaatgccagtctgaactcttttaaggagtattttaatttacgtaactgcacctttatgtacacttataacattaccgaagatgagattttagagtggtttggcattacacaaactgctcaaggtgttcacctcttctcatctcggtatgttgatttgtacggcggcaatatgtttcaatttgccaccttgcctgtttatgatactattaagtattattctatcattcctcacagtattcgttctatccaaagtgatagaaaagcttgggctgccttctacgtatataaacttcaaccgttaactttcctgttggatttttctgttgatggttatatacgcagagctatagactgtggttttaatgatttgtcacaactccactgctcatatgaatccttcgatgttgaatctggagtttattcagtttcgtctttcgaagcaaaaccttctggctcagttgtggaacaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcagcaattgctagcaactgttattcttcactgattttggattacttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaacaagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagcttgaatttgctaatgacacaaaaattgcctctcaattaggcaattgcgtggaatattccctctatggtgtttcgggccgtggtgtttttcagaattgcacagctgtaggtgttcgacagcagcgctttgtttatgatgcgtaccagaatttagttggctattattctgatgatggcaactactactgtttgcgtgcttgtgttagtgttcctgtttctgtcatctatgataaagaaactaaaacccacgctactctatttggtagtgttgcatgtgaacacatttcttctaccatgtctcaatactcccgttctacgcgatcaatgcttaaacggcgagattctacatatggcccccttcagacacctgttggttgtgtcctaggacttgttaattcctctttgttcgtagaggactgcaagttgcctcttggtcaatctctctgtgctcttcctgacacacctagtactctcacacctcgcagtgtgcgctctgttccaggtgaaatgcgcttggcatccattgcttttaatcatcctattcaggttgatcaacttaatagtagttattttaaattaagtatacccactaatttttcctttggtgtgactcaggagtacattcagacaaccattcagaaagttactgttgattgtaaacagtacgtttgcaatggtttccagaagtgtgagcaattactgcgcgagtatggccagttttgttccaaaataaaccaggctctccatggtgccaatttacgccaggatgattctgtacgtaatttgtttgcgagcgtgaaaagctctcaatcatctcctatcataccaggttttggaggtgactttaatttgacacttctagaacctgtttctatatctactggcagtcgtagtgcacgtagtgctattgaggatttgctatttgacaaagtcactatagctgatcctggttatatgcaaggttacgatgattgcatgcagcaaggtccagcatcagctcgtgatcttatttgtgctcaatatgtggctggttacaaagtattacctcctcttatggatgttaatatggaagccgcgtatacttcatctttgcttggcagcatagcaggtgttggctggactgctggcttatcctcctttgctgctattccatttgcacagagtatcttttataggttaaacggtgttggcattactcaacaggttctttcagagaaccaaaagcttattgccaataagtttaatcaggctctgggagctatgcaaacaggcttcactacaactaatgaagcttttcagaaggttcaggatgctgtgaacaacaatgcacaggctctatccaaattagctagcgagctatctaatacttttggtgctatttccgcctctattggagacatcatacaacgtcttgatgttctcgaacaggacgcccaaatagacagacttattaatggccgtttgacaacactaaatgcttttgttgcacagcagcttgttcgttccgaatcagctgctctttccgctcaattggctaaagataaagtcaatgagtgtgtcaaggcacaatccaagcgttctggattttgcggtcaaggcacacatatagtgtcctttgttgtaaatgcccctaatggcctttacttcatgcatgttggttattaccctagcaaccacattgaggttgtttctgcttatggtctttgcgatgcagctaaccctactaattgtatagcccctgttaatggctactttattaaaactaataacactaggattgttgatgagtggtcatatactggctcgtccttctatgcacctgagcccattacctcccttaatactaagtatgttgcaccacaggtgacataccaaaacatttctactaacctccctcctcctcttctcggcaattccaccgggattgacttccaagatgagttggatgagtttttcaaaaatgttagcaccagtatacctaattttggttccctaacacagattaatactacattactcgatcttacctacgagatgttgtctcttcaacaagttgttaaagcccttaatgagtcttacatagaccttaaagagcttggcaattatacttattacaacaaatggccgtggtacatttggcttggtttcattgctgggcttgttgccttagctctatgcgtcttcttcatactgtgctgcactggttgtggcacaaactgtatgggaaaacttaagtgtaatcgttgttgtgatagatacgaggaatacgacctcgagccgcataaggttcatgttcactaattaacgaactattaatgagagttcaaagaccacccactctcttgttagtgttttcactctctcttttggtcactgcatcctcaaaacctctctatgtacctgagcattgtcagaattattctggttgcatgcttagggcttgtattaaaactgcccaagctgatacagctggtctttatacaaattttcgaattgacgtcccatctgcagaatcaactggtactcaatcagtttctgtcgatcttgagtcaacttcaactcatgatggtcctaccgaacatgttactagtgtgaatctttttgacgttggttactcagttaattaacgaactctatggattacgtgtctctgcttaatcaaatttggcagaagtaccttaactcaccgtatactacttgtttgtacatccctaaacccacagctaagtatacacctttagttggcacttcattgcaccctgtgctgtggaactgtcagctatcctttgctggttatactgaatctgctgttaattctacaaaagctttggccaaacaggacgcagctcagcgaatcgcttggttgctacataaggatggaggaatccctgatggatgttccctctacctccggcactcaagtttattcgcgcaaagcgaggaagaggagccattctccaactaagaaactgcgctacgttaagcgtagattttctcttctgcgccatgaagaccttagtgttattgtccaaccaacacactatgtcagggttacattttcagaccccaacatgtggtatctacgttcgggtcatcatttacactcagttcacaattggcttaaaccttatggcggccaacctgtttctgagtaccatattactctagctttgctaaatctcactgatgaagatttagctagagatttttcacccattgcgctctttttgcgcaatgtcagatttgagctacatgagttcgccttgctgcgcaaaactcttgttcttaatgcatcagagatctactgtgctaacatacatagatttaagcctgtgtatagagttaacacggcaatccctactattaaggattggcttctcgttcagggattttccctttaccatagtggcctccctttacatatgtcaatctctaaattgcatgcactggatgatgttactcgcaattacatcattacaatgccatgctttagaacttaccctcaacaaatgtttgttactcctttggccgtagatgttgtctccatacggtcttccaatcagggtaataaacaaattgttcattcttatcccattttacatcatccaggattttaacgaactatggctttctcggcgtctttatttaaacccgtccagctagtcccagtttctcctgcatttcatcgcattgagtctactgactctattgttttcacatacattcctgctagcggctatgtagctgctttagctgtcaatgtgtgtctcattcccctattattactgctacgtcaagatacttgtcgtcgcagcattatcagaactatggttctctatttccttgttctgtataactttttattagccattgtactagtcaatggtgtacattatccaactggaagttgcctgatagccttcttagttatcctcataatactttggtttgtagatagaattcgtttctgtctcatgctgaattcctacattccactgtttgacatgcgttcccactttattcgtgttagtacagtttcttctcatggtatggtccctgtaatacacaccaaaccattatttattagaaacttcgatcagcgttgcagctgttctcgttgtttttatttgcactcttccacttatatagagtgcacttatattagccgttttagtaagattagcctagtttctgtaactgacttctccttaaacggcaatgtttccactgttttcgtgcctgcaacgcgcgattcagttcctcttcacataatcgccccgagctcgcttatcgtttaagcagctctgcgctactatgggtcccgtgtagaggctaatccattagtctctctttggacatatggaaaacgaactatgttaccctttgtccaagaacgaatagggttgttcatagtaaactttttcatttttaccgtagtatgtgctataacactcttggtgtgtatggctttccttacggctactagattatgtgtgcaatgtatgacaggcttcaataccctgttagttcagcccgcattatacttgtataatactggacgttcagtctatgtaaaattccaggatagtaaaccccctctaccacctgacgagtgggtttaacgaactccttcataatgtctaatatgacgcaactcactgaggcgcagattattgccattattaaagactggaactttgcatggtccctgatctttctcttaattactatcgtactacagtatggatacccatcccgtagtatgactgtctatgtctttaaaatgtttgttttatggctcctatggccatcttccatggcgctatcaatatttagcgccgtttatccaattgatctagcttcccagataatctctggcattgtagcagctgtttcagctatgatgtggatttcctactttgtgcagagtatccggctgtttatgagaactggatcatggtggtcattcaatcctgagactaattgccttttgaacgttccatttggtggtacaactgtcgtacgtccactcgtagaggactctaccagtgtaactgctgttgtaaccaatggccacctcaaaatggctggcatgcatttcggtgcttgtgactacgacagacttcctaatgaagtcaccgtggccaaacccaatgtgctgattgctttaaaaatggtgaagcggcaaagctacggaactaattccggcgttgccatttaccatagatataaggcaggtaattacaggagtccgcctattacggcggatattgaacttgcattgcttcgagcttaggctctttagtaagagtatcttaattgattttaacgaatctcaatttcattgttatggcatcccctgctgcacctcgtgctgtttcctttgccgataacaatgatataacaaatacaaacctatctcgaggtagaggacgtaatccaaaaccacgagctgcaccaaataacactgtctcttggtacactgggcttacccaacacgggaaagtccctcttacctttccacctgggcagggtgtacctcttaatgccaattctacccctgcgcaaaatgctgggtattggcggagacaggacagaaaaattaataccgggaatggaattaagcaactggctcccaggtggtacttctactacactggaactggacccgaagcagcactcccattccgggctgttaaggatggcatcgtttgggtccatgaagatggcgccactgatgctccttcaacttttgggacgcggaaccctaacaatgattcagctattgttacacaattcgcgcccggtactaagcttcctaaaaacttccacattgaggggactggaggcaatagtcaatcatcttcaagagcctctagcttaagcagaaactcttccagatctagttcacaaggttcaagatcaggaaactctacccgcggcacttctccaggtccatctggaatcggagcagtaggaggtgatctactttaccttgatcttctgaacagactacaagcccttgagtctggcaaagtaaagcaatcgcagccaaaagtaatcactaagaaagatgctgctgctgctaaaaataagatgcgccacaagcgcacttccaccaaaagtttcaacatggtgcaagcttttggtcttcgcggaccaggagacctccagggaaactttggtgatcttcaattgaataaactcggcactgaggacccacgttggccccaaattgctgagcttgctcctacagccagtgcttttatgggtatgtcgcaatttaaacttacccatcagaacaatgatgatcatggcaaccctgtgtacttccttcggtacagtggagccattaaacttgacccaaagaatcccaactacaataagtggttggagcttcttgagcaaaatattgatgcctacaaaaccttccctaagaaggaaaagaaacaaaaggcaccaaaagaagaatcaacagaccaaatgtctgaacctccaaaggagcagcgtgtgcaaggtagcatcactcagcgcactcgcacccgtccaagtgttcagcctggtccaatgattgatgttaacactgattagtgtcactcaaagtaacaagatcgcggcaatcgtttgtgtttggcaaccccatctcaccatcgcttgtccactcttgcacagaatggaatcatgttgtaattacagtgcaataaggtaattataacccatttaattgatagctatgctttattaaagtgtgtagctgtagagagaatgttaaagactgtcacctctgcttgattgcaagtgaacagtgccccccgggaagagctctacagtgtgaaatgtaaataaaaaatagctattattcaattagattaggctaattagatgatttgcaaaaaaaaaaaa

SEQ ID NO: 3 - protein, 4391 aa, unidentified
MSFVAGVTAQGARGTYRAALNSEKHQDHVSLTVPLCGSGNLVEKLSPWFMDGENAYEVVKAMLLKKEPLLYVPIRLAGHTRHLPGPRVYLVERLIACENPFMVNQLAYSSSANGSLVGTTLQGKPIGMFFPYDIELVTGKQNILLRKYGRGGYHYTPFHYERDNTSCPEWMDDFEADPKGKYAQNLLKKLIGGDVTPVDQYMCGVDGKPISAYAFLMAKDGITKLADVEADVAARADDEGFITLKNNLYRLVWHVERKDVPYPKQSIFTINSVVQKDGVENTPPHYFTLGCKILTLTPRNKWSGVSDLSLKQKLLYTFYGKESLENPTYIYHSAFIECGSCGNDSWLTGNAIQGFACGCGASYTANDVEVQSSGMIKPNALLCATCPFAKGDSCSSNCKHSVAQLVSYLSERCNVIADSKSFTLIFGGVAYAYFGCEEGTMYFVPRAKSVVSRIGDSIFTGCTGSWNKVTQIANMFLEQTQHSLNFVGEFVVNDVVLAILSGTTTNVDKIRQLLKGVTLDKLRDYLADYDVAVTAGPFMDNAINVGGTGLQYAAITAPYVVLTGLGESFKKVATIPYKVCNSVKDTLAYYAHSVLYRVFPYDMDSGVSSFSELLFDCVDLSVASTYFLVRILQDKTGDFMSTIITSCQTAVSKLLDTCFEATEATFNFLLDLAGLFRIFLRNAYVYTSQGFVVVNGKVSTLVKQVLDLLNKGMQLLHTKVSWAGSKIIAVIYSGRESLIFPSGTYYCVTTKAKSVQQDLDVILPGEFSKKQLGLLQPTDNSTTVSVTVSSNMVETVVGQLEQTNMHSPDVIVGDYVIISEKLFVRSKEEDGFAFYPACTNGHAVPTLFRLKGGAPVKKVAFGGDQVHEVAAVRSVTVEYNIHAVLDTLLASSSLRTFVVDKSLSIEEFADVVKEQVSDLLVKLLRGMPIPDFDLDDFIDAPCYCFNAEGDASWSSTMIFSLHPVECDEECSEVEASDLEEGESECISETSTEQVDVSHETSDDEWAAAVDEAFPLDEAEDVTESVQEEAQPVEVPVEDIAQVVIADTLQETPVVPDTVEVPPQVVKLPSAPQTIQPEVKEVAPVYEADTEQTQNVTVKPKRLRKKRNVDPLSNFEHKVITECVTIVLGDAIQVAKCYGESVLVNAANTHLKHGGGIAGAINAASKGAVQKESDEYILAKGPLQVGDSVLLQGHSLAKNILHVVGPDARAKQDVSLLSKCYKAMNAYPLVVTPLVSAGIFGVKPAVSFDYLIREAKTRVLVVVNSQDVYKSLTIVDIPQSLTFSYDGLRGAIRKAKDYGFTVFVCTDNSANTKVLRNKGVDYTKKFLTVDGVQYYCYTSKDTLDDILQQANKSVGIISMPLGYVSHGLDLMQAGSVVRRVNVPYVCLLANKEQEAILMSEDVKLNPSEDFIKHVRTNGGYNSWHLVEGELLVQDLRLNKLLHWSDQTICYKDSVFYVVKNSTAFPFETLSACRAYLDSRTTQQLTIEVLVTVDGVNFRTVVLNNKNTYRSQLGCVFFNGADISDTIPDEKQNGHSLYLADNLTADETKALKELYGPVDPTFLHRFYSLKAAVHGWKMVVCDKVRSLKLSDNNCYLNAVIMTLDLLKDIKFVIPALQHAFMKHKGGDSTDFIALIMAYGNCTFGAPDDASRLLHTVLAKAELCCSARMVWREWCNVCGIKDVVLQGLKACCYVGVQTVEDLRARMTYVCQCGGERHRQLVEHTTPWLLLSGTPNEKLVTTSTAPDFVAFNVFQGIETAVGHYVHARLKGGLILKFDSGTVSKTSDWKCKVTDVLFPGQKYSSDCNVVRYSLDGNFRTEVDPDLSAFYVKDGKYFTSEPPVTYSPATILAGSVYTNSCLVSSDGQPGGDAISLSFNNLLGFDSSKPVTKKYTYSFLPKEDGDVLLAEFDTYDPIYKNGAMYKGKPILWVNKASYDTNLNKFNRASLRQIFDVAPIELENKFTPLSVESTPVEPPTVDVVALQQEMTIVKCKGLNKPFVKDNVSFVADDSGTPVVEYLSKEDLHTLYVDPKYQVIVLKDNVLSSMLRLHTVESGDINVVAASGSLTRKVKLLFRASFYFKEFATRTFTATTAVGSCIKSVVRHLGVTKGILTGCFSFAKMLFMLPLAYFSDSKLGTTEVKVSALKTAGVVTGNVVKQCCTAAVDLSMDKLRRVDWKSTLRLLLMLCTTMVLLSSVYHLYVFNQVLSSDVMFEDAQGLKKFYKEVRAYLGISSACDGLASAYRANSFDVPTFCANRSAMCNWCLISQDSITHYPALKMVQTHLSHYVLNIDWLWFAFETGLAYMLYTSAFNWLLLAGTLHYFFAQTSIFVDWRSYNYAVSSAFWLFTHIPMAGLVRMYNLLACLWLLRKFYQHVINGCKDTACLLCYKRNRLTRVEASTVVCGGKRTFYITANGGISFCRRHNWNCVDCDTAGVGNTFICEEVANDLTTALRRPINATDRSHYYVDSVTVKETVVQFNYRRDGQPFYERFPLCAFTNLDKLKFKEVCKTTTGIPEYNFIIYDSSDRGQESLARSACVYYSQVLCKSILLVDSSLVTSVGDSSEIATKMFDSFVNSFVSLYNVTRDKLEKLISTARDGVRRGDNFHSVLTTFIDAARGPAGVESDVETNEIVDSVQYAHKHDIQITNESYNNYVPSYVKPDSVSTSDLGSLIDCNAASVNQIVLRNSNGACIWNAAAYMKLSDALKRQIRIACRKCNLAFRLTTSKLRANDNILSVRFTANKIVGGAPTWFNALRDFTLKGYVLATIIVFLCAVLMYLCLPTFSMAPVEFYEDRILDFKVLDNGIIRDVNPDDKCFANKHRSFTQWYHEHVGGVYDNSITCPLTVAVIAGVAGARIPDVPTTLAWVNNQIIFFVSRVFANTGSVCYTPIDEIPYKSFSDSGCILPSECTMFRDAEGRMTPYCHDPTVLPGAFAYSQMRPHVRYDLYDGNMFIKFPEVVFESTLRITRTLSTQYCRFGSCEYAQEGVCITTNGSWAIFNDHHLNRPGVYCGSDFIDIVRRLAVSLFQPITYFQLTTSLVLGIGLCAFLTLLFYYINKVKRAFADYTQCAVIAVVAAVLNSLCICFVTSIPLCIVPYTALYYYATFYFTNEPAFIMHVSWYIMFGPIVPIWMTCVYTVAMCFRHFFWVLAYFSKKHVEVFTDGKLNCSFQDAASNIFVINKDTYAALRNSLTNDAYSRFLGLFNKYKYFSGAMETAAYREAAACHLAKALQTYSETGSDLLYQPPNCSITSGVLQSGLVKMSHPSGDVEACMVQVTCGSMTLNGLWLDNTVWCPRHVMCPADQLSDPNYDALLISMTNHSFSVQKHIGAPANLRVVGHAMQGTLLKLTVDVANPSTPAYTFTTVKPGAAFSVLACYNGRPTGTFTVVMRPNYTIKGSFLCGSCGSVGYTKEGSVINFCYMHQMELANGTHTGSAFDGTMYGAFMDKQVHQVQLTDKYCSVNVVAWLYAAILNGCAWFVKPNRTSVVSFNEWALANQFTEFVGTQSVDMLAVKTGVAIEQLLYAIQQLYTGFQGKQILGSTMLEDEFTPEDVNMQIMGVVMQSGVRKVTYGTAHWLFATLVSTYVIILQATKFTLWNYLFETIPTQLFPLLFVTMAFVMLLVKHKHTFLTLFLLPVAICLTYANIVYEPTTPISSALIAVANWLAPTNAYMRTTHTDIGVYISMSLVLVIVVKRLYNPSLSNFALALCSGVMWLYTYSIGEASSPIAYLVFVTTLTSDYTITVFVTVNLAKVCTYAIFAYSPQLTLVFPEVKMILLLYTCLGFMCTCYFGVFSLLNLKLRAPMGVYDFKVSTQEFRFMTANNLTAPRNSWEAMALNFKLIGIGGTPCIKVAAMQSKLTDLKCTSVVLLSVLQQLHLEANSRAWAFCVKCHNDILAATDPSEAFEKFVSLFATLMTFSGNVDLDALASDIFDTPSVLQATLSEFSHLATFAELEAAQKAYQEAMDSGDTSPQVLKALQKAVNIAKNAYEKDKAVARKLERMADQAMTSMYKQARAEDKKAKIVSAMQTMLFGMIKKLDNDVLNGIISNARNGCIPLSVIPLCASNKLRVVIPDFTVWNQVVTYPSLNYAGALWDITVINNVDNEIVKSSDVVDSNENLTWPLVLECTRASTSAVKLQNNEIKPSGLKTMVVSAGQEQTNCNTSSLAYYEPVQGRKMLMALLSDNAYLKWARVEGKDGFVSVELQPPCKFLIAGPKGPEIRYLYFVKNLNNLHRGQVLGHIAATVRLQAGSNTEFASNSSVLSLVNFTVDPQKAYLDFVNAGGAPLTNCVKMLTPKTGTGIAISVKPESTADQETYGGASVCLYCRAHIEHPDVSGVCKYKGKFVQIPAQCVRDPVGFCLSNTPCNVCQYWIGYGCNCDSLRQAALPQSKDSNFLNESGVLL

SEQ ID NO: 4 - protein, 7078 aa, unidentified
MSFVAGVTAQGARGTYRAALNSEKHQDHVSLTVPLCGSGNLVEKLSPWFMDGENAYEVVKAMLLKKEPLLYVPIRLAGHTRHLPGPRVYLVERLIACENPFMVNQLAYSSSANGSLVGTTLQGKPIGMFFPYDIELVTGKQNILLRKYGRGGYHYTPFHYERDNTSCPEWMDDFEADPKGKYAQNLLKKLIGGDVTPVDQYMCGVDGKPISAYAFLMAKDGITKLADVEADVAARADDEGFITLKNNLYRLVWHVERKDVPYPKQSIFTINSVVQKDGVENTPPHYFTLGCKILTLTPRNKWSGVSDLSLKQKLLYTFYGKESLENPTYIYHSAFIECGSCGNDSWLTGNAIQGFACGCGASYTANDVEVQSSGMIKPNALLCATCPFAKGDSCSSNCKHSVAQLVSYLSERCNVIADSKSFTLIFGGVAYAYFGCEEGTMYFVPRAKSVVSRIGDSIFTGCTGSWNKVTQIANMFLEQTQHSLNFVGEFVVNDVVLAILSGTTTNVDKIRQLLKGVTLDKLRDYLADYDVAVTAGPFMDNAINVGGTGLQYAAITAPYVVLTGLGESFKKVATIPYKVCNSVKDTLAYYAHSVLYRVFPYDMDSGVSSFSELLFDCVDLSVASTYFLVRILQDKTGDFMSTIITSCQTAVSKLLDTCFEATEATFNFLLDLAGLFRIFLRNAYVYTSQGFVVVNGKVSTLVKQVLDLLNKGMQLLHTKVSWAGSKIIAVIYSGRESLIFPSGTYYCVTTKAKSVQQDLDVILPGEFSKKQLGLLQPTDNSTTVSVTVSSNMVETVVGQLEQTNMHSPDVIVGDYVIISEKLFVRSKEEDGFAFYPACTNGHAVPTLFRLKGGAPVKKVAFGGDQVHEVAAVRSVTVEYNIHAVLDTLLASSSLRTFVVDKSLSIEEFADVVKEQVSDLLVKLLRGMPIPDFDLDDFIDAPCYCFNAEGDASWSSTMIFSLHPVECDEECSEVEASDLEEGESECISETSTEQVDVSHETSDDEWAAAVDEAFPLDEAEDVTESVQEEAQPVEVPVEDIAQVVIADTLQETPVVPDTVEVPPQVVKLPSAPQTIQPEVKEVAPVYEADTEQTQNVTVKPKRLRKKRNVDPLSNFEHKVITECVTIVLGDAIQVAKCYGESVLVNAANTHLKHGGGIAGAINAASKGAVQKESDEYILAKGPLQVGDSVLLQGHSLAKNILHVVGPDARAKQDVSLLSKCYKAMNAYPLVVTPLVSAGIFGVKPAVSFDYLIREAKTRVLVVVNSQDVYKSLTIVDIPQSLTFSYDGLRGAIRKAKDYGFTVFVCTDNSANTKVLRNKGVDYTKKFLTVDGVQYYCYTSKDTLDDILQQANKSVGIISMPLGYVSHGLDLMQAGSVVRRVNVPYVCLLANKEQEAILMSEDVKLNPSEDFIKHVRTNGGYNSWHLVEGELLVQDLRLNKLLHWSDQTICYKDSVFYVVKNSTAFPFETLSACRAYLDSRTTQQLTIEVLVTVDGVNFRTVVLNNKNTYRSQLGCVFFNGADISDTIPDEKQNGHSLYLADNLTADETKALKELYGPVDPTFLHRFYSLKAAVHGWKMVVCDKVRSLKLSDNNCYLNAVIMTLDLLKDIKFVIPALQHAFMKHKGGDSTDFIALIMAYGNCTFGAPDDASRLLHTVLAKAELCCSARMVWREWCNVCGIKDVVLQGLKACCYVGVQTVEDLRARMTYVCQCGGERHRQLVEHTTPWLLLSGTPNEKLVTTSTAPDFVAFNVFQGIETAVGHYVHARLKGGLILKFDSGTVSKTSDWKCKVTDVLFPGQKYSSDCNVVRYSLDGNFRTEVDPDLSAFYVKDGKYFTSEPPVTYSPATILAGSVYTNSCLVSSDGQPGGDAISLSFNNLLGFDSSKPVTKKYTYSFLPKEDGDVLLAEFDTYDPIYKNGAMYKGKPILWVNKASYDTNLNKFNRASLRQIFDVAPIELENKFTPLSVESTPVEPPTVDVVALQQEMTIVKCKGLNKPFVKDNVSFVADDSGTPVVEYLSKEDLHTLYVDPKYQVIVLKDNVLSSMLRLHTVESGDINVVAASGSLTRKVKLLFRASFYFKEFATRTFTATTAVGSCIKSVVRHLGVTKGILTGCFSFAKMLFMLPLAYFSDSKLGTTEVKVSALKTAGVVTGNVVKQCCTAAVDLSMDKLRRVDWKSTLRLLLMLCTTMVLLSSVYHLYVFNQVLSSDVMFEDAQGLKKFYKEVRAYLGISSACDGLASAYRANSFDVPTFCANRSAMCNWCLISQDSITHYPALKMVQTHLSHYVLNIDWLWFAFETGLAYMLYTSAFNWLLLAGTLHYFFAQTSIFVDWRSYNYAVSSAFWLFTHIPMAGLVRMYNLLACLWLLRKFYQHVINGCKDTACLLCYKRNRLTRVEASTVVCGGKRTFYITANGGISFCRRHNWNCVDCDTAGVGNTFICEEVANDLTTALRRPINATDRSHYYVDSVTVKETVVQFNYRRDGQPFYERFPLCAFTNLDKLKFKEVCKTTTGIPEYNFIIYDSSDRGQESLARSACVYYSQVLCKSILLVDSSLVTSVGDSSEIATKMFDSFVNSFVSLYNVTRDKLEKLISTARDGVRRGDNFHSVLTTFIDAARGPAGVESDVETNEIVDSVQYAHKHDIQITNESYNNYVPSYVKPDSVSTSDLGSLIDCNAASVNQIVLRNSNGACIWNAAAYMKLSDALKRQIRIACRKCNLAFRLTTSKLRANDNILSVRFTANKIVGGAPTWFNALRDFTLKGYVLATIIVFLCAVLMYLCLPTFSMAPVEFYEDRILDFKVLDNGIIRDVNPDDKCFANKHRSFTQWYHEHVGGVYDNSITCPLTVAVIAGVAGARIPDVPTTLAWVNNQIIFFVSRVFANTGSVCYTPIDEIPYKSFSDSGCILPSECTMFRDAEGRMTPYCHDPTVLPGAFAYSQMRPHVRYDLYDGNMFIKFPEVVFESTLRITRTLSTQYCRFGSCEYAQEGVCITTNGSWAIFNDHHLNRPGVYCGSDFIDIVRRLAVSLFQPITYFQLTTSLVLGIGLCAFLTLLFYYINKVKRAFADYTQCAVIAVVAAVLNSLCICFVTSIPLCIVPYTALYYYATFYFTNEPAFIMHVSWYIMFGPIVPIWMTCVYTVAMCFRHFFWVLAYFSKKHVEVFTDGKLNCSFQDAASNIFVINKDTYAALRNSLTNDAYSRFLGLFNKYKYFSGAMETAAYREAAACHLAKALQTYSETGSDLLYQPPNCSITSGVLQSGLVKMSHPSGDVEACMVQVTCGSMTLNGLWLDNTVWCPRHVMCPADQLSDPNYDALLISMTNHSFSVQKHIGAPANLRVVGHAMQGTLLKLTVDVANPSTPAYTFTTVKPGAAFSVLACYNGRPTGTFTVVMRPNYTIKGSFLCGSCGSVGYTKEGSVINFCYMHQMELANGTHTGSAFDGTMYGAFMDKQVHQVQLTDKYCSVNVVAWLYAAILNGCAWFVKPNRTSVVSFNEWALANQFTEFVGTQSVDMLAVKTGVAIEQLLYAIQQLYTGFQGKQILGSTMLEDEFTPEDVNMQIMGVVMQSGVRKVTYGTAHWLFATLVSTYVIILQATKFTLWNYLFETIPTQLFPLLFVTMAFVMLLVKHKHTFLTLFLLPVAICLTYANIVYEPTTPISSALIAVANWLAPTNAYMRTTHTDIGVYISMSLVLVIVVKRLYNPSLSNFALALCSGVMWLYTYSIGEASSPIAYLVFVTTLTSDYTITVFVTVNLAKVCTYAIFAYSPQLTLVFPEVKMILLLYTCLGFMCTCYFGVFSLLNLKLRAPMGVYDFKVSTQEFRFMTANNLTAPRNSWEAMALNFKLIGIGGTPCIKVAAMQSKLTDLKCTSVVLLSVLQQLHLEANSRAWAFCVKCHNDILAATDPSEAFEKFVSLFATLMTFSGNVDLDALASDIFDTPSVLQATLSEFSHLATFAELEAAQKAYQEAMDSGDTSPQVLKALQKAVNIAKNAYEKDKAVARKLERMADQAMTSMYKQARAEDKKAKIVSAMQTMLFGMIKKLDNDVLNGIISNARNGCIPLSVIPLCASNKLRVVIPDFTVWNQVVTYPSLNYAGALWDITVINNVDNEIVKSSDVVDSNENLTWPLVLECTRASTSAVKLQNNEIKPSGLKTMVVSAGQEQTNCNTSSLAYYEPVQGRKMLMALLSDNAYLKWARVEGKDGFVSVELQPPCKFLIAGPKGPEIRYLYFVKNLNNLHRGQVLGHIAATVRLQAGSNTEFASNSSVLSLVNFTVDPQKAYLDFVNAGGAPLTNCVKMLTPKTGTGIAISVKPESTADQETYGGASVCLYCRAHIEHPDVSGVCKYKGKFVQIPAQCVRDPVGFCLSNTPCNVCQYWIGYGCNCDSLRQAALPQSKDSNFLNRVRGSIVNARIEPCSSGLSTDVVFRAFDICNYKAKVAGIGKYYKTNTCRFVELDDQGHHLDSYFVVKRHTMENYELEKHCYDLLRDCDAVAPHDFFIFDVDKVKTPHIVRQRLTEYTMMDLVYALRHFDQNSEVLKAILVKYGCCDVTYFENKLWFDFVENPSVIGVYHKLGERVRQAILNTVKFCDHMVKAGLVGVLTLDNQDLNGKWYDFGDFVITQPGSGVAIVDSYYSYLMPVLSMTDCLAAETHRDCDFNKPLIEWPLTEYDFTDYKVQLFEKYFKYWDQTYHANCVNCTDDRCVLHCANFNVLFAMTMPKTCFGPIVRKIFVDGVPFVVSCGYHYKELGLVMNMDVSLHRHRLSLKELMMYAADPAMHIASSNAFLDLRTSCFSVAALTTGLTFQTVRPGNFNQDFYDFVVSKGFFKEGSSVTLKHFFFAQDGNAAITDYNYYSYNLPTMCDIKQMLFCMEVVNKYFEIYDGGCLNASEVVVNNLDKSAGHPFNKFGKARVYYESMSYQEQDELFAMTKRNVIPTMTQMNLKYAISAKNRARTVAGVSILSTMTNRQYHQKMLKSMAATRGATCVIGTTKFYGGWDFMLKTLYKDVDNPHLMGWDYPKCDRAMPNMCRIFASLILARKHGTCCTTRDRFYRLANECAQVLSEYVLCGGGYYVKPGGTSSGDATTAYANSVFNILQATTANVSALMGANGNKIVDKEVKDMQFDLYVNVYRSTSPDPKFVDKYYAFLNKHFSMMILSDDGVVCYNSDYAAKGYIAGIQNFKETLYYQNNVFMSEAKCWVETDLKKGPHEFCSQHTLYIKDGDDGYFLPYPDPSRILSAGCFVDDIVKTDGTLMVERFVSLAIDAYPLTKHEDIEYQNVFWVYLQYIEKLYKDLTGHMLDSYSVMLCGDNSAKFWEEAFYRDLYSSPTTLQAVGSCVVCHSQTSLRCGTCIRRPFLCCKCCYDHVIATPHKMVLSVSPYVCNAPGCGVSDVTKLYLGGMSYFCVDHRPVCSFPLCANGLVFGLYKNMCTGSPSIVEFNRLATCDWTESGDYTLANTTTEPLKLFAAETLRATEEASKQSYAIATIKEIVGERQLLLVWEAGKSKPPLNRNYVFTGYHITKNSKVQLGEYIFERIDYSDAVSYKSSTTYKLTVGDIFVLTSHSVATLTAPTIVNQERYVKITGLYPTITVPEEFASHVANFQKSGYSKYVTVQGPPGTGKSHFAIGLAIYYPTARVVYTACSHAAVDALCEKAFKYLNIAKCSRIIPAKARVECYDRFKVNETNSQYLFSTINALPETSADILVVDEVSMCTNYDLSIINARIKAKHIVYVGDPAQLPAPRTLLTRGTLEPENFNSVTRLMCNLGPDIFLSMCYRCPKEIVSTVSALVYNNKLLAKKELSGQCFKILYKGNVTHDASSAINRPQLTFVKNFITANPAWSKAVFISPYNSQNAVSRSMLGLTTQTVDSSQGSEYQYVIFCQTADTAHANNINRFNVAITRAQKGILCVMTSQALFESLEFTELSFTNYKLQSQIVTGLFKDCSRETSGLSPAYAPTYVSVDDKYKTSDELCVNLNLPANVPYSRVISRMGFKLDATVPGYPKLFITREEAVRQVRSWIGFDVEGAHASRNACGTNVPLQLGFSTGVNFVVQPVGVVDTEWGNMLTGIAARPPPGEQFKHLVPLMHKGAAWPIVRRRIVQMLSDTLDKLSDYCTFVCWAHGFELTSASYFCKIGKEQKCCMCNRRAAAYSSPLQSYACWTHSCGYDYVYNPFFVDVQQWGYVGNLATNHDRYCSVHQGAHVASNDAIMTRCLAIHSCFIERVDWDIEYPYISHEKKLNSCCRIVERNVVRAALLAGSFDKVYDIGNPKGIPIVDDPVVDWHYFDAQPLTRKVQQLFYTEDMASRFADGLCLFWNCNVPKYPNNAIVCRFDTRVHSEFNLPGCDGGSLYVNKHAFHTPAYDVSAFRDLKPLPFFYYSTTPCEVHGNGSMIEDIDYVPLKSAVCITACNLGGAVCRKHATEYREYMEAYNLVSASGFRLWCYKTFDIYNLWSTFTKVQGLENIAFNVVKQGHFIGVEGELPVAVVNDKIFTKSGVNDICMFENKTTLPTNIAFELYAKRAVRSHPDFKLLHNLQADICYKFVLWDYERSNIYGTATIGVCKYTDIDVNSALNICFDIRDNCSLEKFMSTPNAIFISDRKIKKYPCMVGPDYAYFNGAIIRDSDVVKQPVKFYLYKKVNNEFIDPTECIYTQSRSCSDFLPLSDMEKDFLSFDSDVFIKKYGLENYAFEHVVYGDFSHTTLGGLHLLIGLYKKQQEGHIIMEEMLKGSSTIHNYFITETNTAAFKAVCSVIDLKLDDFVMILKSQDLGVVSKVVKVPIDLTMIEFMLWCKDGQVQTFYPRLQASADWKPGHAMPSLFKVQNVNLERCELANYKQSIPMPRGVHMNIAKYMQLCQYLNTCTLAVPANMRVIHFGAGSDKGIAPGTSVLRQWLPTDAIIIDNDLNEFVSDADITLFGDCVTVRVGQQVDLVISDMYDPTTKNVTGSNESKALFFTYLCNLINNNLALGGSVAIKITEHSWSVELYELMGKFAWWTVFCTNANASSSEGFLLGINYLGTIKENIDGGAMHANYIFWRNSTPMNLSTYSLFDLSKFQLKLKGTPVLQLKESQINELVISLLSQGKLLIRDNDTLSVSTDVLVNTYRKLR

SEQ ID NO: 5 - protein, 1353 aa, unidentified
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFQKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 6 - protein, 103 aa, unidentified
MRVQRPPTLLLVFSLSLLVTASSKPLYVPEHCQNYSGCMLRACIKTAQADTAGLYTNFRIDVPSAESTGTQSVSVDLESTSTHDGPTEHVTSVNLFDVGYSVN

SEQ ID NO: 7 - protein, 109 aa, unidentified
MDYVSLLNQIWQKYLNSPYTTCLYIPKPTAKYTPLVGTSLHPVLWNCQLSFAGYTESAVNSTKALAKQDAAQRIAWLLHKDGGIPDGCSLYLRHSSLFAQSEEEEPFSN

SEQ ID NO: 8 - protein, 246 aa, unidentified
MEESLMDVPSTSGTQVYSRKARKRSHSPTKKLRYVKRRFSLLRHEDLSVIVQPTHYVRVTFSDPNMWYLRSGHHLHSVHNWLKPYGGQPVSEYHITLALLNLTDEDLARDFSPIALFLRNVRFELHEFALLRKTLVLNASEIYCANIHRFKPVYRVNTAIPTIKDWLLVQGFSLYHSGLPLHMSISKLHALDDVTRNYIITMPCFRTYPQQMFVTPLAVDVVSIRSSNQGNKQIVHSYPILHHPGF

SEQ ID NO: 9 - protein, 224 aa, unidentified
MAFSASLFKPVQLVPVSPAFHRIESTDSIVFTYIPASGYVAALAVNVCLIPLLLLLRQDTCRRSIIRTMVLYFLVLYNFLLAIVLVNGVHYPTGSCLIAFLVILIILWFVDRIRFCLMLNSYIPLFDMRSHFIRVSTVSSHGMVPVIHTKPLFIRNFDQRCSCSRCFYLHSSTYIECTYISRFSKISLVSVTDFSLNGNVSTVFVPATRDSVPLHIIAPSSLIV

SEQ ID NO: 10 - protein, 82 aa, unidentified
MLPFVQERIGLFIVNFFIFTVVCAITLLVCMAFLTATRLCVQCMTGFNTLLVQPALYLYNTGRSVYVKFQDSKPPLPPDEWV

SEQ ID NO: 11 - protein, 219 aa, unidentified
MSNMTQLTEAQIIAIIKDWNFAWSLIFLLITIVLQYGYPSRSMTVYVFKMFVLWLLWPSSMALSIFSAVYPIDLASQIISGIVAAVSAMMWISYFVQSIRLFMRTGSWWSFNPETNCLLNVPFGGTTVVRPLVEDSTSVTAVVTNGHLKMAGMHFGACDYDRLPNEVTVAKPNVLIALKMVKRQSYGTNSGVAIYHRYKAGNYRSPPITADIELALLRA

SEQ ID NO: 12 - protein, 413 aa, unidentified
MASPAAPRAVSFADNNDITNTNLSRGRGRNPKPRAAPNNTVSWYTGLTQHGKVPLTFPPGQGVPLNANSTPAQNAGYWRRQDRKINTGNGIKQLAPRWYFYYTGTGPEAALPFRAVKDGIVWVHEDGATDAPSTFGTRNPNNDSAIVTQFAPGTKLPKNFHIEGTGGNSQSSSRASSLSRNSSRSSSQGSRSGNSTRGTSPGPSGIGAVGGDLLYLDLLNRLQALESGKVKQSQPKVITKKDAAAAKNKMRHKRTSTKSFNMVQAFGLRGPGDLQGNFGDLQLNKLGTEDPRWPQIAELAPTASAFMGMSQFKLTHQNNDDHGNPVYFLRYSGAIKLDPKNPNYNKWLELLEQNIDAYKTFPKKEKKQKAPKEESTDQMSEPPKEQRVQGSITQRTRTRPSVQPGPMIDVNTD

SEQ ID NO: 13 - protein, 112 aa, unidentified
MPILPLRKMLGIGGDRTEKLIPGMELSNWLPGGTSTTLELDPKQHSHSGLLRMASFGSMKMAPLMLLQLLGRGTLTMIQLLLHNSRPVLSFLKTSTLRGLEAIVNHLQEPLA

SEQ ID NO: 14 - protein, 4391 aa, unidentified
MSFVAGVTAQGARGTYRAALNSEKHQDHVSLTVPLCGSGNLVEKLSPWFMDGENAYEVVKAMLLKKEPLLYVPIRLAGRTRHLPGPRVYLVERLIACENPFMVNQLAYSSSANGSLVGTTLQGKPIGMFFPYDIELVTGKQNILLRKYGRGGYHYTPFHYERDNTSCPEWMDDFEADPKGKYAQNLLKKLIGGDVTPVDQYMCGVDGKPISAYAFLMAKDGITKLADVEADVAARADDEGFITLKNNLYRLVWHVERKDVPYPKQSIFTINSVVQKDGVENTPPHYFTLGCKILTLTPRNKWSGVSDLSLKQKLLYTFYGKESLENPTYIYHSAFIECGSCGNDSWLTGNAIQGFACGCGASYTANDVEVQSSGMIKPNALLCATCPFAKGDSCSSNCKHSVAQLVSYLSERCNVIADSKSFTLIFGGVAYAYFGCEEGTMYFVPRAKSVVSRIGDSIFTGCTGSWNKVTQIANMFLEQTQHSLNFVGEFVVNDVVLAILSGTTTNVDKIRQLLKGVTLDKLRDYLADYDVAVTAGPFMDNAINVGGTGLQYAAITAPYVVLTGLGESFKKVATIPYKVCNSVKDTLAYYAHSVLYRVFPYDMDSGVSSFSELLFDCVDLSVASTYFLVRILQDKTGDFMSTIITSCQTAVSKLLDTCFEATEATFNFLLDLAGLFRIFLRNAYVYTSQGFVVVNGKVSTLVKQVLDLLNKGMQLLHTKVSWAGSKIIAVIYSGRESLIFPSGTYYCVTTKAKSVQQDLDVILPGEFSKKQLGLLQPTDNSTTVSVTVSSNMVETVVGQLEQTNMHSPDVIVGDYVIISEKLFVRSKEEDGFAFYPACTNGHAVPTLFRLKGGAPVKKVAFGGDQVHEVAAVRSVTVEYNIHAVLDTLLASSSLRTFVVDKSLSIEEFADVVKEQVSDLLVKLLRGMPIPDFDLDDFIDAPCYCFNAEGDASWSSTMIFSLHPVECDEECSEVEASDLEEGESECISETSTEQVDVSHETSDDEWAAAVDEAFPLDEAEDVTESVQEEAQPVEVPVEDIAQVVIADTLQETPVVPDTVEVPPQVVKLPSAPQTIQPEVKEVAPVYEADTEQTQNVTVKPKRLRKKRNVDPLSNFEHKVITECVTIVLGDAIQVAKCYGESVLVNAANTHLKHGGGIAGAINAASKGAVQKESDEYILAKGPLQVGDSVLLQGHSLAKNILHVVGPDARAKQDVSLLSKCYKAMNAYPLVVTPLVSAGIFGVKPAVSFDYLIREAKTRVLVVVNSQDVYKSLTIVDIPQSLTFSYDGLRGAIRKAKDYGFTVFVCTDNSANTKVLRNKGVDYTKKFLTVDGVQYYCYTSKDTLDDILQQANKSVGIISMPLGYVSHGLDLMQAGSVVRRVNVPYVCLLANKEQEAILMSEDVKLNPSEDFIKHVRTNGGYNSWHLVEGELLVQDLRLNKLLHWSDQTICYKDSVFYVVKNSTAFPFETLSACRAYLDSRTTQQLTIEVLVTVDGVNFRTVVLNNKNTYRSQLGCVFFNGADISDTIPDEKQNGHSLYLADNLTADETKALKELYGPVDPTFLHRFYSLKAAVHGWKMVVCDKVRSLKLSDNNCYLNAVIMTLDLLKDIKFVIPALQLAFMKHKGGDSTDFIALIMAYGNCTFGAPDDASRLLHTVLAKAELCCSARMVWREWCNVCGIKDVVLQGLKACCYVGVQTVEDLRARMTYVCQCGGERHRQLVEHTTPWLLLSGTPNEKLVTTSTAPDFVAFNVFQGIETAVGHYVHARLKGGLILKFDSGTVSKTSDWKCKVTDVLFPGQKYSSDCNVVRYSLDGNFRTEVDPDLSAFYVKDGKYFTSEPPVTYSPATILAGSVYTNSCLVSSDGQPGGDAISLSFNNLLGFDSSKPVTKKYTYSFLPKEDGDVLLAEFDTYDPIYKNGAMYKGKPILWVNKASYDTNLNKFNRASLRQIFDVAPIELENKFTPLSVESTPVEPPTVDVVALQQEMTIVKCKGLNKPFVKDNVSFVADDSGTPVVEYLSKEDLHTLYVDPKYQVIVLKDNVLSSMLRLHTVESGDINVVAASGSLTRKVKLLFRASFYFKEFATRTFTATPAVGSCIKSVVRHLGVTKGILTGCFSFAKMLFMLPLAYFSDSKLGTTEVKVSALKTAGVVTGNVVKQCCTAAVDLSMDKLRRVDWKSTLRLLLMLCTTMVLLSSVYHLYVFNQVLSSDVMFEDVQGLKKFYKEVRAYLGISSACDGLASAYRANSFDVPTFCANRSAMCNWCLISQDSITHYPALKMVQTHLSHYVLNIDWLWFAFETGLAYMLYTSAFNWLLLAGTLHYFFAQTSIFVDWRSYNYAVSSAFWLFTHIPMAGLVRMYNLLACLWLLRKFYQHVINGCKDTACLLCYKRNRLTRVEASTVVCGGKRTFYITANGGISFCRRHNWNCVDCDTAGVGNTFICEEVANDLTTALRRPINATDRSHYYVDSVTVKETVVQFNYRRDGQPFYERFPLCAFTNLDKLKFKEVCKTTTGIPEYNFIIYDSSDRGQESLARSACVYYSQVLCKSILLVDSSLVTSVGDSSEIATKMFDSFVNSFVSLYNVTRDKLEKLISTARDGVRRGDNFHSVLTTFIDAARGPAGVESDVETNEIVDSVQYAHKHDIQITNESYNNYVPSYVKPDSVSTSDLGSLIDCNAASVNQIVLRNSNGACIWNAAAYMKLSDALKRQIRIACRKCNLAFRLTTSKLRANDNILSVRFTANKIVGGAPTWFNALRDFTLKGYVLATIIVFLCAVLMYLCLPTFSMAPVEFYEDRILDFKVLDNGIIRDVNPDDKCFANKHRSFTQWYHEHVGGVYDNSITCPLTVAVIAGVAGARIPDVPTTLAWVNNQIIFFVSRVFANTGSVCYTPIDEIPYKSFSDSGCILPSECTMFRDAEGRMTPYCHDPTVLPGAFAYSQMRPHVRYDLYDGNMFIKFPEVVFESTLRITRTLSTQYCRFGSCEYAQEGVCITTNGSWAIFNDHHLNRPGVYCGSDFIDIVRRLAVSLFQPITYFQLTTSLVLGIGLCAFLTLLFYYINKVKRAFADYTQCAVIAVVAAVLNSLCICFVTSIPLCIVPYTALYYYATFYFTNEPAFIMHVSWYIMFGPIVPIWMTCVYTVAMCFRHFFWVLAYFSKKHVEVFTDGKLNCSFQDAASNIFVINKDTYAALRNSLTNDAYSRFLGLFNKYKYFSGAMETAAYREAAACHLAKALQTYSETGSDLLYQPPNCSITSGVLQSGLVKMSHPSGDVEACMVQVTCGSMTLNGLWLDNTVWCPRHVMCPADRLSDPNYDALLISMTNHSFSVQKHIGAPANLRVVGHAMQGTLLKLTVDVANPSTPAYTFTTVKPGAAFSVLACYNGRPTGTFTVVMRPNYTIKGSFLCGSCGSVGYTKEGSVINFCYMHQMELANGTHTGSAFDGTMYGAFMDKQVHQVQLTDKYCSVNVVAWLYAAILNGCAWFVKPNRTSVVSFNEWALANQFTEFVGTQSVDMLAVKTGVAIEQLLYAIQQLYTGFQGKQILGSTMLEDEFTPEDVNMQIMGVVMQSGVRKVTYGTAHWLFATLVSTYVIILQATKFTLWNYLFETIPTQLFPLLFVTMAFVMLLVKHKHTFLTLFLLPVAICLTYANIVYEPTTPISSALIAVANWLAPTNAYMRTTHTDIGVYISMSLVLVIVVKRLYNPSLSNFALALCSGVMWLYTYSIGEASSPIAYLVFVTTLTSDYTITVIVTVNLAKVCTYAIFAYSPQLTLVFPEVKMILLLYTCLGFMCTCYFGVFSLLNLKLRAPMGVYDFKVSTQEFRFMTANNLTAPRNSWGAMALNFKLIGIGGTPCIKVAAMQSKLTDLKCTSVVLLSVLQQLHLEANSRAWAFCVKCHNDILAATDPSEAFEKFVSLFATLMTFSGNVDLDALASDIFDTPSVLQATLSEFSHLATFAELEAAQKAYQEAMDSGDTSPQVLKALQKAVNIAKNAYEKDKAVARKLERMADQAMTSMYKQARAEDKKAKIVSAMQTMLFGMIKKLDNDVLNGIISNARNGCIPLSVIPLCASNKLRVVIPDFTVWNQVVTYPSLNYAGALWDITVINNVDNEIVKSSDVVDSNENLTWPLVLECTRASTSAVKLQNNEIKPSGLKTMVVSAGQEQTNCNTSSLAYYEPVQGRKMLMALLSDNAYLKWARVEGKDGFVSVELQSPCKFLIAGPKGPEIRYLYFVKNLNNLHRGQVLGHIAATVRLQAGSNTEFASSSSVLSLVNFTVDPQKAYLDFVNAGGAPLTNCVKMLTPKTGTGIAISVKPESTADQETYGGASVCLYCRAHIEHPDVSGVCKYKGKFVQIPAQCVRDPVGFCLSNTPCNVCQYWIGYGCNCDSLRQAALPQSKDSNFLNESGVLL

SEQ ID NO: 15 - protein, 7078 aa, unidentified
MSFVAGVTAQGARGTYRAALNSEKHQDHVSLTVPLCGSGNLVEKLSPWFMDGENAYEVVKAMLLKKEPLLYVPIRLAGRTRHLPGPRVYLVERLIACENPFMVNQLAYSSSANGSLVGTTLQGKPIGMFFPYDIELVTGKQNILLRKYGRGGYHYTPFHYERDNTSCPEWMDDFEADPKGKYAQNLLKKLIGGDVTPVDQYMCGVDGKPISAYAFLMAKDGITKLADVEADVAARADDEGFITLKNNLYRLVWHVERKDVPYPKQSIFTINSVVQKDGVENTPPHYFTLGCKILTLTPRNKWSGVSDLSLKQKLLYTFYGKESLENPTYIYHSAFIECGSCGNDSWLTGNAIQGFACGCGASYTANDVEVQSSGMIKPNALLCATCPFAKGDSCSSNCKHSVAQLVSYLSERCNVIADSKSFTLIFGGVAYAYFGCEEGTMYFVPRAKSVVSRIGDSIFTGCTGSWNKVTQIANMFLEQTQHSLNFVGEFVVNDVVLAILSGTTTNVDKIRQLLKGVTLDKLRDYLADYDVAVTAGPFMDNAINVGGTGLQYAAITAPYVVLTGLGESFKKVATIPYKVCNSVKDTLAYYAHSVLYRVFPYDMDSGVSSFSELLFDCVDLSVASTYFLVRILQDKTGDFMSTIITSCQTAVSKLLDTCFEATEATFNFLLDLAGLFRIFLRNAYVYTSQGFVVVNGKVSTLVKQVLDLLNKGMQLLHTKVSWAGSKIIAVIYSGRESLIFPSGTYYCVTTKAKSVQQDLDVILPGEFSKKQLGLLQPTDNSTTVSVTVSSNMVETVVGQLEQTNMHSPDVIVGDYVIISEKLFVRSKEEDGFAFYPACTNGHAVPTLFRLKGGAPVKKVAFGGDQVHEVAAVRSVTVEYNIHAVLDTLLASSSLRTFVVDKSLSIEEFADVVKEQVSDLLVKLLRGMPIPDFDLDDFIDAPCYCFNAEGDASWSSTMIFSLHPVECDEECSEVEASDLEEGESECISETSTEQVDVSHETSDDEWAAAVDEAFPLDEAEDVTESVQEEAQPVEVPVEDIAQVVIADTLQETPVVPDTVEVPPQVVKLPSAPQTIQPEVKEVAPVYEADTEQTQNVTVKPKRLRKKRNVDPLSNFEHKVITECVTIVLGDAIQVAKCYGESVLVNAANTHLKHGGGIAGAINAASKGAVQKESDEYILAKGPLQVGDSVLLQGHSLAKNILHVVGPDARAKQDVSLLSKCYKAMNAYPLVVTPLVSAGIFGVKPAVSFDYLIREAKTRVLVVVNSQDVYKSLTIVDIPQSLTFSYDGLRGAIRKAKDYGFTVFVCTDNSANTKVLRNKGVDYTKKFLTVDGVQYYCYTSKDTLDDILQQANKSVGIISMPLGYVSHGLDLMQAGSVVRRVNVPYVCLLANKEQEAILMSEDVKLNPSEDFIKHVRTNGGYNSWHLVEGELLVQDLRLNKLLHWSDQTICYKDSVFYVVKNSTAFPFETLSACRAYLDSRTTQQLTIEVLVTVDGVNFRTVVLNNKNTYRSQLGCVFFNGADISDTIPDEKQNGHSLYLADNLTADETKALKELYGPVDPTFLHRFYSLKAAVHGWKMVVCDKVRSLKLSDNNCYLNAVIMTLDLLKDIKFVIPALQLAFMKHKGGDSTDFIALIMAYGNCTFGAPDDASRLLHTVLAKAELCCSARMVWREWCNVCGIKDVVLQGLKACCYVGVQTVEDLRARMTYVCQCGGERHRQLVEHTTPWLLLSGTPNEKLVTTSTAPDFVAFNVFQGIETAVGHYVHARLKGGLILKFDSGTVSKTSDWKCKVTDVLFPGQKYSSDCNVVRYSLDGNFRTEVDPDLSAFYVKDGKYFTSEPPVTYSPATILAGSVYTNSCLVSSDGQPGGDAISLSFNNLLGFDSSKPVTKKYTYSFLPKEDGDVLLAEFDTYDPIYKNGAMYKGKPILWVNKASYDTNLNKFNRASLRQIFDVAPIELENKFTPLSVESTPVEPPTVDVVALQQEMTIVKCKGLNKPFVKDNVSFVADDSGTPVVEYLSKEDLHTLYVDPKYQVIVLKDNVLSSMLRLHTVESGDINVVAASGSLTRKVKLLFRASFYFKEFATRTFTATPAVGSCIKSVVRHLGVTKGILTGCFSFAKMLFMLPLAYFSDSKLGTTEVKVSALKTAGVVTGNVVKQCCTAAVDLSMDKLRRVDWKSTLRLLLMLCTTMVLLSSVYHLYVFNQVLSSDVMFEDVQGLKKFYKEVRAYLGISSACDGLASAYRANSFDVPTFCANRSAMCNWCLISQDSITHYPALKMVQTHLSHYVLNIDWLWFAFETGLAYMLYTSAFNWLLLAGTLHYFFAQTSIFVDWRSYNYAVSSAFWLFTHIPMAGLVRMYNLLACLWLLRKFYQHVINGCKDTACLLCYKRNRLTRVEASTVVCGGKRTFYITANGGISFCRRHNWNCVDCDTAGVGNTFICEEVANDLTTALRRPINATDRSHYYVDSVTVKETVVQFNYRRDGQPFYERFPLCAFTNLDKLKFKEVCKTTTGIPEYNFIIYDSSDRGQESLARSACVYYSQVLCKSILLVDSSLVTSVGDSSEIATKMFDSFVNSFVSLYNVTRDKLEKLISTARDGVRRGDNFHSVLTTFIDAARGPAGVESDVETNEIVDSVQYAHKHDIQITNESYNNYVPSYVKPDSVSTSDLGSLIDCNAASVNQIVLRNSNGACIWNAAAYMKLSDALKRQIRIACRKCNLAFRLTTSKLRANDNILSVRFTANKIVGGAPTWFNALRDFTLKGYVLATIIVFLCAVLMYLCLPTFSMAPVEFYEDRILDFKVLDNGIIRDVNPDDKCFANKHRSFTQWYHEHVGGVYDNSITCPLTVAVIAGVAGARIPDVPTTLAWVNNQIIFFVSRVFANTGSVCYTPIDEIPYKSFSDSGCILPSECTMFRDAEGRMTPYCHDPTVLPGAFAYSQMRPHVRYDLYDGNMFIKFPEVVFESTLRITRTLSTQYCRFGSCEYAQEGVCITTNGSWAIFNDHHLNRPGVYCGSDFIDIVRRLAVSLFQPITYFQLTTSLVLGIGLCAFLTLLFYYINKVKRAFADYTQCAVIAVVAAVLNSLCICFVTSIPLCIVPYTALYYYATFYFTNEPAFIMHVSWYIMFGPIVPIWMTCVYTVAMCFRHFFWVLAYFSKKHVEVFTDGKLNCSFQDAASNIFVINKDTYAALRNSLTNDAYSRFLGLFNKYKYFSGAMETAAYREAAACHLAKALQTYSETGSDLLYQPPNCSITSGVLQSGLVKMSHPSGDVEACMVQVTCGSMTLNGLWLDNTVWCPRHVMCPADRLSDPNYDALLISMTNHSFSVQKHIGAPANLRVVGHAMQGTLLKLTVDVANPSTPAYTFTTVKPGAAFSVLACYNGRPTGTFTVVMRPNYTIKGSFLCGSCGSVGYTKEGSVINFCYMHQMELANGTHTGSAFDGTMYGAFMDKQVHQVQLTDKYCSVNVVAWLYAAILNGCAWFVKPNRTSVVSFNEWALANQFTEFVGTQSVDMLAVKTGVAIEQLLYAIQQLYTGFQGKQILGSTMLEDEFTPEDVNMQIMGVVMQSGVRKVTYGTAHWLFATLVSTYVIILQATKFTLWNYLFETIPTQLFPLLFVTMAFVMLLVKHKHTFLTLFLLPVAICLTYANIVYEPTTPISSALIAVANWLAPTNAYMRTTHTDIGVYISMSLVLVIVVKRLYNPSLSNFALALCSGVMWLYTYSIGEASSPIAYLVFVTTLTSDYTITVIVTVNLAKVCTYAIFAYSPQLTLVFPEVKMILLLYTCLGFMCTCYFGVFSLLNLKLRAPMGVYDFKVSTQEFRFMTANNLTAPRNSWGAMALNFKLIGIGGTPCIKVAAMQSKLTDLKCTSVVLLSVLQQLHLEANSRAWAFCVKCHNDILAATDPSEAFEKFVSLFATLMTFSGNVDLDALASDIFDTPSVLQATLSEFSHLATFAELEAAQKAYQEAMDSGDTSPQVLKALQKAVNIAKNAYEKDKAVARKLERMADQAMTSMYKQARAEDKKAKIVSAMQTMLFGMIKKLDNDVLNGIISNARNGCIPLSVIPLCASNKLRVVIPDFTVWNQVVTYPSLNYAGALWDITVINNVDNEIVKSSDVVDSNENLTWPLVLECTRASTSAVKLQNNEIKPSGLKTMVVSAGQEQTNCNTSSLAYYEPVQGRKMLMALLSDNAYLKWARVEGKDGFVSVELQSPCKFLIAGPKGPEIRYLYFVKNLNNLHRGQVLGHIAATVRLQAGSNTEFASSSSVLSLVNFTVDPQKAYLDFVNAGGAPLTNCVKMLTPKTGTGIAISVKPESTADQETYGGASVCLYCRAHIEHPDVSGVCKYKGKFVQIPAQCVRDPVGFCLSNTPCNVCQYWIGYGCNCDSLRQAALPQSKDSNFLNRVRGSIVNARIEPCSSGLSTDVVFRAFDICNYKAKVAGIGKYYKTNTCRFVELDDQGHHLDSYFVVKRHTMENYELEKHCYDLLRDCDAVAPHDFFIFDVDKVKTPHIVRQRLTEYTMMDLVYALRHFDQNSEVLKAILVKYGCCDVTYFENKLWFDFVENPSVIGVYHKLGERVRQAILNTVKFCDHMVKAGLVGVLTLDNQDLNGKWYDFGDFVITQPGSGVAIVDSYYSYLMPVLSMTDCLAAETHRDCDFNKPLIEWPLTEYDFTDYKVQLFEKYFKYWDQTYHANCVNCTDDRCVLHCANFNVLFAMTMPKTCFGPIVRKIFVDGVPFVVSCGYHYKELGLVMNMDVSLHRHRLSLKELMMYAADPAMHIASSNAFLDLRTSCFSVAALTTGLTFQTVRPGNFNQDFYDFVVSKGFFKEGSSVTLKHFFFAQDGNAAITDYNYYSYNLPTMCDIKQMLFCMEVVNKYFEIYDGGCLNASEVVVNNLDKSAGHPFNKFGKARVYYESMSYQEQDELFAMTKRNVIPTMTQMNLKYAISAKNRARTVAGVSILSTMTNRQYHQKMLKSMAATRGATCVIGTTKFYGGWDFMLKTLYKDVDNPHLMGWDYPKCDRAMPNMCRIFASLILARKHGTCCTTRDRFYRLANECAQVLSEYVLCGGGYYVKPGGTSSGDATTAYANSVFNILQATTANVSALMGANGNKIVDKEVKDMQFDLYVNVYRSTSPDPKFVDKYYAFLNKHFSMMILSDDGVVCYNSDYAAKGYIAGIQNFKETLYYQNNVFMSEAKCWVETDLKKGPHEFCSQHTLYIKDGDDGYFLPYPDPSRILSAGCFVDDIVKTDGTLMVERFVSLAIDAYPLTKHEDIEYQNVFWVYLQYIEKLYKDLTGHMLDSYSVMLCGDNSAKFWEEAFYRDLYSSPTTLQAVGSCVVCHSQTSLRCGTCIRRPFLCCKCCYDHVIATPHKMVLSVSPYVCNAPGCGVSDVTKLYLGGMSYFCVDHRPVCSFPLCANGLVFGLYKNMCTGSPSIVEFNRLATCDWTESGDYTLANTTTEPLKLFAAETLRATEEASKQSYAIATIKEIVGERQLLLVWEAGKSKPPLNRNYVFTGYHITKNSKVQLGEYIFERIDYSDAVSYKSSTTYKLTVGDIFVLTSHSVATLTAPTIVNQERYVKITGLYPTITVPEEFASHVANFQKSGYSKYVTVQGPPGTGKSHFAIGLAIYYPTARVVYTACSHAAVDALCEKAFKYLNIAKCSRIIPAKARVECYDRFKVNETNSQYLFSTINALPETSADILVVDEVSMCTNYDLSIINARIKAKHIVYVGDPAQLPAPRTLLTRGTLEPENFNSVTRLMCNLGPDIFLSMCYRCPKEIVSTVSALVYNNKLLAKKELSGQCFKILYKGNVTHDASSAINRPQLTFVKNFITANPAWSKAVFISPYNSQNAVSRSMLGLTTQTVDSSQGSEYQYVIFCQTADTAHANNINRFNVAITRAQKGILCVMTSQALFESLEFTELSFTNYKLQSQIVTGLFKDCSRETSGLSPAYAPTYVSVDDKYKTSDELCVNLNLPANVPYSRVISRMGFKLDATVPGYPKLFITREEAVRQVRSWIGFDVEGAHASRNACGTNVPLQLGFSTGVNFVVQPVGVVDTEWGNMLTGIAARPPPGEQFKHLVPLMHKGAAWPIVRRRIVQMLSDTLDKLSDYCTFVCWAHGFELTSASYFCKIGKEQKCCMCNRRAAAYSSPLQSYACWTHSCGYDYVYNPFFVDVQQWGYVGNLATNHDRYCSVHQGAHVASNDAIMTRCLAIHSCFIERVDWDIEYPYISHEKKLNSCCRIVERNVVRAALLAGSFDKVYDIGNPKGIPIVDDPVVDWHYFDAQPLTRKVQQLFYTEDMASRFADGLCLFWNCNVPKYPNNAIVCRFDTRVHSEFNLPGCDGGSLYVNKHAFHTPAYDVSAFRDLKPLPFFYYSTTPCEVHGNGSMIEDIDYVPLKSAVCITACNLGGAVCRKHATEYREYMEAYNLVSASGFRLWCYKTFDIYNLWSTFTKVQGLENIAFNVVKQGHFIGVEGELPVAVVNDKIFTKSGVNDICMFENKTTLPTNIAFELYAKRAVRSHPDFKLLHNLQADICYKFVLWDYERSNIYGTATIGVCKYTDIDVNSALNICFDIRDNCSLEKFMSTPNAIFISDRKIKKYPCMVGPDYAYFNGAIIRDSDVVKQPVKFYLYKKVNNEFIDPTECIYTQSRSCSDFLPLSDMEKDFLSFDSDVFIKKYGLENYAFEHVVYGDFSHTTLGGLHLLIGLYKKQQEGHIIMEEMLKGSSTIHNYFITETNTAAFKAVCSVIDLKLDDFVMILKSQDLGVVSKVVKVPIDLTMIEFMLWCKDGQVQTFYPRLQASADWKPGHAMPSLFKVQNVNLERCELANYKQSIPMPRGVHMNIAKYMQLCQYLNTCTLAVPANMRVIHFGAGSDKGIAPGTSVLRQWLPTDAIIIDNDLNEFVSDADITLFGDCVTVRVGQQVDLVISDMYDPTTKNVTGSNESKALFFTYLCNLINNNLALGGSVAIKITEHSWSVELYELMGKFAWWTVFCTNANASSSEGFLLGINYLGTIKENIDGGAMHANYIFWRNSTPMNLSTYSLFDLSKFQLKLKGTPVLQLKESQINELVISLLSQGKLLIRDNDTLSVSTDVLVNTYRKLR

SEQ ID NO: 16 - protein, 1353 aa, unidentified
MIHSVFLLMFLLTPTESYVDVGPDSVKSACIEVDIQQPFFDKTWPRPIDVSKADGIIYPQGRTYSYITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQRDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLALLEPVSTSTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFQKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGFLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 17 - protein, 103 aa, unidentified; identical to SEQ ID NO: 6

SEQ ID NO: 18 - protein, 109 aa, unidentified; identical to SEQ ID NO: 7

SEQ ID NO: 19 - protein, 246 aa, unidentified; identical to SEQ ID NO: 8

SEQ ID NO: 20 - protein, 224 aa, unidentified; identical to SEQ ID NO: 9

SEQ ID NO: 21 - protein, 82 aa, unidentified; identical to SEQ ID NO: 10

SEQ ID NO: 22 - protein, 219 aa, unidentified
MSNMMQLTEAQIIAIIKDWNFAWSLIFLLITIVLQYGYPSRSMTVYVFKMFVLWLLWPSSMALSIFSAVYPIDLASQIISGIVAAVSAMMWISYFVQSIRLFMRTGSWWSFNPETNCLLNVPFGGTTVVRPLVEDSTSVTAVVTNGHLKMAGMHFGACDYDRLPNEVTVAKPNVLIALKMVKRQSYGTNSGVAIYHRYKAGNYRSPPITADIELALLRA

SEQ ID NO: 23 - protein, 413 aa, unidentified; identical to SEQ ID NO: 12

SEQ ID NO: 24 - protein, 112 aa, unidentified; identical to SEQ ID NO: 13